(19) United States
(12) Patent Application Publication
(10) Pub. No.: US 2024/0191230 A1
(43) Pub. Date: Jun. 13, 2024

(54) CONJUGATES OF SIRNA AND ANTISENSE OLIGONUCLEOTIDES (SIRNASO) AND METHODS OF USE IN GENE SILENCING

(71) Applicant: UNIVERSITY OF MASSACHUSETTS, Boston, MA (US)

(72) Inventors: Jonathan Watts, Worcester, MA (US); Io Long Chan, Worcester, MA (US); Feng Wang, Worcester, MA (US); Minwook Shin, Boston, MA (US)

(21) Appl. No.: 18/380,871

(22) Filed: Oct. 17, 2023

Related U.S. Application Data
(60) Provisional application No. 63/416,664, filed on Oct. 17, 2022.

(51) Int. Cl. C12N 15/113

(52) U.S. Cl. CPC C12N 15/113; C12N 2310/11; C12N 2310/14; C12N 2310/344; C12N 2310/3519

(57) ABSTRACT

This disclosure relates to compounds (“siRNASO”) comprising a double stranded RNA (dsRNA) attached to antisense oligonucleotides (ASO) and heteroduplex oligonucleotides (HDO). Also provided are methods of inhibiting gene expression with the same.

### CROSS REFERENCE TO RELATED APPLICATION

This application claims the benefit of U.S. Provisional Patent Application Ser. No. 63/416,664, filed Oct. 17, 2022. The entire contents of the above-referenced patent application are incorporated by reference in their entirety herein.

### STATEMENT OF FEDERALLY SPONSORED RESEARCH

This invention was made with government support under Grant No. NS111990 awarded by the National Institutes of Health. The Government has certain rights in the invention.

### BACKGROUND

Oligonucleotide drugs promise to revolutionize personalized medicine through rational design of drugs based on individual genetic sequences. The chemical architecture of synthetic oligonucleotides is under continuous development for improving safety and potency. A general usage of oligonucleotide therapies is to modulate gene expression and correct unfavorable transcriptomic environments that cause diseased states. Two popular classes of oligonucleotide drugs, i.e., small interfering RNAs (siRNA) and antisense oligonucleotides (ASO), are commonly used for targeted gene silencing and act through distinct mechanisms, and expansive applications of ASOs beyond silencing include modulation of RNA splicing, microRNA inhibition, enhancing translation, RNA editing, and much more.

To date, most oligonucleotide technologies focus on single gene targets. Attempts to enhance single gene silencing or to enable targeting of multiple transcripts using multimeric oligonucleotides conjugate the same class of oligonucleotide structures—i.e., linking siRNA to siRNA or ASO to ASO. However, novel methods of targeting single genes with multiple oligonucleotide structures, or targeting two or more genes with multiple oligonucleotide structures, are needed in the field.

### SUMMARY

Provided herein is a novel conjugated form of siRNA and ASO coined “siRNASO” to achieve unprecedented silencing efficacy in vivo against single gene targets and expanded utility of siRNASO for modulating multiple genes through distinct mechanisms by altering structures of ASOs.

The single gene-targeting siRNASO compounds can achieve greater therapeutic effect via greater gene silencing efficacy. This is driven by greater bioavailability as well as the ability to target both cytoplasmic and nuclear pools of RNA targets through the unique mechanisms of siRNAs and ASOs, thus expanding the potential of therapeutic interventions through combinatorial RNA targeting as siRNA and ASO technologies continue to mature and continuous better understanding of disease genetics.

Furthermore, single gene targeting siRNA and ASO drug candidates fail to address complex diseases. The siRNASO platform provides a controllable method for co-delivering potent gene modulating oligonucleotide agents that allow precise manipulation of gene expression of single or multiple gene targets within recipient cells.

siRNASOs have numerous advantages over pri-miRNAs that may be processed to release an siRNA and ASO. For instance, the preparation of siRNASOs is based on solid-phase synthesis, which does not rely on enzymes. In contrast, the preparation of pri-miRNAs relies on the enzyme RNA ligase 2 (NEB) to ligate 2 fragments, which is more expensive and less efficient. Furthermore, in cells, pri-miRNAs rely on Dicer and Drosha for cleavage before releasing the functional siRNA and ASO compounds. In contrast, siRNASOs do not require such an extra step, thus making the gene targeting process more efficient. Moreover, siRNASOs show better stability and cell delivery and better tolerate chemical modification when compare with pri-miRNAs.

In one aspect, the disclosure provides a compound comprising: a) an siRNA comprising a sense strand and an antisense strand, and b) an antisense oligonucleotide (ASO), wherein the siRNA is attached to the ASO with a linker.

In certain embodiments, the linker is a non-nucleotide linker. In certain embodiments, the non-nucleotide linker comprises an ethylene glycol chain, an alkyl chain, a peptide, an amide, a carbamate, or a combination thereof. In certain embodiments, the alkyl chain is a C2 to C15 alkyl chain. In certain embodiments, the alkyl chain is a propyl (C3), nonyl (C9), or dodecyl (C12) chain. In certain embodiments, the ethylene glycol chain comprises 1-15 ethylene glycol units. In certain embodiments, the ethylene glycol chain is 1 (PEG1), 2 (PEG2), 4 (PEG4), or 12 (PEG12) ethylene glycol units.

In certain embodiments, the linker is a cleavable linker, optionally wherein the cleavable linker comprises a phosphodiester linkage, a disulfide linkage, an acid-labile linkage, a photocleavable linkage, or a (dT)ₙ such as a dTdT dinucleotide with phosphodiester intemucleotide linkages, optionally wherein the acid-labile linkage comprises a β-thiopropionate linkage or a carboxydimethylmaleic anhydride (CDM) linkage.

In certain embodiments, the linker comprises a nucleotide linker. In certain embodiments, the nucleotide linker comprises (dT)n, (dA)n, (dC)n, (dG)n, or (U)n, wherein n is an integer from 2 to 30. In certain embodiments, the nucleotide linker is selected from the group consisting of (dT)₂, (dT)₁₀, (dT)₃₀, (dA)₂, (dA)₁₀, (dC)₂, (dG)₂, (U)₂, (U)₁₀, and (dT)(dA).

In certain embodiments, the nucleotide linker of the compound further comprises a non-nucleotide linker. In certain embodiments, the non-nucleotide linker comprises an ethylene glycol chain, an alkyl chain, a peptide, an amide, a carbamate, or a combination thereof. In certain embodiments, the alkyl chain is a C2 to C15 alkyl chain. In certain embodiments, the alkyl chain is a propyl (C3), nonyl (C9), or dodecyl (C12) chain. In certain embodiments, the ethylene glycol chain comprises 1-15 ethylene glycol units. In certain embodiments, the ethylene glycol chain is 1 (PEG1), 2 (PEG2), 4 (PEG4), or 12 (PEG12) ethylene glycol units.

In certain embodiments, the compound does not comprise a primary microRNA (pri-miRNA) or a pre-microRNA. In certain embodiments, the compound does not comprise a nucleotide hairpin structure.

In certain embodiments, the ASO is linked to the sense strand 5′ or 3′ end.

In certain embodiments, the ASO is linked to the 5′ or 3′ end of the siRNA antisense strand.

In certain embodiments, the antisense strand of the siRNA comprises about 15 nucleotides to 25 nucleotides in length. In certain embodiments, the sense strand comprises about 15 nucleotides to about 25 nucleotides in length. In certain embodiments, the antisense strand of the siRNA is 20 nucleotides in length. In certain embodiments, the antisense strand of the siRNA is 21 nucleotides in length. In certain embodiments, the antisense strand of the siRNA is 22 nucleotides in length. In certain embodiments, the sense strand is 15 nucleotides in length. In certain embodiments, the sense strand is 16 nucleotides in length. In certain embodiments, the sense strand is 18 nucleotides in length. In certain embodiments, the sense strand is 20 nucleotides in length or 21 nucleotides in length.

In certain embodiments, the siRNA comprises a double-stranded region of 15 base pairs to 20 base pairs. In certain embodiments, the siRNA comprises a double-stranded region of 15 base pairs. In certain embodiments, the siRNA comprises a double-stranded region of 16 base pairs. In certain embodiments, the siRNA comprises a double-stranded region of 18 base pairs. In certain embodiments, the siRNA comprises a double-stranded region of 20 base pairs or 21 base pairs.

In certain embodiments, the siRNA comprises a blunt-end.

In certain embodiments, the siRNA comprises at least one single stranded nucleotide overhang. In certain embodiments, the siRNA comprises about a 2-nucleotide to 5-nucleotide single stranded nucleotide overhang. In certain embodiments, the siRNA comprises a 2-nucleotide single stranded nucleotide overhang. In certain embodiments, the siRNA comprises a 5-nucleotide single stranded nucleotide overhang.

In certain embodiments, the siRNA comprises naturally occurring nucleotides.

In certain embodiments, the siRNA comprises at least one modified nucleotide. In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified internucleotide linkage. In certain embodiments, the modified internucleotide linkage comprises a phosphorothioate internucleotide linkage. In certain embodiments, the compound comprises about 4-16 phosphorothioate internucleotide linkages. In certain embodiments, the siRNA comprises about 4-16 phosphorothioate internucleotide linkages. In certain embodiments, the compound comprises about 4-13 phosphorothioate internucleotide linkages, optionally wherein the siRNA comprises 8 or 13 phosphorothioate internucleotide linkages.

In certain embodiments, the siRNA comprises at least 80% chemically modified nucleotides. In certain embodiments, the siRNA is fully chemically modified. In certain embodiments, the siRNA comprises at least 70% 2′-O-methyl nucleotide modifications. In certain embodiments, the antisense strand of the siRNA comprises at least 70% 2′-O-methyl nucleotide modifications. In certain embodiments, the antisense strand of the siRNA comprises about 70% to 90% 2′-O-methyl nucleotide modifications. In certain embodiments, the sense strand comprises at least 65% 2′-O-methyl nucleotide modifications. In certain embodiments, the sense strand comprises 100% 2′-O-methyl nucleotide modifications.

In certain embodiments, the sense strand comprises one or more nucleotide mismatches between the antisense strand and the sense strand of the siRNA.

In certain embodiments, the antisense strand of the siRNA comprises a 5′ phosphate, a 5′-phosphoramidate, a 5′-alkyl phosphonate, a 5′ alkylene phosphonate, or a 5′ alkenyl phosphonate. In certain embodiments, the antisense strand of the siRNA comprises a 5′ vinyl phosphonate.

In certain embodiments, a functional moiety is linked to the 5′ end and/or 3′ end of the siRNA antisense strand. In certain embodiments, a functional moiety is linked to the 5′ end and/or 3′ end of the siRNA sense strand. In certain embodiments, a functional moiety is linked to the 3′ end of the siRNA sense strand.

In certain embodiments, the functional moiety comprises an N-acetylgalactosamine (GalNAc) moiety.

In certain embodiments, the functional moiety comprises a hydrophobic moiety.

In certain embodiments, the hydrophobic moiety is selected from the group consisting of fatty acids, steroids, secosteroids, lipids, gangliosides, nucleoside analogs, endocannabinoids, vitamins, peptides, antibodies, antibody fragments, carbohydrate ligands, and a mixture thereof.

In certain embodiments, the steroid is selected from the group consisting of cholesterol and Lithocholic acid (LCA).

In certain embodiments, the fatty acid is selected from the group consisting of Eicosapentaenoic acid (EPA), Docosahexaenoic acid (DHA), and Docosanoic acid (DCA).

In certain embodiments, the functional moiety is linked to the antisense strand and/or sense strand of the siRNA by a linker.

In certain embodiments, the linker is a cleavable linker, optionally wherein the cleavable linker comprises a phosphodiester linkage, a disulfide linkage, an acid-labile linkage, a photocleavable linkage, or (dT)₁ such as a dTdT dinucleotide with phosphodiester intemucleotide linkages, optionally wherein the acid-labile linkage comprises a β-thiopropionate linkage or a carboxydimethylmaleic anhydride (CDM) linkage.

In certain embodiments, the linker comprises a divalent or trivalent linker, optionally wherein the divalent or trivalent linker is selected from the group consisting of

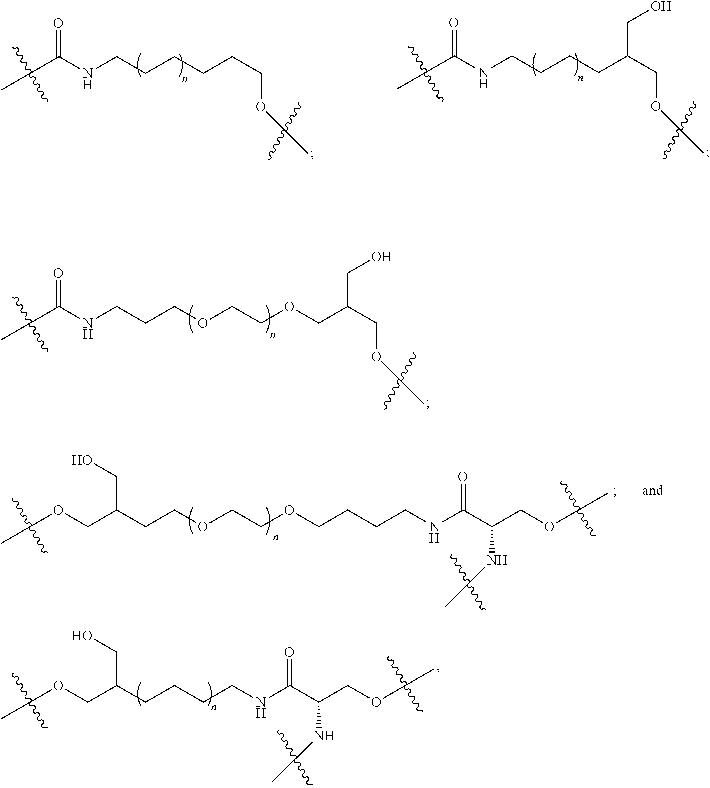

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, the linker further links a phosphodiester or phosphodiester derivative if the linker is a trivalent linker.

In certain embodiments, the phosphodiester or phosphodiester derivative is selected from the group consisting of

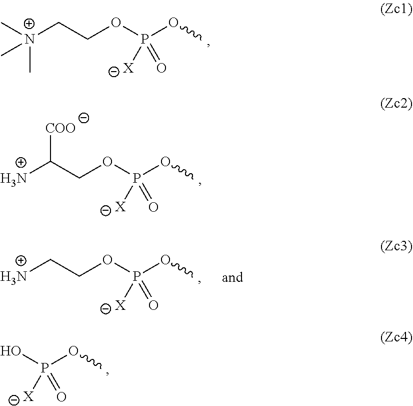

wherein X is O, S, or BH₃.

In certain embodiments, the nucleotides at positions 1 and 2 from the 3′ end of sense strand, and the nucleotides at positions 1 and 2 from the 5′ end of the antisense strand of the siRNA are connected to adjacent ribonucleotides via phosphorothioate linkages.

In certain embodiments, the antisense oligonucleotide is between about 8 nucleotides to 80 nucleotides in length.

In certain embodiments, the antisense oligonucleotide is between about 10 nucleotides to 30 nucleotides in length.

In certain embodiments, the antisense oligonucleotide comprises one or more modified nucleotides.

In certain embodiments, the one or more modified nucleotides each independently comprise a modification of a ribose group, a phosphate group, a nucleobase, or a combination thereof.

In certain embodiments, each modification of the ribose group comprises a 2′-O-methyl, a 2′-fluoro, 2′-deoxy, a 2′-O-(2-methoxyethyl) (MOE), a 2′-O-alkyl, a 2′-O-alkoxy, a 2′-O-alkylamino, a 2′-NH₂, a constrained nucleotide, or a combination thereof.

In certain embodiments, the constrained nucleotide comprises a locked nucleic acid (LNA), an ethyl-constrained nucleotide, a 2′-(S)-constrained ethyl (S-cEt) nucleotide, a constrained MOE, a 2′-0,4′-C-aminomethylene bridged nucleic acid (2′,4′-BNA^{NC}), an alpha-L-locked nucleic acid, a tricyclo-DNA, or a combination thereof.

In certain embodiments, the modification of the ribose group comprises a 2′-O-(2-methoxyethyl) (MOE) modification.

In certain embodiments, the modification of the ribose group comprises a 2′-deoxy modification.

In certain embodiments, each modification of the phosphate group comprises a phosphorothioate, a phosphonoacetate (PACE), a thiophosphonoacetate (thioPACE), an amide, a triazole, a phosphonate, a phosphotriester, or a combination thereof.

In certain embodiments, the modification of the phosphate group comprises a phosphorothioate.

In certain embodiments, every nucleotide of the antisense oligonucleotide comprises a phosphorothioate.

In certain embodiments, the antisense oligonucleotide comprises a combination of phosphorothioate and non-phosphorothioate linkages.

In certain embodiments, the antisense oligonucleotide comprises a combination of phosphorothioate and phosphoramidate linkages.

In certain embodiments, the antisense oligonucleotide comprises a combination of phosphorothioate and phosphodiester linkages.

In certain embodiments, each modification of the nucleobase comprises a 2-thiouridine, a 4-thiouridine, a N⁶-methyladenosine, a pseudouridine, a 2,6-diaminopurine, inosine, a thymidine, a 5-methylcytosine, a 5-substituted pyrimidine, an isoguanine, an isocytosine, a halogenated aromatic group, or a combination thereof.

In certain embodiments, the modification of the nucleobase group comprises a 5-methylcytosine modification.

In certain embodiments, the antisense oligonucleotide comprises the formula: A-B-C, wherein: A comprises from about 0 to about 8 modified nucleotides; B comprises from about 6 to about 18 deoxyribonucleic acid (DNA) nucleotides and/or DNA-like nucleotides; and C comprises from about 0 to about 8 modified nucleotides; and wherein the overall length of the antisense oligonucleotide is about 12 to about 30 nucleotides.

In certain embodiments, A comprises from about 2 to about 6 modified nucleotides, B comprises from about 6 to about 12 DNA nucleotides and/or DNA-like nucleotides, and C comprises from about 2 to about 6 modified nucleotides.

In certain embodiments, A comprises or consists of 3 modified nucleotides, B comprises or consists of 10 DNA nucleotides and/or DNA-like nucleotides, and C comprises or consists of 3 modified nucleotides.

In certain embodiments, A comprises or consists of 5 modified nucleotides, B comprises or consists of 10 DNA nucleotides and/or DNA-like nucleotides, and C comprises or consists of 5 modified nucleotides.

In certain embodiments, A comprises from about 2 to about 6 2′-O-(2-methoxyethyl) (MOE) modified nucleotides, B comprises from about 6 to about 12 DNA nucleotides and/or DNA-like nucleotides, and C comprises from about 2 to about 6 2′-O-(2-methoxyethyl) (MOE) modified nucleotides.

In certain embodiments, A comprises or consists of 3 2′-O-(2-methoxyethyl) (MOE) modified nucleotides, B comprises or consists of 10 DNA nucleotides and/or DNA-like nucleotides, and C comprises or consists of 3 2′-O-(2-methoxyethyl) (MOE) modified nucleotides.

In certain embodiments, A comprises or consists of 5 2′-O-(2-methoxyethyl) (MOE) modified nucleotides, B comprises or consists of 10 DNA nucleotides and/or DNA-like nucleotides, and C comprises or consists of 5 2′-O-(2-methoxyethyl) (MOE) modified nucleotides.

In certain embodiments, A comprises from about 2 to about 6 LNA modified nucleotides, B comprises from about 6 to about 12 DNA nucleotides and/or DNA-like nucleotides, and C comprises from about 2 to about 6 LNA modified nucleotides.

In certain embodiments, rein A comprises or consists of 3 LNA modified nucleotides, B comprises or consists of 10 DNA nucleotides and/or DNA-like nucleotides, and C comprises or consists of 3 LNA modified nucleotides.

In certain embodiments, the antisense oligonucleotide is a splice-modulating oligonucleotide.

In certain embodiments, the antisense oligonucleotide is a steric blocking oligonucleotide that elicits gene activation.

In certain embodiments, the antisense oligonucleotide is designed to recruit RNase H to reduce the expression of a complementary transcript.

In certain embodiments, the antisense oligonucleotide comprises the formula: A-B-C, wherein: A comprises from about 0 to about 8 modified nucleotides; B comprises from about 6 to about 18 deoxyribonucleic acid (DNA) nucleotides and/or DNA-like nucleotides; and C comprises from about 0 to about 8 modified nucleotides; and wherein the overall length of the antisense oligonucleotide is about 12 to about 30 nucleotides.

In certain embodiments, the compound further comprises a complementary oligonucleotide annealed to the antisense oligonucleotide.

In certain embodiments, the complementary oligonucleotide is linked to the sense strand 5′ or 3′ end.

In certain embodiments, the complementary oligonucleotide is linked to the 5′ or 3′ end of the siRNA antisense strand.

In certain embodiments, the compound further comprises at least one additional siRNA linked to the compound.

In certain embodiments, the compound further comprises at least one additional ASO linked to the compound.

In certain embodiments, the compound comprises or consisting of a first ASO, a first linker, a second ASO, a second linker, and a first siRNA comprising a first antisense strand and a first sense strand, wherein the 5′ end of the first ASO is linked to the 3′ end of the second ASO via the first linker and the 5′ end of the second ASO is linked to the 3′ end of the first sense strand via the second linker.

In certain embodiments, the compound comprises or consisting of a first siRNA comprising a first antisense strand and a first sense strand, a first linker, a first ASO, a second linker, and a second siRNA comprising a second antisense strand and a second sense strand, wherein the 5′ end of the first sense strand is linked to the 3′ end of the first ASO via the first linker and the 5′ end of the first ASO is linked to the 3′ end of the second sense strand via the second linker.

In certain embodiments, the compound comprises or consisting of a first ASO, a first linker, a first siRNA comprising a first antisense strand and a first sense strand, a second linker, a second ASO, a third linker, and a second siRNA comprising a second antisense strand and a second sense strand, wherein the 5′ end of the first ASO is linked to the 3′ end of the first sense strand via the first linker, the 5′ end of the first sense strand is linked to the 3′ end of the second ASO via the second linker, and the 5′ end of the second ASO is linked to the 3′ end of the second sense strand via the third linker.

In certain embodiments, the compound comprises or consisting of a first siRNA comprising a first antisense strand and a first sense strand, a first linker, a first ASO, a second linker, a second ASO, a third linker, and a second siRNA comprising a second antisense strand and a second sense strand, wherein the 3′ end of the first sense strand is linked to the 5′ end of the first ASO via the first linker, the 3′ end of the first ASO is linked to the 3′ end of the second ASO via the second linker, and the 5′ end of the second ASO is linked to the 3′ end of the second sense strand via the third linker.

In certain embodiments, the first siRNA and the second siRNA have identical nucleotide sequences.

In certain embodiments, the first siRNA and the second siRNA have complementarity to the same target gene.

In certain embodiments, the first siRNA and the second siRNA have different nucleotide sequences.

In certain embodiments, the first siRNA and the second siRNA have complementarity to the different target genes.

In certain embodiments, the first ASO and the second ASO have identical nucleotide sequences.

In certain embodiments, the first ASO and the second ASO have complementarity to the same target gene.

In certain embodiments, the first ASO and the second ASO have different nucleotide sequences.

In certain embodiments, the first ASO and the second ASO have complementarity to the different target genes.

In certain embodiments, the first linker, the second linker, and the third linker are identical.

In certain embodiments, the first linker, the second linker, and the third linker are different.

In one aspect, the disclosure provides a compound comprising: a) an siRNA comprising a sense strand and an antisense strand, and b) a heteroduplex oligonucleotide (HDO) comprising an antisense oligonucleotide (ASO) and a complementary oligonucleotide, wherein the siRNA is attached to the HDO with a linker.

In certain embodiments, the linker is anon-nucleotide linker. In certain embodiments, the non-nucleotide linker comprises an ethylene glycol chain, an alkyl chain, a peptide, an amide, a carbamate, or a combination thereof.

In certain embodiments, the linker is a cleavable linker, optionally wherein the cleavable linker comprises a phosphodiester linkage, a disulfide linkage, an acid-labile linkage, a photocleavable linkage, or (dT)₁ such as a dTdT dinucleotide with phosphodiester intemucleotide linkages, optionally wherein the acid-labile linkage comprises a β-thiopropionate linkage or a carboxydimethylmaleic anhydride (CDM) linkage.

In certain embodiments, the compound does not comprise a primary microRNA (pri-miRNA) or a pre-microRNA.

In certain embodiments, the complementary oligonucleotide is linked to the sense strand 5′ or 3′ end.

In certain embodiments, the complementary oligonucleotide is linked to the 5′ or 3′ end of the siRNA antisense strand.

In certain embodiments, the HDO comprises one or more modified nucleotides.

In certain embodiments, the one or more modified nucleotides each independently comprise a modification of a ribose group, a phosphate group, a nucleobase, or a combination thereof.

In certain embodiments, each modification of the ribose group comprises 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH₂, a constrained nucleotide, or a combination thereof.

In certain embodiments, the constrained nucleotide comprises a locked nucleic acid (LNA), an ethyl-constrained nucleotide, a 2′-(S)-constrained ethyl (S-cEt) nucleotide, a constrained MOE, a 2′-0,4′-C-aminomethylene bridged nucleic acid (2′,4′-BNA^{NC}), an alpha-L-locked nucleic acid, a tricyclo-DNA, or a combination thereof.

In certain embodiments, the modification of the ribose group comprises a 2′-O-(2-methoxyethyl) (MOE) modification.

In certain embodiments, the modification of the ribose group comprises a 2′-deoxy modification.

In certain embodiments, each modification of the phosphate group comprises a phosphorothioate, a phosphonoacetate (PACE), a thiophosphonoacetate (thioPACE), an amide, a triazole, a phosphonate, a phosphotriester, or a combination thereof.

In certain embodiments, the modification of the phosphate group comprises a phosphorothioate.

In certain embodiments, every nucleotide of the antisense oligonucleotide comprises a phosphorothioate.

In certain embodiments, each modification of the nucleobase comprises a 2-thiouridine, a 4-thiouridine, a N⁶-methyladenosine, a pseudouridine, a 2,6-diaminopurine, inosine, a thymidine, a 5-methylcytosine, a 5-substituted pyrimidine, an isoguanine, an isocytosine, a halogenated aromatic group, or a combination thereof.

In certain embodiments, the modification of the nucleobase group comprises a 5-methylcytosine modification.

In certain embodiments, the HDO comprises at least 80% chemically modified nucleotides. In certain embodiments, the HDO is fully chemically modified.

In certain embodiments, the HDO comprises at least 70% 2′-O-methyl nucleotide modifications.

In certain embodiments, the HDO comprises at least one modified internucleotide linkage.

In certain embodiments, the modified internucleotide linkage comprises a phosphorothioate internucleotide linkage.

In certain embodiments, the compound further comprises a second antisense oligonucleotide (ASO), wherein the second ASO is attached to the HDO and/or siRNA with a linker.

In one aspect, the disclosure provides a method for inhibiting expression of a target gene in a cell, the method comprising: (a) introducing into the cell the compound described above; and (b) maintaining the cell produced in step (a) for a time sufficient to obtain degradation of the mRNA transcript of the target gene, thereby inhibiting expression of the target gene in the cell.

In one aspect, the disclosure provides a method for inhibiting expression of a target gene in a subject, the method comprising administering to the subject the compound described above, thereby inhibiting expression of the target gene in the subject.

In one aspect, the disclosure provides a method for inhibiting expression of a target gene in a tissue of a subject, the method comprising administering to the subject the compound described above, thereby inhibiting expression of the target gene in the tissue.

In certain embodiments, the tissue is selected from the group consisting of lung tissue, liver tissue, placenta tissue, kidney tissue, spleen tissue, and brain tissue.

In certain embodiments, the compound is administered intravenously or subcutaneously.

In certain embodiments, the tissue is lung tissue and the compound is administered intratracheally.

In certain embodiments, the compound inhibits expression of the target gene greater than: i) an siRNA targeting the target gene alone; ii) an ASO targeting the target gene alone; or iii) a combination of the siRNA and the ASO.

### BRIEF DESCRIPTION OF THE DRAWINGS

The foregoing and other features and advantages of the present disclosure will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIGS. 1A-B depict schematics of siRNASO compounds, according to one or more embodiments of the present disclosure. FIG. 1A shows the structure of ID2 1-4 and

FIG. 1B shows the structure of Di-siRNASO and Groups 1-5. Conjugations can be added at any 5′ and 3′ end, linker, or any nucleotide/non-nucleotide site of all claimed structures.

FIG. 2 depicts relative RNA expression of Htt (left bar for each data point) and Malat1 (right bar for each data point), each normalized Hprt expression. UTC represents untreated control. NTC1 represents a non-treatment control with siRNA and ASO that do not target Htt or Malat1.

FIG. 3 depicts a schematic of five different heteroduplex oligonucleotide (HDO)-based siRNASO compounds.

FIG. 4 depicts relative RNA expression of Htt (left bar for each data point) and Malat1 (right bar for each data point), each normalized Hprt expression. UTC represents untreated control. NTC1 represents a non-treatment control with siRNA and HDO that do not target Htt or Malat1.

FIGS. 5A-D depicts relative RNA expression of Adam33 in mouse lung tissue after intratracheal administration of siRNASOs with different structures at 2.5 nmol (FIG. 5A-B, Adam33 expression analyzed at 10 days post administration (n=6); In FIG. 5A, Adam33 expression was analyzed using RT-qPCR normalized to the expression of Ppib; In FIG. 5B, Adam33 expression was analyzed using ddPCR normalized to the expression of Gusb) and of siRNASO1 at different doses (FIG. 5C, Adam33 expression analyzed at 7 days post administration (n=4)) and duration of effect assessed for up to 12 weeks (FIG. 5D).

FIGS. 6A-B depicts relative RNA expression of Cd47 in mouse lung tissue after intratracheal administration of Cd47-targeting siRNASOs with four different structures at three doses (FIG. 6A, Cd47 expression analyzed at 7 days post administration (n=3); FIG. 6B depicts relative RNA expression of Cd47 in mouse lung tissue after intratracheal administration of Cd47-targeting siRNASO at 5 nmol of active arms (FIG. 6B, Cd47 expression analyzed at 7 days post administration (n=6)).

FIG. 7 depicts relative RNA expression of Loxl2 in mouse lung tissue after intratracheal administration of Loxl2-targeting siRNASO at 20 nmol of active arms (FIG. 7, Loxl2 expression analyzed at 10 days post administration (n=6)).

FIGS. 8A-B depict relative RNA expression of Atxn2 in the mouse brain following Bi-ICV injection followed by two-week incubation. FIG. 8A compares di-siRNA targeting Atxn2 to an siRNASO with the same siRNA targeting Atxn2. FIG. 8B compares an ASO targeting Atxn2 to an siRNASO with an siRNA targeting Atxn2.

FIGS. 9A-B depicts extensive analysis of linker structures and top candidates that perform significantly better than the parent structure PEG4-dTdT. FIG. 9A depicts relative RNA expression of Adam33 in mouse lung tissue after intratracheal administration of Adam33-targeting siRNASO at 2.5 nmol (Adam33 expression analyzed at 30 days post administration (n=5)). All groups showed significant silencing compared to PBS control group (p<0.0001). Linker structures tested contained various lengths of polyethylene glycol (PEG) and alkyl (C3=propyl, C9=nonyl, C12=dodecyl). All linkers contained dTdT di-nucleotide except for PEG4 only. FIG. 9B depicts post hoc analysis of the top two linkers relative to first generation linker PEG4-dTdT.

FIGS. 10A-B depict relative RNA expression of Atxn2 RNA in the mouse brain following Bi-ICV injection followed by two-week incubation. FIG. 10A depicts the structures of siRNASO tested. FIG. 10B depicts silencing of the Atxn2 RNA in various structures of the mouse brain.

FIGS. 11A-C depict I1-6 expression and Cd47 and Cd98 dual-gene targeting in mouse lung via bulk lung analysis.

FIGS. 12A-H depict Cd47 and Cd98 dual-gene targeting in mouse lung via cell-type specific analysis.

FIGS. 13A-B depict relative RNA expression of Malat1 (FIG. 13A) and Atxn2 (FIG. 13B) RNA in the mouse brain following Bi-ICV injection at 20 nmol followed by two-week incubation. Incubation was with an siRNASO with an siRNA targeting Atxn2 and an ASO targeting Malat1.

FIGS. 14A-B depict silencing & exon skipping by siRNASO (siA39, 33 nmol of intact molecule) in mouse brain. SiRNA arm targeted Akt1 and ASO arm induced exon skipping of Cln3. FIG. 14A depicts the RNA expression of Akt1 that is targeted by the siRNA arm. FIG. 14B depicts PCR result of Cln3 that is targeted by the ASO arm from cDNA samples from the motor cortex of indicated mice. Data was measured in the mouse brain following Bi-ICV injection at 33 nmol followed by two-week incubation.

FIGS. 15 A-F depicts the silencing & RNA editing by siRNASO transfected in ARPE-19 cells. FIGS. 15 A-B, silencing and RNA editing by siRNASO (siA41). ASO arm targets ACTB and siRNA arm targets AKT1. FIGS. 15 C-D, silencing and RNA editing by siRNASO (siA45). ASO arm targets ACTB and siRNA arm targets ATXN2. FIGS. 15 E-F, silencing and RNA editing by siRNASO (siA47). ASO arm targets ACTB and siRNA arm targets HTT.

FIGS. 16A-E depicts the short- and long-term safety profile of siRNASO in mouse CNS. FIG. 16A, with doses ranging from 200-675 μg and various preparation methods, siRNASO shows 100% survival in all conditions. FIG. 16B-C, siRNASO shows safe recovery similar to the aCSF1 group. FIG. 16D-E, siRNASO shows no elevated neuroinflammation makers (Gfap, Iba1) at 2 weeks post ICV injection.

FIG. 17 depicts siRNA accumulation from the siRNASO in the mouse lung after 1 and 2 weeks. Compared against mixed but non-conjugated siRNA comparator group (mono asymmetric siRNA). Administered intratracheal, 15 or 7.5 nmol, n=3, 1 week, PNA hybridization assay, 21/16 antisense strand/sense strand length with cleavable linker.

### DETAILED DESCRIPTION

Unless otherwise specified, nomenclature used in connection with cell and tissue culture, molecular biology, immunology, microbiology, genetics, and protein and nucleic acid chemistry and hybridization described herein are those well-known and commonly used in the art. Unless otherwise specified, the methods and techniques provided herein are performed according to conventional methods well-known in the art and as described in various general and more specific references that are cited and discussed throughout the present specification. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

### I. Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide that may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified nucleotides (e.g., alkylated, e.g., N6-methyl adenosine, or as otherwise known in the art); and other heterocyclically modified nucleotide analogs, such as those described in Herdewijn, Antisense Nucleic Acid Drug Dev., 2000 Aug. 10(4):297-310.

Nucleotide analogs may also comprise modifications to the sugar portion of the nucleotide. For example, the 2′ OH-group may be replaced by a group selected from H, OR, R, F, Cl, Br, I, SH, SR, NH₂, NHR, NR₂, or COOR, wherein R is substituted or unsubstituted C1-C6 alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions, which allow the nucleotide to perform its intended function, such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “oligonucleotide” refers to a short polymer of nucleotides and/or nucleotide analogs.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” “(“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An siRNA, has an antisense strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target” polynucleotide sequence,” e.g., when both polynucleotide sequences are present in the same cell. In certain embodiments, the target polynucleotide sequence corresponds to a target gene, while the non-target polynucleotide sequence corresponds to a non-target gene. In other embodiments, the target polynucleotide sequence corresponds to a target allele, while the non-target polynucleotide sequence corresponds to a non-target allele. In certain embodiments, the target polynucleotide sequence is the DNA sequence encoding the regulatory region (e.g. promoter or enhancer elements) of a target gene. In other embodiments, the target polynucleotide sequence is a target mRNA encoded by a target gene.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “transgene” refers to any nucleic acid molecule, which is inserted by artifice into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term “transgene” also means a nucleic acid molecule that includes one or more selected nucleic acid sequences, e.g., DNAs, that encode one or more engineered RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected nucleic acid sequence, all operably linked to the selected sequence, and may include an enhancer sequence.

A gene “involved” in a disease or disorder includes a gene, the normal or aberrant expression or function of which effects or causes the disease or disorder or at least one symptom of said disease or disorder.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g. an orthologue or paralogue) of the target gene.

The phrase “examining the function of a gene in a cell or organism” refers to examining or studying the expression, activity, function or phenotype arising therefrom.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, antisense oligonucleotides, gapmer molecules, and dual-function oligonucleotides, as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and 2,2N,N-dimethylguanosine.

As used herein, the term “antisense strand” of an siRNA, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an siRNA, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA strand having sufficient complementarity to form a duplex with the miRNA strand.

As used herein, the term “guide strand” refers to a strand of an siRNA, e.g., an antisense strand of an siRNA duplex or siRNA sequence, that enters into the RISC complex and directs cleavage of the target mRNA.

As used herein, the term “asymmetry,” as in the asymmetry of the duplex region of an siRNA, refers to an inequality of bond strength or base pairing strength between the termini of the siRNA (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like, between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog, such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g. certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moiety.

As used herein, the terms “sufficient complementarity” or “sufficient degree of complementarity” mean that the RNA silencing agent has a sequence (e.g. in the antisense strand of an siRNA or the antisense oligonucleotide), which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from short hairpin RNA (shRNA) precursors. Both RNAi and translational repression are mediated by RNA-induced silencing complex (RISC). Both RNAi and translational repression occur naturally or can be initiated by the hand of man, for example, to silence the expression of target genes.

Various methodologies of the instant disclosure include a step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and example are illustrative only and not intended to be limiting.

Various aspects of the disclosure are described in further detail in the following sections.

### II. siRNASO—siRNA Components

### siRNA Design

In some embodiments, siRNAs are designed as follows. First, a portion of the target gene is selected. Cleavage of mRNA at these sites should eliminate translation of corresponding protein. Antisense strands were designed based on the target sequence and sense strands were designed to be complementary to the antisense strand. Hybridization of the antisense and sense strands forms the siRNA duplex. The antisense strand includes about 19 to 25 nucleotides, e.g., 19, 20, 21, 22, 23, 24 or 25 nucleotides. In other embodiments, the antisense strand includes 20, 21, 22 or 23 nucleotides. The sense strand includes about 14 to 25 nucleotides, e.g., 14, 15, 16, 17, 18, 19, 20, 21, 22, 23, 24 or 25 nucleotides. In other embodiments, the sense strand is 15 nucleotides. In other embodiments, the sense strand is 18 nucleotides. In other embodiments, the sense strand is 20 nucleotides. The skilled artisan will appreciate, however, that siRNAs having a length of less than 19 nucleotides or greater than 25 nucleotides can also function to mediate RNAi. Accordingly, siRNAs of such length are also within the scope of the instant disclosure, provided that they retain the ability to mediate RNAi. Longer siRNA have been demonstrated to elicit an interferon or PKR response in certain mammalian cells, which may be undesirable. In certain embodiments, the siRNA of this disclosure do not elicit a PKR response (i.e., are of a sufficiently short length). However, longer siRNA may be useful, for example, in cell types incapable of generating a PKR response or in situations where the PKR response has been down-regulated or dampened by alternative means.

The sense strand sequence can be designed such that the target sequence is essentially in the middle of the strand. Moving the target sequence to an off-center position can, in some instances, reduce efficiency of cleavage by the siRNA. Such compositions, i.e., less efficient compositions, may be desirable for use if off-silencing of the wild-type mRNA is detected.

The antisense strand can be the same length as the sense strand and includes complementary nucleotides. In one embodiment, the strands are fully complementary, i.e., the strands are blunt-ended when aligned or annealed. In another embodiment, the strands align or anneal such that 1-, 2-, 3-, 4-, 5-, 6-, 7-, or 8-nucleotide overhangs are generated, i.e., the 3′ end of the sense strand extends 1, 2, 3, 4, 5, 6, 7, or 8 nucleotides further than the 5′ end of the antisense strand and/or the 3′ end of the antisense strand extends 1, 2, 3, 4, 5, 6, 7, or 8 nucleotides further than the 5′ end of the sense strand. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material.

To facilitate entry of the antisense strand into RISC (and thus increase or improve the efficiency of target cleavage and silencing), the base pair strength between the 5′ end of the sense strand and 3′ end of the antisense strand can be altered, e.g., lessened or reduced, as described in detail in U.S. Pat. Nos. 7,459,547, 7,772,203 and 7,732,593, entitled “Methods and Compositions for Controlling Efficacy of RNA Silencing” (filed Jun. 2, 2003) and U.S. Pat. Nos. 8,309,704, 7,750,144, 8,304,530, 8,329,892 and 8,309,705, entitled “Methods and Compositions for Enhancing the Efficacy and Specificity of RNAi” (filed Jun. 2, 2003), the contents of which are incorporated in their entirety by this reference. In one embodiment of these aspects of the disclosure, the base-pair strength is less due to fewer G:C base pairs between the 5′ end of the first or antisense strand and the 3′ end of the second or sense strand than between the 3′ end of the first or antisense strand and the 5′ end of the second or sense strand. In another embodiment, the base pair strength is less due to at least one mismatched base pair between the 5′ end of the first or antisense strand and the 3′ end of the second or sense strand. In certain exemplary embodiments, the mismatched base pair is selected from the group consisting of G:A, C:A, C:U, G:G, A:A, C:C and U:U. In another embodiment, the base pair strength is less due to at least one wobble base pair, e.g., G:U, between the 5′ end of the first or antisense strand and the 3′ end of the second or sense strand. In another embodiment, the base pair strength is less due to at least one base pair comprising a rare nucleotide, e.g., inosine (I). In certain exemplary embodiments, the base pair is selected from the group consisting of an I:A, I:U and I:C. In yet another embodiment, the base pair strength is less due to at least one base pair comprising a modified nucleotide. In certain exemplary embodiments, the modified nucleotide is selected from the group consisting of 2-amino-G, 2-amino-A, 2,6-diamino-G, and 2,6-diamino-A.

To validate the effectiveness by which siRNAs destroy mRNAs, the siRNA can be incubated with cDNA in a Drosophila-based in vitro mRNA expression system. Radiolabeled with ³²P, newly synthesized mRNAs are detected autoradiographically on an agarose gel. The presence of cleaved mRNA indicates mRNA nuclease activity. Suitable controls include omission of siRNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence. Sites of siRNA-mRNA complementation are selected which result in optimal mRNA specificity and maximal mRNA cleavage.

The nucleic acid compositions of the disclosure include both unmodified siRNAs and modified siRNAs, such as crosslinked siRNA derivatives or derivatives having non-nucleotide moieties linked, for example, to their 3′ or 5′ ends. Modifying siRNA derivatives in this way may improve cellular uptake or enhance cellular targeting activities of the resulting siRNA derivative, as compared to the corresponding siRNA, and are useful for tracing the siRNA derivative in the cell, or improving the stability of the siRNA derivative compared to the corresponding siRNA.

The nucleic acid compositions of the disclosure can be unconjugated or can be conjugated to another moiety, such as a nanoparticle, to enhance a property of the compositions, e.g., a pharmacokinetic parameter such as absorption, efficacy, bioavailability and/or half-life. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present disclosure can also be labeled using any method known in the art. For instance, the nucleic acid compositions can be labeled with a fluorophore, e.g., Cy3, fluorescein, or rhodamine. The labeling can be carried out using a kit, e.g., the SILENCER^{M} siRNA labeling kit (Ambion). Additionally, the siRNA can be radiolabeled, e.g., using ³H, ³²P or another appropriate isotope.

Moreover, because RNAi is believed to progress via at least one single-stranded RNA intermediate, the skilled artisan will appreciate that ss-siRNAs (e.g., the antisense strand of a ds-siRNA) can also be designed (e.g., for chemical synthesis), generated (e.g., enzymatically generated), or expressed (e.g., from a vector or plasmid) as described herein and utilized according to the claimed methodologies. Moreover, in invertebrates, RNAi can be triggered effectively by long dsRNAs (e.g., dsRNAs about 100-1000 nucleotides in length, such as about 200-500, for example, about 250, 300, 350, 400 or 450 nucleotides in length) acting as effectors of RNAi. (Brondani et al., Proc Natl Acad Sci USA. 2001 Dec. 4; 98(25):14428-33. Epub 2001 Nov. 27.)

In certain embodiments, siRNA compounds are provided having one or any combination of the following properties: (1) fully chemically-stabilized (i.e., no unmodified 2′-OH residues); (2) asymmetry; (3) 11-21 base pair duplexes; (4) at least 50% 2′-methoxy modifications, such as 70%-100% 2′-methoxy modifications, although an alternating pattern of chemically-modified nucleotides (e.g., 2′-fluoro and 2′-methoxy modifications), are also contemplated; and (5) single-stranded, fully phosphorothioated tails of 2-8 bases. In certain embodiments, the number of phosphorothioate modifications is varied from 4 to 16 total. In certain embodiments, the number of phosphorothioate modifications is varied from 8 to 13 total.

In certain embodiments, the siRNA compounds described herein can be conjugated to a variety of targeting agents, including, but not limited to, cholesterol, docosahexaenoic acid (DHA), phenyltropanes, cortisol, vitamin A, vitamin D, N˜acetylgalactosamine (GalNac), and gangliosides.

Certain compounds of the disclosure having the structural properties described above, herein may be referred to as “hsiRNA-ASP” (hydrophobically-modified, small interfering RNA, featuring an advanced stabilization pattern). In addition, this hsiRNA-ASP pattern showed a dramatically improved distribution through several tissues, including, but not limited to, the liver, placenta, kidney, and spleen, making them accessible for therapeutic intervention.

### Design of siRNA

An siRNA of the application is a duplex made of a sense strand and complementary antisense strand, the antisense strand having sufficient complementary to a target mRNA to mediate RNAi. In certain embodiments, the siRNA has a length from about 10-50 or more nucleotides, i.e., each strand comprises 10-50 nucleotides (or nucleotide analogs). In other embodiments, the siRNA has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In certain embodiments, the strands are aligned such that there are at least 1, 2, 3, 4, 5, 6, 7, 8, 9, or 10 bases at the end of the strands, which do not align (i.e., for which no complementary bases occur in the opposing strand), such that an overhang of 1, 2, 3, 4, 5, 6, 7, 8, 9, or 10 residues occurs at one or both ends of the duplex when strands are annealed.

Usually, siRNAs can be designed by using any method known in the art, for instance, by using the following protocol:

1. The siRNA should be specific for a target sequence, e.g., a target sequence set forth in the Examples. The first strand should be complementary to the target sequence, and the other strand is substantially complementary to the first strand. (See Examples for exemplary sense and antisense strands.) Exemplary target sequences are selected from any region of the target gene that leads to potent gene silencing. Regions of the target gene include, but are not limited to, the 5′ untranslated region (5′-UTR) of a target gene, the 3′ untranslated region (3′-UTR) of a target gene, an exon of a target gene, or an intron of a target gene. Cleavage of mRNA at these sites should eliminate translation of corresponding target protein. Target sequences from other regions of the target gene are also suitable for targeting. A sense strand is designed based on the target sequence.

2. The sense strand of the siRNA is designed based on the sequence of the selected target site. In certain embodiments, the sense strand includes about 15 to 25 nucleotides, e.g., 15, 16, 17, 18, 19, 20, 21, 22, 23, 24 or 25 nucleotides. In certain embodiments, the sense strand includes 15, 16, 17, 18, 19, or 20 nucleotides. In certain embodiments, the sense strand is 15 nucleotides in length. In certain embodiments, the sense strand is 18 nucleotides in length. In certain embodiments, the sense strand is 20 nucleotides in length. The skilled artisan will appreciate, however, that siRNAs having a length of less than 15 nucleotides or greater than 25 nucleotides can also function to mediate RNAi. Accordingly, siRNAs of such length are also within the scope of the instant disclosure, provided that they retain the ability to mediate RNAi. Longer RNA silencing agents have been demonstrated to elicit an interferon or Protein Kinase R (PKR) response in certain mammalian cells which may be undesirable. In certain embodiments, the RNA silencing agents of the disclosure do not elicit a PKR response (i.e., are of a sufficiently short length). However, longer RNA silencing agents may be useful, for example, in cell types incapable of generating a PKR response or in situations where the PKR response has been down-regulated or dampened by alternative means.

The siRNA of the disclosure have sufficient complementarity with the target sequence such that the siRNA can mediate RNAi. In general, siRNA containing nucleotide sequences sufficiently complementary to a target sequence portion of the target gene to effect RISC-mediated cleavage of the target gene are contemplated. Accordingly, in a certain embodiment, the antisense strand of the siRNA is designed to have a sequence sufficiently complementary to a portion of the target. For example, the antisense strand may have 100% complementarity to the target site. However, 100% complementarity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% complementarity, between the antisense strand and the target RNA sequence is contemplated. The present application has the advantage of being able to tolerate certain sequence variations to enhance efficiency and specificity of RNAi. In one embodiment, the antisense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and the percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 7 (e.g., 2, 3, 4, 5, 6 or 7), or 1 to 4, e.g., 2, 3 or 4 nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus, in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet your criteria for evaluation.

Further general information about the design and use of siRNA may be found in “The siRNA User Guide,” available at The Max-Plank-Institut fur Biophysikalische Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log 10[Na+])+0.41(% G+C)-(600/N), where N is the number of bases in the hybrid, and [Na+] is the concentration of sodium ions in the hybridization buffer ([Na⁺] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant target mRNA), the siRNA may be incubated with target cDNA (e.g., target cDNA) in aDrosophila-based in vitro mRNA expression system. Radiolabeled with ³²P, newly synthesized target mRNAs (e.g., target mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

Modified siRNA Components

In certain aspects of the disclosure, an RNA silencing agent (or any portion thereof) of the present application, as described supra, may be modified, such that the activity of the agent is further improved. For example, the RNA silencing agents described in Section II supra, may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the present application may be substituted with a destabilizing nucleotide to enhance single nucleotide target discrimination (see U.S. application Ser. No. 11/698,689, filed Jan. 25, 2007 and U.S. Provisional Application No. 60/762,225 filed Jan. 25, 2006, both of which are incorporated herein by reference). Such a modification may be sufficient to abolish the specificity of the RNA silencing agent for a non-target mRNA (e.g. wild-type mRNA), without appreciably affecting the specificity of the RNA silencing agent for a target mRNA (e.g. gain-of-function mutant mRNA).

In certain embodiments, the RNA silencing agents of the present application are modified by the introduction of at least one universal nucleotide in the antisense strand thereof. Universal nucleotides comprise base portions that are capable of base pairing indiscriminately with any of the four conventional nucleotide bases (e.g. A, G, C, U). A universal nucleotide is contemplated because it has relatively minor effect on the stability of the RNA duplex or the duplex formed by the guide strand of the RNA silencing agent and the target mRNA. Exemplary universal nucleotides include those having an inosine base portion or an inosine analog base portion selected from the group consisting of deoxyinosine (e.g. 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In certain embodiments, the universal nucleotide is an inosine residue or a naturally occurring analog thereof.

In certain embodiments, the RNA silencing agents of the disclosure are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destablilizing nucleotide and the specificity-determining nucleotide). In RNA silencing agents having two strands or strand portions (e.g. siRNAs), the destabilizing nucleotide may be introduced in the strand or strand portion that does not contain the specificity-determining nucleotide. In certain embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the disclosure may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the present application or an siRNA produced from a shRNA) into RISC in favor of the sense strand, such that the antisense strand preferentially guides cleavage or translational repression of a target mRNA, and thus increasing or improving the efficiency of target cleavage and silencing. In certain embodiments, the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the present application may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the disclosure may be enhanced such that there is at least one mismatched base pair between the 5′ end of the first or antisense strand and the 3′ end of the sense strand portion. In certain embodiments, the mismatched base pair is selected from the group consisting of G:A, C:A, C:U, G:G, A:A, C:C and U:U. In another embodiment, the asymmetry of an RNA silencing agent of the disclosure may be enhanced such that there is at least one wobble base pair, e.g., G:U, between the 5′ end of the first or antisense strand and the 3′ end of the sense strand portion. In another embodiment, the asymmetry of an RNA silencing agent of the disclosure may be enhanced such that there is at least one base pair comprising a rare nucleotide, e.g., inosine (I). In certain embodiments, the base pair is selected from the group consisting of an I:A, I:U and I:C. In yet another embodiment, the asymmetry of an RNA silencing agent of the disclosure may be enhanced such that there is at least one base pair comprising a modified nucleotide. In certain embodiments, the modified nucleotide is selected from the group consisting of 2-amino-G, 2-amino-A, 2,6-diamino-G, and 2,6-diamino-A.

### 3) RNA Silencing Agents with Enhanced Stability

The RNA silencing agents of the present application can be modified to improve stability in serum or in growth medium for cell cultures. In order to enhance the stability, the 3′-residues may be stabilized against degradation, e.g., they may be selected such that they consist of purine nucleotides, such as adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a one aspect, the present application features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In one aspect, the present application features RNA silencing agents that are at least 80% chemically modified. In certain embodiments, the RNA silencing agents may be fully chemically modified, i.e., 100% of the nucleotides are chemically modified. In another aspect, the present application features RNA silencing agents comprising 2′-OH ribose groups that are at least 80% chemically modified. In certain embodiments, the RNA silencing agents comprise 2′-OH ribose groups that are about 80%, 85%, 90%, 95%, or 100% chemically modified.

In certain embodiments, the RNA silencing agents may contain at least one modified nucleotide analogue. The nucleotide analogues may be located at positions where the target-specific silencing activity, e.g., the RNAi mediating activity or translational repression activity is not substantially affected, e.g., in a region at the 5′-end and/or the 3′-end of the siRNA molecule. Moreover, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphodiester group connecting to adjacent ribonucleotides is replaced by a modified group, e.g., of phosphorothioate group. In exemplary sugar-modified ribonucleotides, the 2′ OH-group is replaced by a group selected from H, OR, R, halo, SH, SR, NH₂, NHR, NR₂ or ON, wherein R is C1-C6 alkyl, alkenyl or alkynyl and halo is F, Cl, Br or I.

In certain embodiments, the modifications are 2′-fluoro, 2′-amino and/or 2′-thio modifications. Modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a certain embodiment, the 2′-fluoro ribonucleotides are every uridine and cytidine. Additional exemplary modifications include 5-bromo-uridine, 5-iodo-uridine, 5-methyl-cytidine, ribothymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agent moieties of the instant disclosure. Additional modified residues include, deoxy-abasic, inosine, N3-methyl-uridine, N6,N6-dimethyl-adenosine, pseudouridine, purine ribonucleoside and ribavirin. In a certain embodiment, the 2′ moiety is a methyl group such that the linking moiety is a 2′-O-methyl oligonucleotide.

In a certain embodiment, the RNA silencing agent of the present application comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-0,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the present application comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone, which is highly resistant to nuclease digestion and imparts improved binding specificity to the molecule (Nielsen, et al., Science, (2001), 254: 1497-1500).

Also contemplated are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the present application includes RNA silencing agents having two complementary strands of nucleic acid, wherein the two strands are crosslinked. The present application also includes RNA silencing agents which are conjugated or unconjugated (e.g., at its 3′ terminus) to another moiety (e.g. a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′ OMe moiety on a U in a sense or antisense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a O with an S; (c) replacement of the U with a C5 amino linker; (d) replacement of an A with a G (sequence changes can be located on the sense strand and not the antisense strand in certain embodiments); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a O with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### Heavily Modified RNA Silencing Agents

In certain embodiments, the RNA silencing agent comprises at least 80% chemically modified nucleotides. In certain embodiments, the RNA silencing agent is fully chemically modified, i.e., 100% of the nucleotides are chemically modified.

In certain embodiments, the RNA silencing agent is 2′-O-methyl rich, i.e., comprises greater than 50% 2′-O-methyl content. In certain embodiments, the RNA silencing agent comprises at least about 55%, 60%, 65%, 70%, 75%, 80%, 85%, 90%, 95%, or 100% 2′-O-methyl nucleotide content. In certain embodiments, the RNA silencing agent comprises at least about 70% 2′-O-methyl nucleotide modifications. In certain embodiments, the RNA silencing agent comprises between about 70% and about 90% 2′-O-methyl nucleotide modifications. In certain embodiments, the RNA silencing agent is a siRNA comprising an antisense strand and sense strand. In certain embodiments, the antisense strand comprises at least about 70% 2′-O-methyl nucleotide modifications. In certain embodiments, the antisense strand comprises between about 70% and about 90% 2′-O-methyl nucleotide modifications. In certain embodiments, the sense strand comprises at least about 70% 2′-O-methyl nucleotide modifications. In certain embodiments, the sense strand comprises between about 70% and about 90% 2′-O-methyl nucleotide modifications. In certain embodiments, the sense strand comprises between 100% 2′-O-methyl nucleotide modifications.

2′-O-methyl rich RNA silencing agents and specific chemical modification patterns are further described in U.S. Patent Publication No. 2020/0087663A1 and U.S. Ser. No. 16/999,759 (filed Aug. 21, 2020), each of which is incorporated herein by reference.

### Internucleotide Linkage Modifications

In certain embodiments, at least one internucleotide linkage, intersubunit linkage, or nucleotide backbone is modified in the RNA silencing agent. In certain embodiments, all of the internucleotide linkages in the RNA silencing agent are modified. In certain embodiments, the modified intemucleotide linkage comprises a phosphorothioate intemucleotide linkage. In certain embodiments, the RNA silencing agent comprise 1, 2, 3, 4, 5, 6, 7, 8, 9, 10, 11, 12, 13, 14, 15, 16, 17, 18, 19, 20, 21, 22, 23, 24, or 25 phosphorothioate intemucleotide linkages. In certain embodiments, the RNA silencing agent comprises 4-16 phosphorothioate intemucleotide linkages. In certain embodiments, the RNA silencing agent comprises 8-13 phosphorothioate internucleotide linkages. In certain embodiments, the RNA silencing agent is a siRNA comprising an antisense strand and a sense strand, each comprising a 5′ end and a 3′ end. In certain embodiments, the nucleotides at positions 1 and 2 from the 5′ end of sense strand are connected to adjacent ribonucleotides via phosphorothioate intemucleotide linkages. In certain embodiments, the nucleotides at positions 1 and 2 from the 3′ end of sense strand are connected to adjacent ribonucleotides via phosphorothioate intemucleotide linkages. In certain embodiments, the nucleotides at positions 1 and 2 from the 5′ end of antisense strand are connected to adjacent ribonucleotides via phosphorothioate intemucleotide linkages. In certain embodiments, the nucleotides at positions 1-2 to 1-8 from the 3′ end of antisense strand are connected to adjacent ribonucleotides via phosphorothioate intemucleotide linkages. In certain embodiments, the nucleotides at positions 1-2, 1-3, 1-4, 1-5, 1-6, 1-7, or 1-8 from the 3′ end of antisense strand are connected to adjacent ribonucleotides via phosphorothioate intemucleotide linkages. In certain embodiments, the nucleotides at positions 1-2 to 1-7 from the 3′ end of antisense strand are connected to adjacent ribonucleotides via phosphorothioate internucleotide linkages.

In one aspect, the disclosure provides a modified oligonucleotide, said oligonucleotide having a 5′ end, a 3′ end, that is complementary to a target, wherein the oligonucleotide comprises a sense and antisense strand, and at least one modified intersubunit linkage of Formula (I):

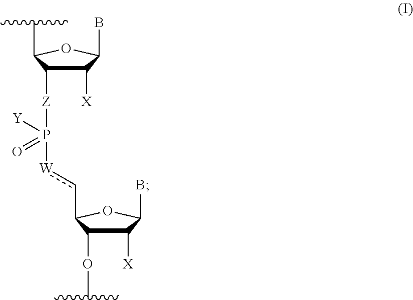

wherein:
   B is a base pairing moiety;
   W is selected from the group consisting of O, OCH₂, OCH, CH₂, and CH;
   X is selected from the group consisting of halo, hydroxy, and C₁₋₆ alkoxy;
   Y is selected from the group consisting of O⁻, OH, OR, NH⁻, NH₂, S⁻, and SH;
   Z is selected from the group consisting of O and CH₂;
   R is a protecting group; and
[symbol] is an optional double bond.

In an embodiment of Formula (I), when W is CH, [symbol] is a double bond.

In an embodiment of Formula (I), when W selected from the group consisting of O, OCH₂, OCH, CH₂, [symbol] is a single bond.

In an embodiment of Formula (I), when Y is O⁻, either Z or W is not O.

In an embodiment of Formula (I), Z is CH₂ and W is CH₂. In another embodiment, the modified intersubunit linkage of Formula (I) is a modified intersubunit linkage of Formula (II):

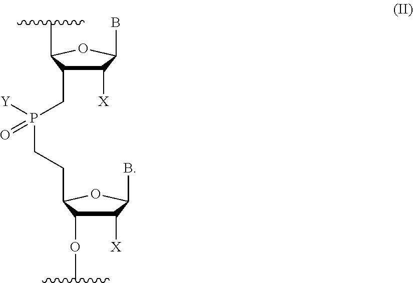

In an embodiment of Formula (I), Z is CH₂ and W is O. In another embodiment, wherein the modified intersubunit linkage of Formula (I) is a modified intersubunit linkage of Formula (III):

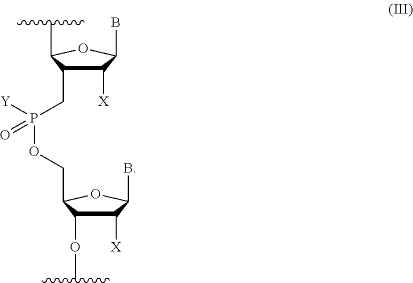

In an embodiment of Formula (I), Z is O and W is CH₂. In another embodiment, the modified intersubunit linkage of Formula (I) is a modified intersubunit linkage of Formula (IV):

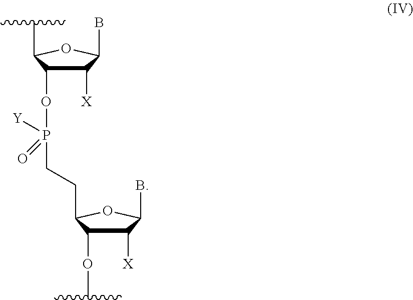

In an embodiment of Formula (I), Z is O and W is CH. In another embodiment, the modified intersubunit linkage of Formula (I) is a modified intersubunit linkage of Formula V:

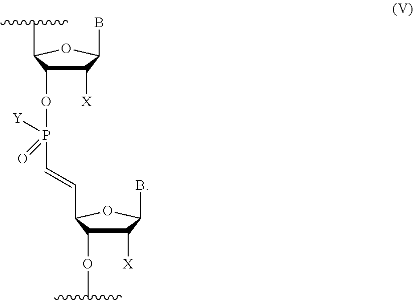

In an embodiment of Formula (I), Z is O and W is OCH₂. In another embodiment, the modified intersubunit linkage of Formula (I) is a modified intersubunit linkage of Formula VI:

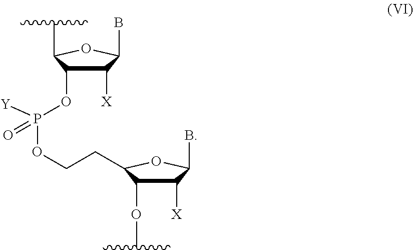

In an embodiment of Formula (I), Z is CH₂ and W is CH. In another embodiment, the modified intersubunit linkage of Formula (I) is a modified intersubunit linkage of Formula VII:

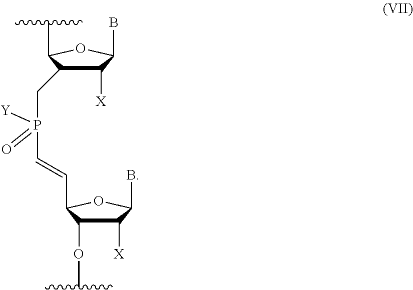

In an embodiment of Formula (I), the base pairing moiety B is selected from the group consisting of adenine, guanine, cytosine, and uracil.

In an embodiment, the modified oligonucleotide is incorporated into siRNA, said modified siRNA having a 5′ end, a 3′ end, that is complementary to a target, wherein the siRNA comprises a sense and antisense strand, and at least one modified intersubunit linkage of any one or more of Formula (I), Formula (II), Formula (III), Formula (IV), Formula (V), Formula (VI), or Formula (VII).

In an embodiment, the modified oligonucleotide is incorporated into siRNA, said modified siRNA having a 5′ end, a 3′ end, that is complementary to a target and comprises a sense and antisense strand, wherein the siRNA comprises at least one modified intersubunit linkage is of Formula VIII:

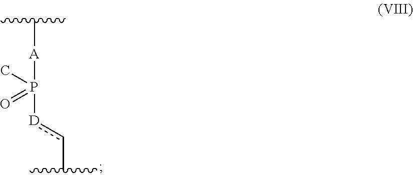

wherein:
   D is selected from the group consisting of O, OCH₂, OCH, CH₂, and CH;
   C is selected from the group consisting of O⁻, OH, OR¹, NH⁻, NH₂, S⁻, and SH;
   A is selected from the group consisting of O and CH₂;
R¹ is a protecting group;
   [symbol] is an optional double bond; and
   the intersubunit is bridging two optionally modified nucleosides.

In an embodiment, when C is O, either A or D is not O.

In an embodiment, D is CH₂. In another embodiment, the modified intersubunit linkage of Formula VIII is a modified intersubunit linkage of Formula (IX):

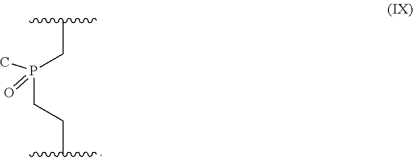 [SMILES: *CCP(C)(=O)C*]

In an embodiment, D is O. In another embodiment, the modified intersubunit linkage of Formula VIII is a modified intersubunit linkage of Formula (X):

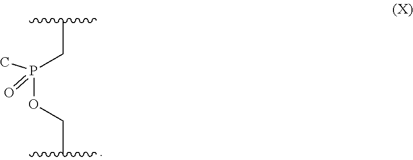 [SMILES: *COP(C)(=O)C*]

In an embodiment, D is CH₂. In another embodiment, the modified intersubunit linkage of Formula (VIII) is a modified intersubunit linkage of Formula (XI):

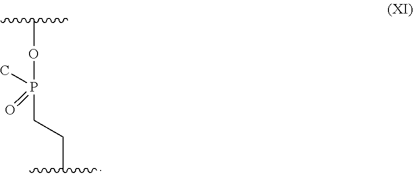 [SMILES: *CCP(C)(=O)O*]

In an embodiment, D is CH. In another embodiment, the modified intersubunit linkage of Formula VIII is a modified intersubunit linkage of Formula (XII):

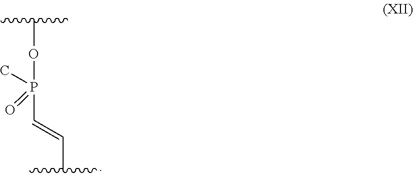 [SMILES: */C=C/P(C)(=O)O*]

In another embodiment, the modified intersubunit linkage of Formula (VII) is a modified intersubunit linkage of Formula (XIV):

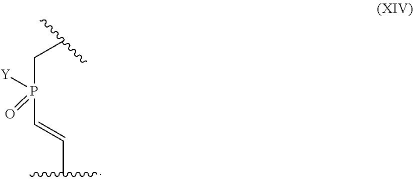

In an embodiment, D is OCH₂. In another embodiment, the modified intersubunit linkage of Formula (VII) is a modified intersubunit linkage of Formula (XIII):

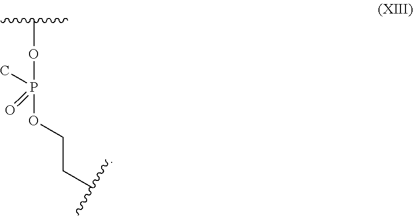 [SMILES: *CCOP(C)(=O)O*]

In another embodiment, the modified intersubunit linkage of Formula (VII) is a modified intersubunit linkage of Formula (XXa):

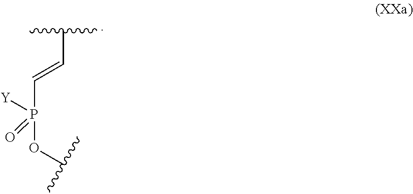

In an embodiment of the modified siRNA linkage, each optionally modified nucleoside is independently, at each occurrence, selected from the group consisting of adenosine, guanosine, cytidine, and uridine.

In certain exemplary embodiments of Formula (I), W is O. In another embodiment, W is CH₂. In yet another embodiment, W is CH.

In certain exemplary embodiments of Formula (I), X is OH. In another embodiment, X is OCH₃. In yet another embodiment, X is halo.

In a certain embodiment of Formula (I), the modified siRNA does not comprise a 2′-fluoro substituent.

In an embodiment of Formula (I), Y is O⁻. In another embodiment, Y is OH. In yet another embodiment, Y is OR. In still another embodiment, Y is NH⁻. In an embodiment, Y is NH₂. In another embodiment, Y is S⁻. In yet another embodiment, Y is SH.

In an embodiment of Formula (I), Z is O. In another embodiment, Z is CH₂.

In an embodiment, the modified intersubunit linkage is inserted on position 1-2 of the antisense strand. In another embodiment, the modified intersubunit linkage is inserted on position 6-7 of the antisense strand. In yet another embodiment, the modified intersubunit linkage is inserted on position 10-11 of the antisense strand. In still another embodiment, the modified intersubunit linkage is inserted on position 19-20 of the antisense strand. In an embodiment, the modified intersubunit linkage is inserted on positions 5-6 and 18-19 of the antisense strand.

In an exemplary embodiment of the modified siRNA linkage of Formula (VIII), C is O⁻. In another embodiment, C is OH. In yet another embodiment, C is OR¹. In still another embodiment, C is NH⁻. In an embodiment, C is NH₂. In another embodiment, C is S⁻. In yet another embodiment, C is SH.

In an exemplary embodiment of the modified siRNA linkage of Formula (VIII), A is O. In another embodiment, A is CH₂. In yet another embodiment, C is OR¹. In still another embodiment, C is NH⁻. In an embodiment, C is NH₂. In another embodiment, C is S⁻. In yet another embodiment, C is SH.

In a certain embodiment of the modified siRNA linkage of Formula (VIII), the optionally modified nucleoside is adenosine. In another embodiment of the modified siRNA linkage of Formula (VIII), the optionally modified nucleoside is guanosine. In another embodiment of the modified siRNA linkage of Formula (VIII), the optionally modified nucleoside is cytidine. In another embodiment of the modified siRNA linkage of Formula (VIII), the optionally modified nucleoside is uridine.

In an embodiment of the modified siRNA linkage, wherein the linkage is inserted on position 1-2 of the antisense strand. In another embodiment, the linkage is inserted on position 6-7 of the antisense strand. In yet another embodiment, the linkage is inserted on position 10-11 of the antisense strand. In still another embodiment, the linkage is inserted on position 19-20 of the antisense strand. In an embodiment, the linkage is inserted on positions 5-6 and 18-19 of the antisense strand.

In certain embodiments of Formula (I), the base pairing moiety B is adenine. In certain embodiments of Formula (I), the base pairing moiety B is guanine. In certain embodiments of Formula (I), the base pairing moiety B is cytosine. In certain embodiments of Formula (I), the base pairing moiety B is uracil.

In an embodiment of Formula (I), W is O. In an embodiment of Formula (I), W is CH₂. In an embodiment of Formula (I), W is CH.

In an embodiment of Formula (I), X is OH. In an embodiment of Formula (I), X is OCH₃. In an embodiment of Formula (I), X is halo.

In an exemplary embodiment of Formula (I), the modified oligonucleotide does not comprise a 2′-fluoro substituent.

In an embodiment of Formula (I), Y is O. In an embodiment of Formula (I), Y is OH. In an embodiment of Formula (I), Y is OR. In an embodiment of Formula (I), Y is NH—. In an embodiment of Formula (I), Y is NH₂. In an embodiment of Formula (I), Y is S⁻. In an embodiment of Formula (I), Y is SH.

In an embodiment of Formula (I), Z is O. In an embodiment of Formula (I), Z is CH₂.

In an embodiment of the Formula (I), the linkage is inserted on position 1-2 of the antisense strand. In another embodiment of Formula (I), the linkage is inserted on position 6-7 of the antisense strand. In yet another embodiment of Formula (I), the linkage is inserted on position 10-11 of the antisense strand. In still another embodiment of Formula (I), the linkage is inserted on position 19-20 of the antisense strand. In an embodiment of Formula (I), the linkage is inserted on positions 5-6 and 18-19 of the antisense strand.

Modified intersubunit linkages are further described in WO 2020/198509 and U.S. Ser. No. 63/000,328 (filed Mar. 26, 2020), each of which is incorporated herein by reference.

### 4) Conjugated Functional Moieties

In other embodiments, RNA silencing agents may be modified with one or more functional moieties. A functional moiety is a molecule that confers one or more additional activities to the RNA silencing agent. In certain embodiments, the functional moieties enhance cellular uptake by target cells (e.g., neuronal cells). Thus, the disclosure includes RNA silencing agents which are conjugated or unconjugated (e.g., at its 5′ and/or 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a certain embodiment, the functional moiety is a hydrophobic moiety. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and Lithocholic acid (LCA). In a certain embodiment, the fatty acid selected from the group consisting of Eicosapentaenoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an RNA silencing agent of disclosure is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the 3′ end of the sense strand. In certain embodiments, the lipophilic moiety is selected from the group consisting of cholesterol, vitamin E, vitamin K, vitamin A, folic acid, a cationic dye (e.g., Cy3). In an exemplary embodiment, the lipophilic moiety is cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine.

In certain embodiments, the functional moieties may comprise one or more ligands tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These can be located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., 0-phenanthroline), a polyamine, a tripeptide (e.g., lys-tyr-lys tripeptide), or a metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine, has an increased affinity for the HIV Rev-response element (RRE). In some embodiments, the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, either directly or indirectly, via an intervening tether, to a ligand-conjugated carrier. In certain embodiments, the coupling is through a covalent bond. In certain embodiments, the ligand is attached to the carrier via an intervening tether. In certain embodiments, a ligand alters the distribution, targeting or lifetime of an RNA silencing agent into which it is incorporated. In certain embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine (GalNAc) or derivatives thereof, N-acetyl-glucosamine, multivalent mannose, multivalent fucose, glycosylated poly aminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g., cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, 03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP or AP. In certain embodiments, the ligand is GalNAc or a derivative thereof.

In certain embodiments, the GalNAc is represented by the formula below:

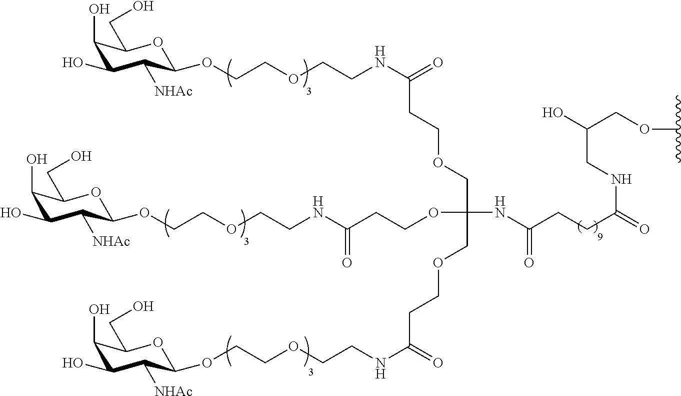

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-kB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNF Q), interleukin-1 beta, or gamma interferon. In one aspect, the ligand is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a certain embodiment, the lipid based ligand binds HSA. A lipid-based ligand can bind HSA with a sufficient affinity such that the conjugate will be distributed to a non-kidney tissue. However, it is contemplated that the affinity not be so strong that the HSA-ligand binding cannot be reversed. In another embodiment, the lipid based ligand binds HSA weakly or not at all, such that the conjugate will be distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These can be useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

In certain embodiments, the functional moiety is linked to the 5′ end and/or 3′ end of the RNA silencing agent of the disclosure. In certain embodiments, the functional moiety is linked to the 5′ end and/or 3′ end of an antisense strand of the RNA silencing agent of the disclosure. In certain embodiments, the functional moiety is linked to the 5′ end and/or 3′ end of a sense strand of the RNA silencing agent of the disclosure. In certain embodiments, the functional moiety is linked to the 3′ end of a sense strand of the RNA silencing agent of the disclosure.

In certain embodiments, the functional moiety is linked to the RNA silencing agent by a linker. In certain embodiments, the functional moiety is linked to the antisense strand and/or sense strand by a linker. In certain embodiments, the functional moiety is linked to the 3′ end of a sense strand by a linker. In certain embodiments, the linker is a cleavable linker. In certain embodiments, the cleavable linker comprises a phosphodiester linkage, a disulfide linkage, an acid-labile linkage, or a photocleavable linkage.

In certain embodiments, the cleavable linker comprises a dTdT dinucleotide with phosphodiester intemucleotide linkages.

In certain embodiments, the acid-labile linkage comprises a 0-thiopropionate linkage or a carboxydimethylmaleic anhydride (CDM) linkage.

In certain embodiments, the linker comprises a divalent or trivalent linker. In certain embodiments, the linker comprises an ethylene glycol chain, an alkyl chain, a peptide, RNA, DNA, a phosphodiester, a phosphorothioate, a phosphoramidate, an amide, a carbamate, or a combination thereof. In certain embodiments, the divalent or trivalent linker is selected from:

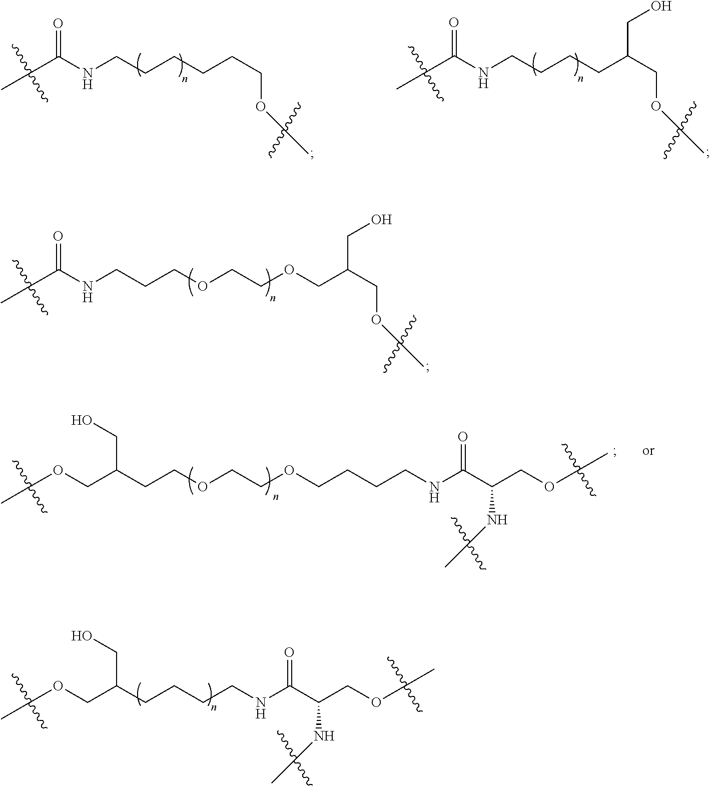

or wherein n is 1, 2, 3, 4, or 5.

In certain embodiments, the linker further comprises a phosphodiester or phosphodiester derivative. In certain embodiments, the phosphodiester or phosphodiester derivative is selected from the group consisting of:

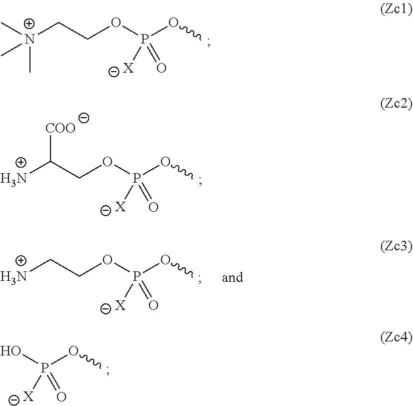

wherein X is O, S or BH₃.

The various functional moieties of the disclosure and means to conjugate them to RNA silencing agents are described in further detail in WO2017/030973A1 and WO2018/031933A2, incorporated herein by reference.

### III. siRNASO—Antisense Oligonucleotide Component

As used herein, the term “antisense oligonucleotide” refers to an oligonucleotide molecule, which is capable of binding to RNA inside cells by Watson-Crick base pairing. Depending on the sequence and chemistry of the antisense oligonucleotide, this interaction can lead to silencing of a target gene (i.e. reducing the level of expression of mature mRNA and/or protein from that gene) or activation of a target gene (i.e. increasing the level of expression of mature mRNA and/or protein from that gene). The antisense oligonucleotides of the present disclosure are focused on activating gene expression, which can be done utilizing different mechanisms. Some antisense oligonucleotides are designed to recruit RNase H to cleave their target RNAs. RNase H is a family of non-sequence-specific endonuclease enzymes that catalyze the cleavage of RNA in an RNA/DNA substrate via a hydrolytic mechanism. In certain embodiments, the antisense oligonucleotides of the disclosure trigger RNase H-mediated cleavage of a pre-mRNA target, which can be compatible with activation of overall target gene expression. Other antisense oligonucleotides, called steric blockers, are designed not to elicit cleavage of their targets but to block interactions with cellular factors. For example, these cellular factors could modulate splicing, block interactions of noncoding RNAs or of RNA-binding proteins, stabilize mRNA to prolong its half-life, or increase the efficiency of translation of an mRNA.

As used herein, the term “heteroduplex oligonucleotide” or “HDO” refers to an antisense oligonucleotide-based compound that comprises an antisense oligonucleotide as described herein and a complementary oligonucleotide, annealed to said antisense oligonucleotide, thereby producing a duplex (the HDO). The HDO complementary oligonucleotide may comprise any of the chemical modifications employed in the ASO. HDOs are described in further detail in Nishina et al. (Nature Communications volume 6, Article number: 7969. 2015), WO2014192310A1, and WO2014203518A1 each of which is incorporated herein by reference.

Antisense oligonucleotides designed to recruit RNase H are often designed as “gapmers.” The term “gapmer” means a chimeric antisense oligonucleotide in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.” “Chimeric antisense oligonucleotide” means an antisense oligonucleotide that has at least two chemically distinct regions.

The term “antisense activity” means any detectable or measurable activity attributable to the hybridization of an antisense compound to its target nucleic acid. In some embodiments, antisense activity is an increase in the amount or expression of a target nucleic acid or protein encoded by such target nucleic acid. “Antisense compound” means an oligomeric compound that is capable of undergoing hybridization to a target nucleic acid through hydrogen bonding. As used herein, “antisense oligonucleotide” means a single-stranded oligonucleotide having a nucleobase sequence that permits hybridization to a corresponding region or segment of a target nucleic acid.

The term “antisense inhibition” means reduction of target nucleic acid levels in the presence of an antisense oligonucleotide having a sequence that is sufficiently complementary to a target nucleic acid compared to target nucleic acid levels in the absence of the antisense compound. A target nucleic acid can be any nucleic acid.

The term “target-recognition sequence” refers to the portion of an antisense compound that recognizes a target nucleic acid. The target-recognition sequence has a nucleobase sequence that permits hybridization to a corresponding region or segment of a target nucleic acid.

The term “conserved region” refers to a portion, or portions, of a nucleic acid sequence that is conserved, i.e. a portion, or portions of the nucleic acid sequence having a similar or identical sequence across species. A conserved region can be computationally identified, e.g., using any sequence alignment software available in the art.

As used herein, a “region of complementarity” refers to a portion of the antisense oligonucleotide that is complementary to the target. For example, but in no way limiting, an 18-nucleotide long antisense oligonucleotide can comprise a contiguous 12-nucleotide portion that is complementary to the target transcript. In certain embodiments, the antisense oligonucleotide is complementary to the target transcript over the full length of the antisense oligonucleotide.

In some embodiments, an antisense compound of the present disclosure is an antisense oligonucleotide. Chimeric antisense oligonucleotides typically contain at least one region modified so as to confer increased resistance to nuclease degradation, increased cellular uptake, increased binding affinity for the target nucleic acid, and/or increased activity. A second region of a chimeric antisense compound can optionally serve as a substrate for the cellular endonuclease RNase H, which cleaves the RNA strand of an RNA:DNA duplex. In some embodiments, an antisense compound of the present disclosure is a chimeric antisense oligonucleotide having a gapmer motif. In a gapmer, an internal region having a plurality of nucleotides that supports RNase H cleavage is positioned between external regions having a plurality of nucleotides that are chemically distinct from the nucleosides of the internal region.

In some embodiments, the present disclosure provides an antisense oligonucleotide having a target-recognition sequence that is sufficiently complementary to a target transcript or portion thereof, to direct cleavage of the target transcript by RNase H. The target-recognition sequence of the antisense oligonucleotide can be the full length of the antisense oligonucleotide, or a portion thereof. In some embodiments, the antisense oligonucleotide comprises a gapmer motif.

In the case of an antisense compound having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include R-D-ribonucleosides, β-D-deoxyribonucleosides, 2′-modified nucleosides (such 2′-modified nucleosides can include 2′-MOE, and 2′-O—CH₃ (i.e., OMe), among others), and bicyclic sugar modified nucleosides (such bicyclic sugar modified nucleosides can include those having a 4′-(CH₂)n-O-2′ bridge, where n=1 or n=2). In some embodiments, the wing segments of the gapmer contain one or more tricyclo-DNA (tcDNA) modifications. In some embodiments, each distinct region comprises uniform sugar moieties. In some embodiments, each wing segment comprises a mixture of different nucleotide modifications. For example, in one embodiment, a LNA modification and a 2′-MOE modification could be used in combination for one antisense compound. In one embodiment, a LNA modification and a 2′-O-Methyl modification could be used in combination for one antisense compound. In one embodiment, a LNA modification and a 2′-deoxy modification could be used in combination for one antisense compound. In one embodiment, a LNA modification and a tricyclo-DNA modification could be used in combination for one antisense compound. In one embodiment, a 2′-MOE modification and a tricyclo-DNA modification could be used in combination for one antisense compound.

The gapmer motif can be described using the formula “A-B-C”, where “A” represents the length of the 5′ wing region, “B” represents the length of the gap region, and “C” represents the length of the 3′ wing region. As such, in some embodiments, an antisense oligonucleotide of the present disclosure has the formula:

A-B-C.

As used herein, a gapmer described as “A-B-C” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment.

In some embodiments, the 5′ wing region represented by “A” comprises from about 0 to about 8 modified nucleotides, e.g., from about 1 to about 6 modified nucleotides. For example, the 5′ wing region represented by “A” can be 0, 1, 2, 3, 4, 5, 6, 7, or 8 nucleotides in length. In some embodiments, the 3′ wing region represented by “C” comprises about 0 to about 8 modified nucleotides, e.g., from about 1 to about 6 modified nucleotides. For example, the 3′ wing region represented by “C” can be 0, 1, 2, 3, 4, 5, 6, 7, or 8 nucleotides in length. In some embodiments, “A” and “C” are the same, in some embodiments, they are different.

In some embodiments, the gap region represented by “B” comprises from about 6 to about 18 DNA nucleotides and/or DNA-like nucleotides, e.g., from about 6 to about 12 DNA nucleotides and/or DNA-like nucleotides. For example, the gap region represented by “B” can be 6, 7, 8, 9, 10, 11, 12, 13, 14, 15, 16, 17, or 18 DNA nucleotides and/or DNA-like nucleotides in length. Thus, an antisense oligonucleotide of the present disclosure having a target-recognition sequence with the formula “A-B-C” include, but are not limited to the following gapmer formats, for example 1-10-1 (i.e., one nucleotide—ten nucleotides—one nucleotide), 1-10-1, 1-11-1, 1-12-1, 2-8-2, 2-9-2, 2-10-2, 2-11-2, 2-12-2, 3-6-3, 3-7-3, 3-8-3, 3-9-3, 3-10-3, 3-11-3, 3-12-3, 4-6-4, 4-7-4, 4-8-4, 4-9-4, 4-10-4, 4-11-4, 4-12-4, 5-6- 5, 5-7-5, 5-8-5, 5-9-5, 5-10-5, 5-11-5, 5-12-5, 6-6-6, 6-7-6, 6-8-6, 6-9-6, 6-10-6, 6-11-6, or 6- 12-6. The wings can also be of different lengths, such as 1-10-6, 3-9-5, 7-9-2, 4-10-5, or other asymmetric combinations of wing lengths flanking a central DNA gap. In certain embodiments, the gapmer of “A-B-C” is at least 12 nucleotides in length. In certain embodiments, “B” is at least 6 nucleotides in length. A person of skill in the art will be able to identify additional asymmetric combinations of wing lengths.

In certain embodiments, antisense compounds targeted to a target nucleic acid possess a 5-9-4 gapmer format. In some embodiments, the antisense compound is an antisense oligonucleotide having a target-recognition sequence with the 5-9-4 format that is sufficiently complementary to a target transcript, or a portion thereof, to direct cleavage of the target transcript by RNase H. In some embodiments, the target-recognition sequence has the formula “A-B-C”, wherein “A” comprises about 2 to 6 modified nucleotides, “B” comprises about 6 to 12 DNA nucleotides and/or DNA-like nucleotides, and “C” comprises about 2 to 6 modified nucleotides. In some embodiments, the target-recognition sequence has the formula “A-B-C”, wherein “A” comprises 5 modified nucleotides, “B” comprises 9 DNA nucleotides and/or DNA-like nucleotides, and “C” comprises 4 modified nucleotides. In some embodiments, the target-recognition sequence has the formula “A-B-C”, wherein “A” comprises 2 to 6 2′-O-(2-methoxyethyl) (MOE) modified nucleotides, “B” comprises 6 to 12 DNA nucleotides and/or DNA-like nucleotides, and “C” comprises 2 to 6 2′-O-(2-methoxyethyl) (MOE) modified nucleotides. In some embodiments, the target-recognition sequence has the formula “A-B-C”, wherein “A” comprises 5 2′-O-(2-methoxyethyl) (MOE) modified nucleotides, “B” comprises 9 DNA nucleotides and/or DNA-like nucleotides, and “C” comprises 4 2′-O-(2-methoxyethyl) (MOE) modified nucleotides.

In some embodiments, antisense compounds that target a target nucleic acid possess a “wingmer” motif. The wingmer motif can be described using the formula “X-Y” or “Y-X”, where “X” represents the length of the wing region, and “Y” represents the length of the gap region. As such, in some embodiments, an antisense oligonucleotide of the present disclosure has the formula:

X-Y, or

Y-X.

As used herein, a wingmer described as “X-Y” or “Y-X” has a configuration such that the gap segment is positioned immediately adjacent to the wing segment. Thus, no intervening nucleotides exist between the wing segment and the gap segment. Non-limiting examples of wingmer configurations of an antisense compound of the present disclosure include, e.g., 1-15, 1-17, 1-19, 2-15, 2-17, 2-19, 2-22, 3-13, 3-17, 3-20, 3-21, 3-22, 4-12, 4-14, 4-16, 4-18, 4-19, 4-21, 5-11, 5-13, 5-14, 5-15, 5-16, 5-18, or 5-20.

In some embodiments, antisense compounds targeted to a target nucleic acid possess a gap-widened motif. As used herein, “gap-widened” refers to an antisense compound having a gap segment of 12 or more contiguous DNA nucleotides and/or DNA-like nucleotides adjacent to a wing region. In the case of a gap-widened gapmer, the gapmer comprises a gap region having 12 or more contiguous DNA nucleotides and/or DNA-like nucleotides positioned between and immediately adjacent to the 5′ and 3′ wing segments. In the case of a gap-widened wingmer, the wingmer comprises a gap region having 12 or more contiguous DNA nucleotides and/or DNA-like nucleotides positioned immediately adjacent to the wing segment.

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidates, and phosphorothioates. Methods of preparation of phosphorous-containing and non-phosphorous-containing linkages are well known.

In certain embodiments, antisense compounds targeted to a target nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

Antisense compounds of the disclosure can optionally contain one or more nucleosides wherein the sugar group has been modified. Such sugar-modified nucleosides can impart enhanced nuclease stability, increased binding affinity or some other beneficial biological property to the antisense compounds. In certain embodiments, nucleosides comprise a chemically modified ribofuranose ring moieties. Examples of chemically modified ribofuranose rings include without limitation, addition of substituent groups (including 5′ and 2′ substituent groups, bridging of ring atoms to form bicyclic nucleic acids (BNA), replacement of the ribosyl ring oxygen atom with S, N(R), or C(R¹)(R²) (R, R¹, R²═H, C₁-C₁₂ alkyl or a protecting group) and combinations thereof. Examples of chemically modified sugars include 2′-F-5′-methyl substituted nucleoside (see WO 2008/101157 for other disclosed 5′,2′-bis substituted nucleosides) or replacement of the ribosyl ring oxygen atom with S with further substitution at the 2′-position (see U.S. Patent Application US20050130923) or alternatively 5′-substitution of a BNA (see WO 2007/134181, wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F (i.e., 2′-fluoro), 2′-OCH₃ (i.e., 2′-O-methyl) and 2′-O(CH₂)₂OCH₃ (i.e., 2′-O-methoxyethyl) substituent groups. The substituent at the 2′ position can also be selected from allyl, amino, azido, thio, 0-allyl, 0-C₁-C₁₀ alkyl, OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. 2′-modified nucleotides are useful in the present disclosure, for example, 2′-O-methyl RNA, 2′-0-methoxyethyl RNA, 2′-fluoro RNA, and others envisioned by one of ordinary skill in the art.

Examples of bicyclic nucleic acids (BNAs) include without limitation nucleosides comprising a bridge between the 4′ and the 2′ ribosyl ring atoms. A BNA comprising a bridge between the 4′ and 2′ ribosyl ring atoms can be referred to as a locked nucleic acid (LNA), and is often referred to as inaccessible RNA. As used herein, the term “locked nucleotide” or “locked nucleic acid (LNA)” comprises nucleotides in which the 2′ deoxy ribose sugar moiety is modified by introduction of a structure containing a heteroatom bridging from the 2′ to the 4′ carbon atoms. The term “non-locked nucleotide” comprises nucleotides that do not contain a bridging structure in the ribose sugar moiety. Thus, the term comprises DNA and RNA nucleotide monomers (phosphorylated adenosine, guanosine, uridine, cytidine, deoxyadenosine, deoxyguanosine, deoxythymidine, deoxycytidine) and derivatives thereof as well as other nucleotides having a 2′-deoxy-erythro-pentofuranosyl sugar moiety or a ribo-pentofuranosyl moiety. In certain embodiments, antisense compounds provided herein include one or more BNA nucleosides wherein the bridge comprises one of the formulas: 4′-(CH₂)—O-2′ (LNA); 4′-(CH₂)—S-2′; 4′-(CH₂)—O-2′ (LNA); 4′-(CH₂)₂—O-2′ (ENA); 4′-C(CH₃)₂—O-2′ (see PCT/US2008/068922); 4′-CH(CH₃)—O-2′ and 4′-CH(CH₂OCH₃)—O-2′ (see U.S. Pat. No. 7,399,845, issued on Jul. 15, 2008); 4′-CH₂—N(OCH₃)-2′ (see PCT/US2008/064591); 4′-CH₂—O—N(CH₃)-2′ (see published U.S. Patent Application US2004-0171570, published Sep. 2, 2004); 4′-CH₂—N(R)—O-2′ (see U.S. Pat. No. 7,427,672, issued on Sep. 23, 2008); 4′-CH₂—C(CH₃)-2′ and 4′-CH₂—C(═CH₂)-2′ (see PCT/US2008/066154); and wherein R is, independently, H, C₁-C₁₂ alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226).

In some embodiments, antisense compounds provided herein include one or more 2′, 4′-constrained nucleotides. For example, antisense compounds provided by the present disclosure include those having one or more constrained ethyl (cEt) or constrained methoxyethyl (cMOE) nucleotides. In some embodiments, antisense compounds provided herein are antisense oligonucleotides comprising one or more constrained ethyl (cEt) nucleotides. The terms “constrained ethyl” and “ethyl-constrained” are used interchangeably.

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

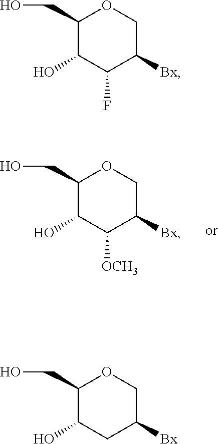

In certain embodiments, antisense oligonucleotides may comprise morpholino rings joined by phosphorodiamidate linkages. These may be referred to as PMO oligomers or phosphorodiamidate morpholino oligomers. In certain such embodiments, the backbone of these oligonucleotides may be uncharged. In other embodiments, one or more of the phosphorodiamidate linkages may comprise a charged moiety.

Many other bicyclo and tricyclo sugar surrogate ring systems are also known in the art that can be used to modify nucleosides for incorporation into antisense compounds (see for example review article: Leumann, J. C, Bioorganic & Medicinal Chemistry, 2002, 10, 841-854; Ito, K. R.; Obika, S., Recent Advances in Medicinal Chemistry of Antisense Oligonucleotides. In Comprehensive Medicinal Chemistry, 3rd edition, Elsevier: 2017). Such ring systems can undergo various additional substitutions to enhance activity.

Methods for the preparations of modified sugars are well known to those skilled in the art. In nucleotides having modified sugar moieties, the nucleobase moieties (natural, modified or a combination thereof) are maintained for hybridization with an appropriate nucleic acid target.

In certain embodiments, antisense compounds targeted to a target nucleic acid comprise one or more kinds of modified nucleotides. In one embodiment, antisense compounds targeted to a target nucleic acid comprise 2′-modified nucleotides. In one embodiment, antisense compounds targeted to a target nucleic acid comprise a 2′-O-methyl RNA, a 2′-O-methoxyethyl RNA, or a 2′-fluoro RNA. In one embodiment, antisense compounds targeted to a target nucleic acid comprise tricyclo-DNA (tcDNA). Tricyclo-DNA belongs to a class of constrained DNA analogs that display improved hybridizing capacities to complementary RNA, see, e.g., Ittig et al., Nucleic Acids Res. 32:346-353 (2004); Ittig et al., Prague, Academy of Sciences of the Czech Republic. 7:21-26 (Coll. Symp. Series, Hocec, M., 2005); Ivanova et al., Oligonucleotides 17:54-65 (2007); Renneberg et al., Nucleic Acids Res. 30:2751-2757 (2002); Renneberg et al., Chembiochem. 5:1114-1118 (2004); and Renneberg et al., JACS. 124:5993-6002 (2002). In one embodiment, antisense compounds targeted to a target nucleic acid comprise a locked nucleotide, an ethyl-constrained nucleotide, or an alpha-L-locked nucleic acid. Various alpha-L-locked nucleic acids are known by those of ordinary skill in the art, and are described in, e.g., Sorensen et al., J. Am. Chem. Soc. (2002) 124(10):2164-2176.

In certain embodiments, the antisense compounds targeting a target nucleic acid are fully chemically modified, i.e., every nucleotide is chemically modified. In certain embodiments, every nucleotide comprises a 2′-O-(2-methoxyethyl) (MOE) modification. In certain embodiments, every nucleotide comprises a tricyclo-DNA modification. In certain embodiments, the antisense compounds targeting a target nucleic acid comprise a mixture of tricyclo-DNA modifications and 2′-O-(2-methoxyethyl) (MOE) modifications, wherein every nucleotide of the antisense compounds is either tcDNA or MOE.

In certain embodiments, antisense compounds targeted to a target nucleic acid comprise one or more modified nucleotides having modified sugar moieties. In some embodiments, the modified nucleotide is a locked nucleotide. In certain embodiments, the locked nucleotides are arranged in a gapmer motif, e.g. a 3-9-3 gapmer format wherein 9 non-locked nucleotides are flanked by 3 locked nucleotides on each side.

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo such as 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are useful for increasing the binding affinity of antisense compounds include 5-substituted pyrimidines, 6-azapyrimidines and N-2, N-6 and 0-6 substituted purines, including 2 aminopropyladenine, 5-propynyluracil and 5-propynylcytosine.

In certain embodiments, antisense compounds targeted to a target nucleic acid comprise one or more modified nucleotides having modified sugar moieties. In some embodiments, the modified nucleotide is a locked nucleotide. In certain embodiments, the locked nucleotides are arranged in a gapmer motif, e.g. a 3-9-3 gapmer format wherein 9 non-locked nucleotides are flanked by 3 locked nucleotides on each side. In certain embodiments, antisense compounds targeted to a target nucleic acid comprise one or more modified nucleotides. In some embodiments, the modified nucleotide is 5-methylcytosine. In certain embodiments, each cytosine is a 5-methylcytosine. In some embodiments, the modified nucleotide is a 2′-O-(2-methoxyethyl) (MOE) modified nucleotide. In certain embodiments, the 2′-O-(MOE) modified nucleotides are arranged in a gapmer motif, e.g. a 5-9-4 gapmer format wherein 9 non-2′-O— (MOE) modified nucleotides are flanked by 4 or 5 2′-O-(MOE) modified nucleotides on one or both sides. In certain embodiments, antisense compounds targeted to a target nucleic acid comprise a steric blocking chemical modification format. In some embodiments of the steric blocking chemical modification format, every nucleotide of the antisense compound is a 2′-O-(2-methoxyethyl) (MOE) modified nucleotide. In some embodiments of the steric blocking chemical modification format, every nucleotide of the antisense compound is a tricyclo-DNA modified nucleotide. In some embodiments of the steric blocking chemical modification format, the antisense compound comprises at least one MOE modified nucleotide and at least one tricyclo-DNA modified nucleotide. Many different chemical modification patterns steric blocking antisense oligonucleotides are envisioned. For example, but in no way limiting, the steric blocking antisense oligonucleotide can comprise a mixture of different types of modifications, such as a mixture of 2′-O-(2-methoxyethyl) modifications, LNA modifications, tricyclo-DNA modifications, and DNA modifications where the DNA stretches are four nucleotides or less.

In some embodiments, an antisense compound of the present disclosure directs cleavage of a target transcript by RNase H. In such embodiments, the antisense compound can be referred to as an RNase H-dependent antisense compound. In some embodiments the antisense compound is an RNase H-dependent antisense oligonucleotide. In some embodiments, an antisense oligonucleotide of the present disclosure is an RNase H-dependent antisense oligonucleotide, and can be a single-stranded, chemically modified oligonucleotide that binds to a complementary sequence in the target transcript (e.g., a target transcript). An RNase H-dependent antisense oligonucleotide of the present disclosure reduces expression of a target gene by RNase H-mediated cleavage of the target transcript, and by inhibition of translation by steric blockade of ribosomes. In some embodiments, an antisense compound of the present disclosure is capable of mediating cleavage of at least 50%, at least 55%, at least 60%, at least 65%, at least 70%, at least 75%, at least 80%, at least 85%, at least 90%, at least 95% or more of target transcripts by RNase-H. In one embodiment, the antisense compound is capable of mediating cleavage of at least 80% of target transcripts by RNase-H. In one embodiment, the antisense compound is capable of mediating cleavage of at least 90% of target transcripts by RNase-H.

In certain embodiments, an antisense compound that targets a target transcript is from about 6 to about 24 subunits in length. In other embodiments, the antisense compound that targets a target transcript is from about 8 to about 80 subunits in length. For example, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments, the antisense compounds are less than 40 linked subunits in length. In some embodiments, the antisense compounds are from about 10 to about 30 linked subunits in length. In some embodiments, the antisense compounds are from about 12 to about 25 linked subunits in length. In some embodiments, the antisense compounds are from about 15 to about 20 linked subunits in length. In some embodiments, the antisense compound is an antisense oligonucleotide that targets a target transcript, and the linked subunits are linked nucleotides.

In certain embodiments antisense compounds targeted to a target transcript can be shortened or truncated. For example, a single subunit can be deleted from the 5′ end (5′ truncation), or alternatively from the 3′ end (3′ truncation). A shortened or truncated antisense compound targeted to a target transcript can have two subunits deleted from the 5′ end, or alternatively can have two subunits deleted from the 3′ end, of the antisense compound. Alternatively, the deleted nucleosides can be dispersed throughout the antisense compound, for example, in an antisense compound having one nucleoside deleted from the 5′ end and one nucleoside deleted from the 3′ end.

When a single additional subunit is present in a lengthened antisense compound, the additional subunit can be located at the 5′ or 3′ end of the antisense compound. When two or more additional subunits are present, the added subunits can be adjacent to each other, for example, in an antisense compound having two subunits added to the 5′ end (5′ addition), or alternatively to the 3′ end (3′ addition), of the antisense compound. Alternatively, the added subunits can be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

In certain embodiments, the antisense oligonucleotide comprises the formula:

A-B-C, wherein:
A comprises from about 0 to about 18 modified nucleotides;
B comprises from about 0 to about 4 deoxyribonucleic acid (DNA) nucleotides and/or DNA-like nucleotides; and
C comprises from about 0 to about 18 modified nucleotides;
and the overall length of the antisense oligonucleotide is about 10 to about 30 nucleotides.

Antisense oligonucleotides that contain 4 or fewer DNA and/or DNA-like nucleotides in “B” should not recruit RNase H and direct cleavage of a target. In these instances, the antisense oligonucleotide is not a gapmer format, but can rather act as a steric blocker.

### Conjugated Antisense Oligonucleotides

Antisense oligonucleotides can be covalently linked to one or more moieties, ligands, or conjugates which enhance the activity, cellular distribution or cellular uptake of the resulting antisense oligonucleotides. Antisense oligonucleotides can be covalently linked to one or more moieties, ligands, or conjugates which enhance and/or optimize pharmacokinetic parameters. Various pharmacokinetic parameters are absorbance, concentration of a compound in the body, the degree to which a compound permeates the body, the rate of elimination/clearance of a compound, the volume of plasma cleared of a compound per unit time, and others.

Conjugate groups can include hydrophobic moieties. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and Lithocholic acid (LCA). In a certain embodiment, the fatty acid selected from the group consisting of Eicosapentanoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an antisense compound of the disclosure is conjugated to a lipophilic moiety. In one embodiment, the lipophilic moiety is a ligand that includes a cationic group. In certain embodiments, the lipophilic moiety is selected from the group consisting of cholesterol, vitamin E, vitamin K, vitamin A, folic acid, or a cationic dye (e.g., Cy3). In an exemplary embodiment, the lipophilic moiety is a cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine. Diverse lipid conjugates can preferentially drive oligonucleotide uptake into different tissues (Biscans et al, Nucleic Acids Res. 2019, 47, 1082-1096).

Additional conjugate groups include carbohydrates, phospholipids, antibodies, peptides, biotin, phenazine, folate, phenanthridine, anthraquinone, acridine, fluoresceins, rhodamines, coumarins, and dyes. In some embodiments, conjugation of a ligand to an antisense oligonucleotide allows recognition by cell-surface receptors (see, e.g., Wolfrum et al., Nat. Biotechnol. 2007, 25:1149-1157; Hostetler et al., Antiviral Chem. Chemother. 2001, 12:61-70; and Prakash et al., Nucleic Acids Res. 2014, 42:8796-807). In certain embodiments, the conjugate is a fibronectin type III (FN3) domain, such as a centyrin protein (see, e.g., Goldberg et al., Protein Eng Des Sel. 2016, 29(12):563-572). The various moieties, ligands, or conjugates of the disclosure and means to conjugate them to antisense compounds are described in further detail in WO2017/030973A1 and WO2018/031933A2, incorporated herein by reference.

Antisense oligonucleotides can also be modified to have one or more stabilizing groups that are generally attached to one or both termini of antisense oligonucleotides to enhance properties such as, for example, nuclease stability. Included in stabilizing groups are cap structures. These terminal modifications protect the antisense oligonucleotide having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps. Further 3′ and 5′-stabilizing groups that can be used to cap one or both ends of an antisense oligonucleotide to impart nuclease stability include those disclosed in WO 03/004602 published on Jan. 16, 2003.

In some embodiments, an antisense oligonucleotide of the present disclosure comprises a conjugate. In one embodiment, an antisense oligonucleotide of the present disclosure comprises a antisense oligonucleotide sequence and a conjugate, wherein the conjugate is linked to the antisense oligonucleotide sequence. In some embodiments, the conjugate is selected from any of the conjugates described herein, for example, a hydrophobic conjugate, a tissue-targeting conjugate, or a conjugate designed to optimize pharmacokinetic parameters. A hydrophobic conjugate useful for conjugating to antisense oligonucleotides of the present disclosure, includes a hexadecyloxypropyl conjugate, a cholesterol conjugate, a polyunsaturated fatty acid conjugate, and others known in the art that can improve cellular uptake of a conjugate antisense oligonucleotide. In some embodiments, the conjugate can be a tissue-targeting conjugate, for example, a carbohydrate conjugate, or a peptide conjugate, or any conjugate known in the art that can target an antisense oligonucleotide of the present disclosure to a specific tissue. In some embodiments, an antisense oligonucleotide of the present disclosure is conjugated with a polyethylene glycol conjugate. In one embodiment, a polyethylene glycol conjugate antisense oligonucleotide optimizes pharmacokinetic properties of the antisense oligonucleotide.

In some embodiments, the present disclosure provides biocleavable analogues of antisense oligonucleotides described herein. In such cases, biocleavable analogues comprise a hydrophobic conjugate that leads to stronger association with cell membranes and a linker. In one embodiment, the linker is a cleavable linker that when cleaved, releases the antisense oligonucleotide, e.g., releases the antisense oligonucleotide into endosomes. In some embodiments, an antisense compound comprises a cleavable linker, wherein the cleavable linker degrades when cleaved. In some embodiments, the linker is a nuclease-cleavable linker comprising a phosphodiester linkage. In some embodiments, the nuclease-cleavable linker comprising a phosphodiester linkage is about 2 to about 8 nucleotides in length. For example, a nuclease-cleavable phosphodiester linker can be 3, 4, 5, 6, 7, 8 nucleotides in length, or longer, e.g., 9, 10, 11, 12, 13, 14, 15, 16, 17, 18 nucleotides in length, or longer. In one embodiment, the nuclease-cleavable linker comprises about 6 nucleotides. In some embodiments, the cleavable linker is cleaved after cellular internalization. In some embodiments, the cleavable linker is cleaved within an endosome. In some embodiments, the cleavable linker is cleaved under reducing conditions. In some embodiments, the cleavable linker is cleaved under changing pH conditions, for example the cleavable linker is cleaved when the pH decreases, or when the pH increases. In some embodiments, the cleavable linker is cleaved by an intracellular nuclease or protease. In some embodiments, the cleavable linker is cleaved by an endosomal nuclease or protease.

### siRNASO Linkers

Each siRNA and ASO of the siRNASO compounds of the disclosure attached to each other via a linker. In certain embodiments, the linker is a non-nucleotide linker. In certain embodiments, the non-nucleotide linker comprises an ethylene glycol chain, an alkyl chain, a peptide, an amide, a carbamate, or a combination thereof.

In certain embodiments, the alkyl chain is a C₂ to C₁₅ alkyl chain (i.e., a alkyl chain of two carbons to fifteen carbons in length). In certain embodiments, the alkyl chain is a propyl (C₃), nonyl (C₉), or dodecyl (C₁₂) chain.

In certain embodiments, the ethylene glycol chain comprises 1-15 ethylene glycol units. In certain embodiments, the ethylene glycol chain is 1 (PEG1), 2 (PEG2), 4 (PEG4), or 12 (PEG12) ethylene glycol units.

In certain embodiments, the linker is a cleavable linker. In certain embodiments, the cleavable linker comprises a phosphodiester linkage, a disulfide linkage, an acid-labile linkage, a photocleavable linkage, or a (dT)ₙ such as a dTdT dinucleotide with phosphodiester intemucleotide linkages. In certain embodiments, the acid-labile linkage comprises a β-thiopropionate linkage or a carboxydimethylmaleic anhydride (CDM) linkage.

In certain embodiments, the linker comprises a nucleotide linker. In certain embodiments, the nucleotide linker comprises (dT)n, (dA)n, (dC)n, (dG)n, or (U)n, wherein n is an integer from 2 to 30. In certain embodiments, the nucleotide linker is selected from the group consisting of: (dT)₂, (dT)₁₀, (dT)₃₀, (dA)₂, (dA)₁₀, (dC)₂, (dG)₂, (U)₂, (U)₁₀, and (dT)(dA).

In certain embodiments, the nucleotide linker further comprises a non-nucleotide linker. In certain embodiments, the non-nucleotide linker comprises an ethylene glycol chain, an alkyl chain, a peptide, an amide, a carbamate, or a combination thereof.

In certain embodiments, the alkyl chain is a C₂ to C₁₅ alkyl chain. In certain embodiments, the alkyl chain is a propyl (C₃), nonyl (C₉), or dodecyl (C₁₂) chain.

In certain embodiments, the ethylene glycol chain comprises 1-15 ethylene glycol units. In certain embodiments, the ethylene glycol chain is 1 (PEG1), 2 (PEG2), 4 (PEG4), or 12 (PEG12) ethylene glycol units.

### Exemplary siRNASO Configurations

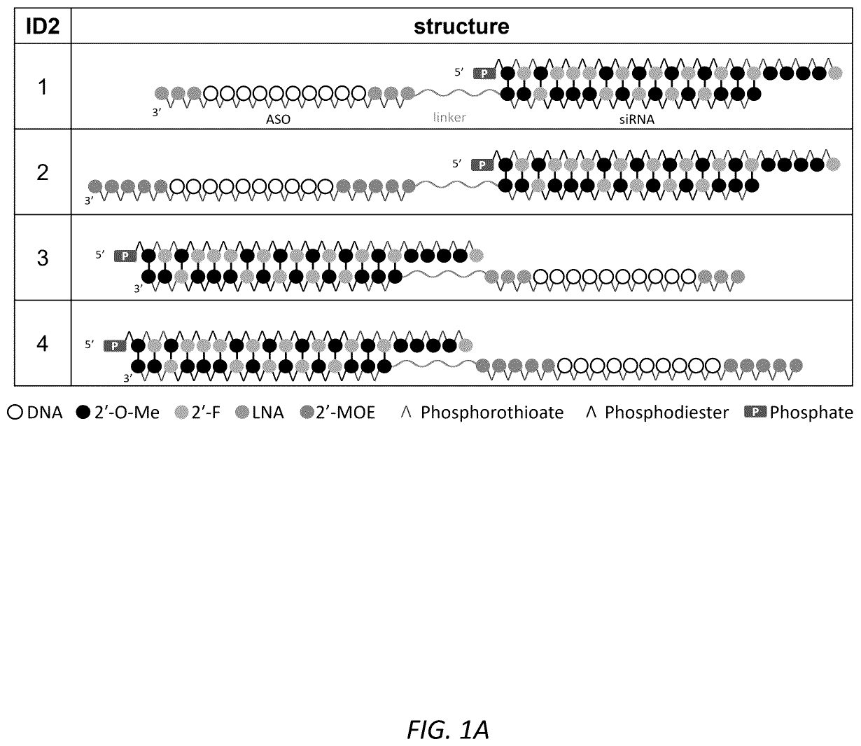

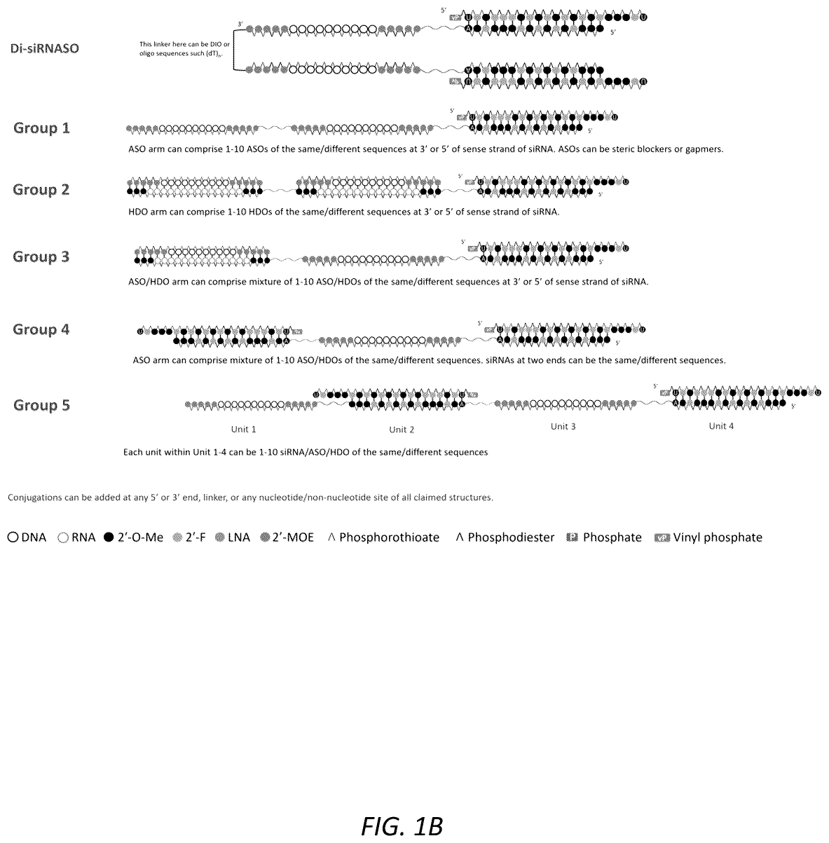

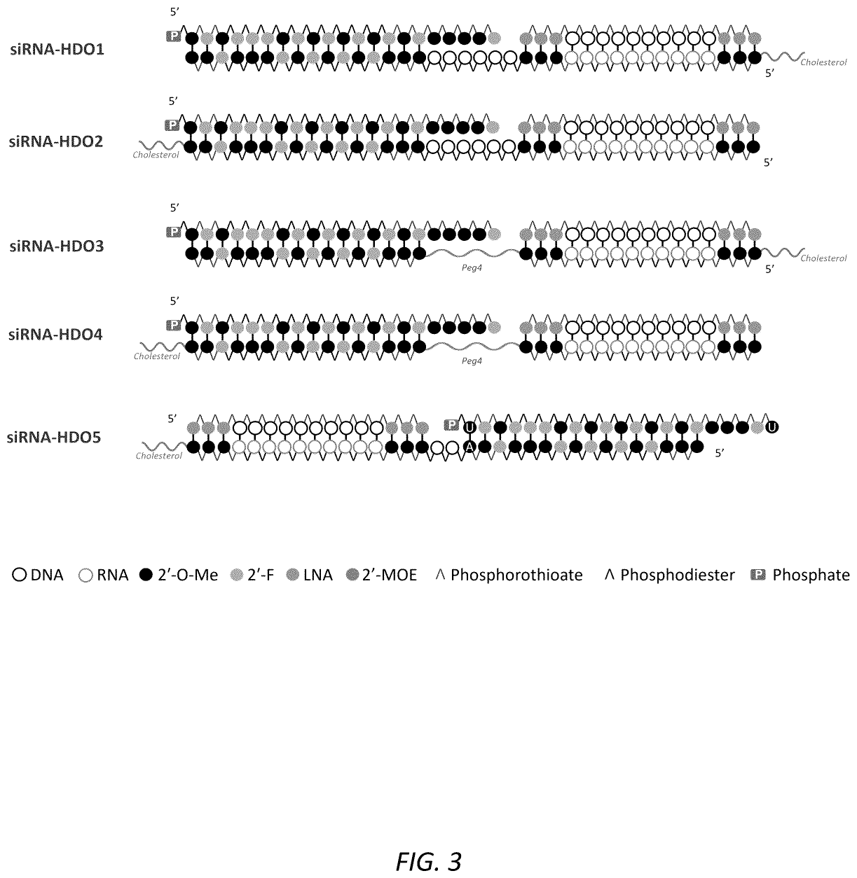

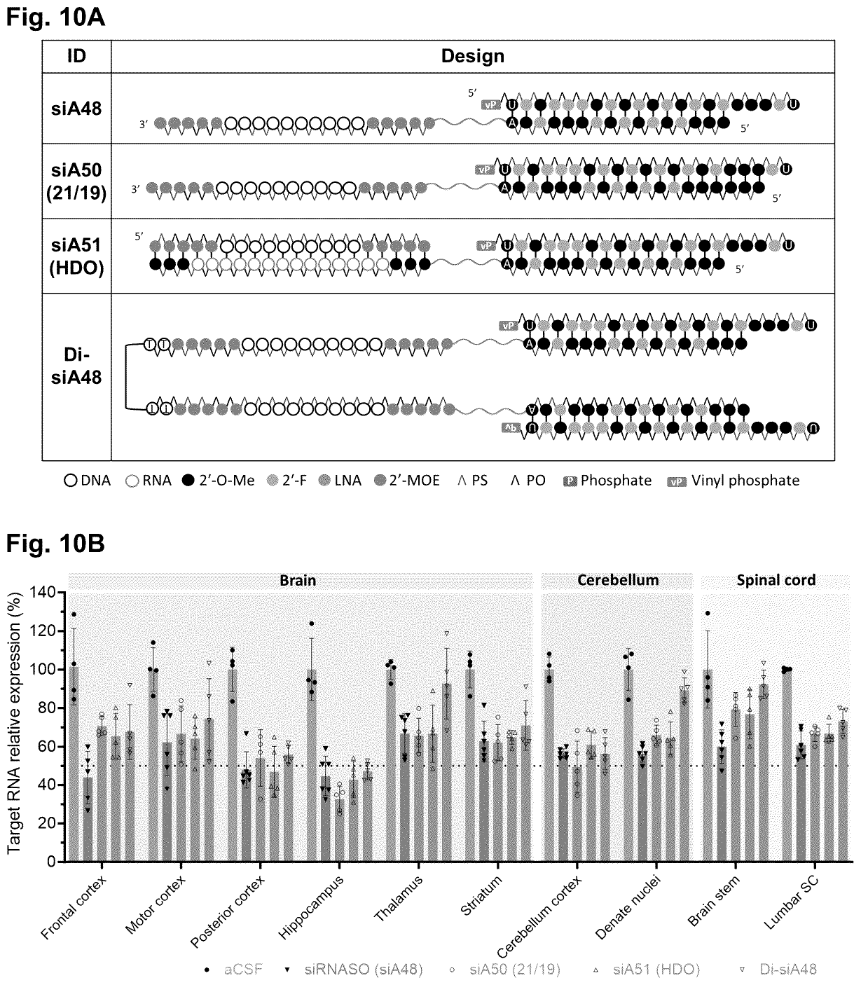

The siRNASO compounds of the disclosure may be in numerous possible configurations, with one or more siRNA attached to one or more ASO or HDO. Exemplary siRNASO configurations are shown graphically in FIG. TA, FIG. 1B, FIG. 3, and FIG. 10.

In certain embodiments, the siRNASO comprises or consisting of a first ASO, a first linker, a second ASO, a second linker, and a first siRNA comprising a first antisense strand and a first sense strand, wherein the 5′ end of the first ASO is linked to the 3′ end of the second ASO via the first linker and the 5′ end of the second ASO is linked to the 3′ end of the first sense strand via the second linker.

In certain embodiments, the siRNASO comprises or consisting of a first siRNA comprising a first antisense strand and a first sense strand, a first linker, a first ASO, a second linker, and a second siRNA comprising a second antisense strand and a second sense strand, wherein the 5′ end of the first sense strand is linked to the 3′ end of the first ASO via the first linker and the 5′ end of the first ASO is linked to the 3′ end of the second sense strand via the second linker.

In certain embodiments, the siRNASO comprises or consisting of a first ASO, a first linker, a first siRNA comprising a first antisense strand and a first sense strand, a second linker, a second ASO, a third linker, and a second siRNA comprising a second antisense strand and a second sense strand, wherein the 5′ end of the first ASO is linked to the 3′ end of the first sense strand via the first linker, the 5′ end of the first sense strand is linked to the 3′ end of the second ASO via the second linker, and the 5′ end of the second ASO is linked to the 3′ end of the second sense strand via the third linker.

In certain embodiments, the siRNASO comprises or consisting of a first siRNA comprising a first antisense strand and a first sense strand, a first linker, a first ASO, a second linker, a second ASO, a third linker, and a second siRNA comprising a second antisense strand and a second sense strand, wherein the 3′ end of the first sense strand is linked to the 5′ end of the first ASO via the first linker, the 3′ end of the first ASO is linked to the 3′ end of the second ASO via the second linker, and the 5′ end of the second ASO is linked to the 3′ end of the second sense strand via the third linker.

In certain embodiments, the first siRNA and the second siRNA have identical nucleotide sequences.

In certain embodiments, the first siRNA and the second siRNA have complementarity to the same target gene.

In certain embodiments, the first siRNA and the second siRNA have different nucleotide sequences.

In certain embodiments, the first siRNA and the second siRNA have complementarity to the different target genes.

In certain embodiments, the first ASO and the second ASO have identical nucleotide sequences.

In certain embodiments, the first ASO and the second ASO have complementarity to the same target gene.

In certain embodiments, the first ASO and the second ASO have different nucleotide sequences.

In certain embodiments, the first ASO and the second ASO have complementarity to the different target genes.

In certain embodiments, the first linker, the second linker, and the third linker are identical.

In certain embodiments, the first linker, the second linker, and the third linker are different.

### IV. Methods of Introducing siRNASO

The siRNASO compounds of the disclosure may be directly introduced into the cell (e.g., a lung cell, liver cell, or neural cell) (i.e., intracellularly); or introduced extracellularly into a cavity, interstitial space, into the circulation of an organism, introduced orally, or may be introduced by bathing a cell or organism in a solution containing the siRNASO. Vascular or extravascular circulation, the blood or lymph system, and the cerebrospinal fluid are sites where the nucleic acid may be introduced.

The siRNASO of the disclosure can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The siRNASO may be introduced along with other components that perform one or more of the following activities: enhance nucleic acid uptake by the cell or other-wise increase inhibition of the target gene.

Depending on the particular target gene and the dose of siRNASO material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, Enzyme Linked ImmunoSorbent Assay (ELISA), Western blotting, RadioImmunoAssay (RIA), other immunoassays, and Fluorescence Activated Cell Sorting (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present disclosure. Lower doses of injected material and longer times after administration of an siRNASO may result in inhibition in a smaller fraction of cells (e.g., at least 10%, 20%, 50%, 75%, 90%, or 95% of targeted cells). Quantization of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

In an exemplary aspect, the efficacy of an siRNASO of the disclosure is tested for its ability to specifically degrade target mRNA (e.g., target mRNA and/or the production of target protein) in cells. Suitable cell-based validation assays are other readily transfectable cells, for example, HeLa cells or COS cells. Cells are transfected with human cDNAs (e.g., human target cDNA). The siRNASO are co-transfected. Selective reduction in target mRNA (e.g., target mRNA) and/or target protein (e.g., target protein) is measured. Reduction of target mRNA or protein can be compared to levels of target mRNA or protein in the absence of an siRNASO or in the presence of an siRNASO that does not target mRNA. Exogenously-introduced mRNA or protein (or endogenous mRNA or protein) can be assayed for comparison purposes.

### V. Methods of Treatment

In one aspect, the present disclosure provides for both prophylactic and therapeutic methods of treating a subject at risk of (or susceptible to) developing diseases associated with the lung.

“Treatment,” or “treating,” as used herein, is defined as the application or administration of a therapeutic agent (e.g., a siRNASO) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosure provides a method for preventing in a subject, a disease or disorder as described above, by administering to the subject a therapeutic agent (e.g., an siRNASO). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

### VI. Pharmaceutical Compositions and Methods of Administration

The disclosure pertains to uses of the above-described agents for prophylactic and/or therapeutic treatments as described infra. Accordingly, the modulators (e.g., siRNASO) of the present disclosure can be incorporated into pharmaceutical compositions suitable for administration. Such compositions typically comprise the nucleic acid molecule or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, and subcutaneous, or intratracheal.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following example, which is included for purposes of illustration only and is not intended to be limiting.

### EXAMPLES

### Example 1. siRNASO Compounds

Multiple types of oligonucleotide compounds have been described, each oligonucleotide eliciting a unique mechanism of action for the inhibition of a target gene. To leverage the activity of small interfering RNAs (siRNAs) and antisense oligonucleotides (ASOs), an oligonucleotide compound was devised, attaching the ASO to the siRNA, which is herein referred to as an “siRNASO”. Numerous versions of this siRNASO were generated, schematics of which are shown in FIGS. 1A-B.

Each siRNA used in the siRNASO comprised a 21-nucleotide antisense strand and a 16-nucleotide sense strand, with a 5-nucleotide overhang region. Each ribose sugar in the siRNA was chemically modified with either 2′-OMe or 2′-F, yielding a fully chemically modified siRNA. The antisense strand was complementary to the Htt mRNA, which should lead to silencing of Htt expression. The ASO was attached to either the 5′ end or 3′ end of the sense strand

Two different ASOs were used in the siRNASO. One ASO comprised three 5′ and 3′ end LNA modifications with ten DNA nucleotides in between. The other ASO comprised five 5′ and 3′ end 2′-MOE modifications with ten DNA nucleotides in between. The ASOs were complementary to Malat1 mRNA and should lead to silencing of Malat1 expression.

The various siRNASOs were tranfected into Hepa1-6 cells at 12 nM. Table 1 below describes the various siRNASOs employed, including the linker attaching the ASO to the siRNA. The structure ID corresponds to the ID number found in FIGS. 1A-B.

TABLE 1
siRNASO IDs with linkers used.

| siRNASO ID | Structure ID | Linker |
|---|---|---|
| siRNASO A1 | 1 | PEG4 |
| siRNASO A2 | 3 | PEG4 |
| siRNASO A3 | 1 | PEG4-(dT)₂ |
| siRNASO A4 | 3 | (dT)₂-PEG4 |
| siRNASO A5 | 3 | (dT)₂ |
| siRNASO A6 | 1 | (dT)₂ |
| siRNASO A7 | 1 | (dT)₁₀ |
| siRNASO A8 | 1 | (dT)₃₀ |
| siRNASO A9 | 1 | (dA)₂ |
| siRNASO A10 | 1 | (dA)₁₀ |
| siRNASO A11 | 1 | (rU)₂ |
| siRNASO A12 | 1 | (rU)₁₀ |
| siRNASO A13 | 1 | (dT)(dA) |
| siRNASO A14 | 1 | (dG)₂ |
| siRNASO A15 | 1 | (dC)₂ |
| siRNASO A16 | 2 | (dT)₂ |
| siRNASO A17 | 4 | (dT)₂ |

In the above recited table, “PEG4” represents tetraethylene glycol; “d” represents a DNA nucleotide; “r” represents an RNA nucleotide; “T” represents thymine; “A” represents adenine; “G” represents guanine; “C” represents cytosine; “U” represents uracil; and the subscript integer (e.g., 2, 10, or 30) represents the number of repeated nucleotides (i.e., (dT)₂ represents (dT)(dT) and (dT)₁₀ represents (dT)(dT)(dT)(dT)(dT)(dT)(dT)(dT)(dT)(dT)).

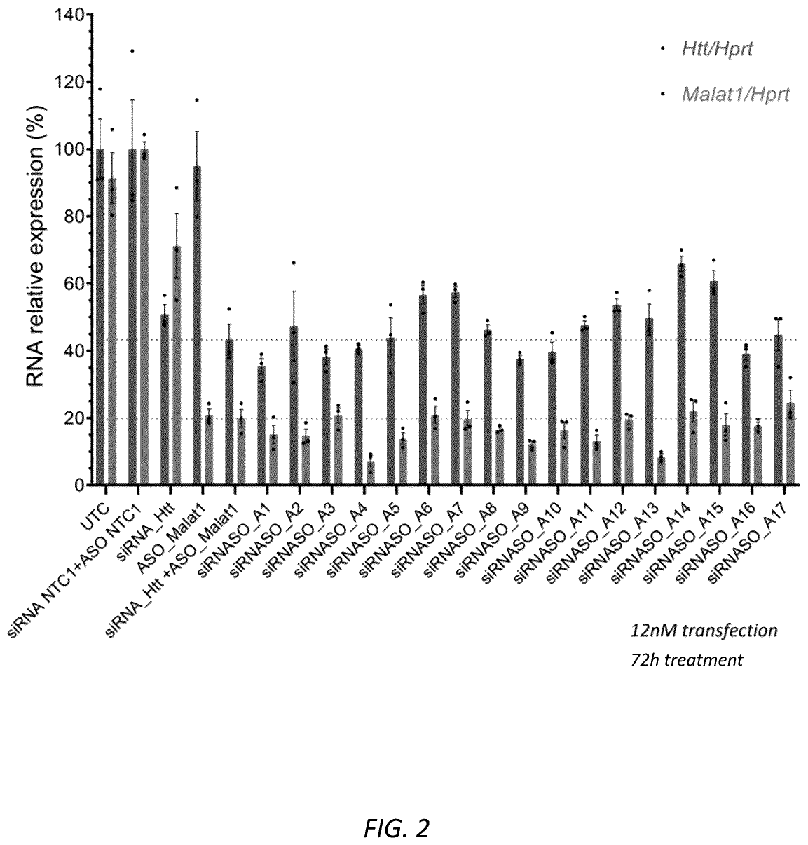

As shown in FIG. 2, each of the tested siRNASO formats tested were capable of robustly silencing both Hit (through the action of the siRNA component) and Malat1 (through the action of the ASO component). The level of silencing achieved was as good or better than an Hit-targeting siRNA alone or a Malat1-targeting ASO alone. Table 2 below describes the various siRNAs, ASOs, and siRNASOs employed.

TABLE 2
siRNAs, ASOs, and siRNASO.

| Oligo ID | Sequence |
|---|---|
| siRNA NTC1_as | P(mU)#(fA)#(mA)(fU)(fC)(fG)(mU)(fA)(mU)(fU)(mU)(fG)(mU)(fC)#(mA) |
| | #(fA)#(mU)#(mC)#(mA)#(mU)#(fU) |
| siRNA NTC1_s | (mU)#(mU)#(mG)(fA)(mC)(fA)(mA)(fA)(mU)(fA)(mC)(mG)(mA)(fU)#(mU) |
| | #(mA) |
| ASO NTC1 | (1G)#(1A)#(1G)#(dT)#(d5C)#(dG)#(dT)#(dT)#(dA)#(d5C)#(d5C)#(dT)#(dT)# |
| | (1A)#(1C)#(1A) |
| siRNA_Htt_as | P(mU)#(fU)#(mA)(fA)(fU)(fC)(mU)(fC)(mU)(fU)(mU)(fA)(mC)(fU)#(mG)# |
| | (fA)#(mU)#(mA)#(mU)#(mA)#(fU) |
| siRNA_Htt_s | (mU)#(mC)#(mA)(fG)(mU)(fA)(mA)(fA)(mG)(fA)(mG)(mA)(mU)(fU)#(mA) |
| | #(mA) |
| ASO_Malat1 | (1C)#(1T)#(1A)#(dG)#(dT)#(dT)#(d5C)#(dA)#(d5C)#(dT)#(dG)#(dA)#(dA)# |
| | (1T)#(1G)#(1C) |
| siRNASO_A1_s | (mU)#(mC)#(mA)(fG)(mU)(fA)(mA)(fA)(mG)(fA)(mG)(mA)(mU)(fU)#(mA) |
| | #(mA)(PEG4)(1C)#(1T)#(1A)#(dG)#(dT)#(dT)#(d5C)#(dA)#(d5C)#(dT)#(dG) |
| | #(dA)#(dA)#(1T)#(1G)#(IC) |
| siRNASO_A2_s | (1C)#(1T)#(1A)#(dG)#(dT)#(dT)#(d5C)#(dA)#(d5C)#(dT)#(dG)#(dA)#(dA)# |
| | (1T)#(1G)#(1C)(PEG4)(mU)#(mC)#(mA)(fG)(mU)(fA)(mA)(fA)(mG)(fA)(mG) |
| | (mA)(mU)(fU)#(mA)#(mA) |
| siRNASO_A3_s | (mU)#(mC)#(mA)(fG)(mU)(fA)(mA)(fA)(mG)(fA)(mG)(mA)(mU)(fU)#(mA) |
| | #(mA)(PEG4)(dT)(dT)(1C)#(1T)#(1A)#(dG)#(dT)#(dT)#(d5C)#(dA)#(d5C) |
| | #(dT)#(dG)#(dA)#(dA)#(1T)#(1G)#(1C) |
| siRNASO_A4_s | (1C)#(1T)#(1A)#(dG)#(dT)#(dT)#(d5C)#(dA)#(d5C)#(dT)#(dG)#(dA)#(dA)# |
| | (1T)#(1G)#(1C)(dT)(dT)(PEG4)(mU)#(mC)#(mA)(fG)(mU)(fA)(mA)(fA)(mG) |
| | (fA)(mG)(mA)(mU)(fU)#(mA)#(mA) |
| siRNASO_A5_s | (1C)#(1T)#(1A)#(dG)#(dT)#(dT)#(d5C)#(dA)#(d5C)#(dT)#(dG)#(dA)#(dA)# |
| | (1T)#(1G)#(1C)(dT)(dT)(mU)#(mC)#(mA)(fG)(mU)(fA)(mA)(fA)(mG)(fA) |
| | (mG)(mA)(mU)(fU)#(mA)#(mA) |
| siRNASO_A6_s | (mU)#(mC)#(mA)(fG)(mU)(fA)(mA)(fA)(mG)(fA)(mG)(mA)(mU)(fU)#(mA) |
| | #(mA)(dT)(dT)(1C)#(1T)#(1A)#(dG)#(dT)#(dT)#(d5C)#(dA)#(d5C)#(dT)# |
| | (dG)#(dA)#(dA)#(IT)#(1G)#(IC) |
| siRNASO_A7_s | (mU)#(mC)#(mA)(fG)(mU)(fA)(mA)(fA)(mG)(fA)(mG)(mA)(mU)(fU)#(mA) |
| | #(mA)(dT)(dT)(dT)(dT)(dT)(dT)(dT)(dT)(dT)(dT)(1C)#(1T)#(1A)#(dG)# |
| | (dT)#(dT)#(d5C)#(dA)#(d5C)#(dT)#(dG)#(dA)#(dA)#(1T)#(1G)#(1C) |
| siRNASO_A8_s | (mU)#(mC)#(mA)(fG)(mU)(fA)(mA)(fA)(mG)(fA)(mG)(mA)(mU)(fU)#(mA) |
| | #(mA)(dT)(dT)(dT)(dT)(dT)(dT)(dT)(dT)(dT)(dT)(dT)(dT)(dT)(dT)(dT) |
| | (dT)(dT)(dT)(dT)(dT)(dT)(dT)(dT)(dT)(dT)(dT)(dT)(dT)(dT)(dT)(1C)#(1T) |
| | #(1A)#(dG)#(dT)#(dT)#(d5C)#(dA)#(d5C)#(dT)#(dG)#(dA)#(dA)#(1T)#(1G)#(1C) |
| siRNASO_A9_s | (mU)#(mC)#(mA)(fG)(mU)(fA)(mA)(fA)(mG)(fA)(mG)(mA)(mU)(fU)#(mA)# |
| | (mA)(dA)(dA)(1C)#(1T)#(1A)#(dG)#(dT)#(dT)#(d5C)#(dA)#(d5C)#(dT)# |
| | (dG)#(dA)#(dA)#(1T)#(1G)#(1C) |
| siRNASO_A10_s | (mU)#(mC)#(mA)(fG)(mU)(fA)(mA)(fA)(mG)(fA)(mG)(mA)(mU)(fU)#(mA)# |
| | (mA)(dA)(dA)(dA)(dA)(dA)(dA)(dA)(dA)(dA)(dA)(1C)#(1T)#(1A)#(dG)# |
| | (dT)#(dT)#(d5C)#(dA)#(d5C)#(dT)#(dG)#(dA)#(dA)#(1T)#(1G)#(1C) |
| siRNASO_A11_s | (mU)#(mC)#(mA)(fG)(mU)(fA)(mA)(fA)(mG)(fA)(mG)(mA)(mU)(fU)# |
| | (mA)#(mA)(rU)(rU)(1C)#(1T)#(1A)#(dG)#(dT)#(dT)#(d5C)#(dA)#(d5C)# |
| | (dT)#(dG)#(dA)#(dA)#(1T)#(1G)#(1C) |
| siRNASO_A12_s | (mU)#(mC)#(mA)(fG)(mU)(fA)(mA)(fA)(mG)(fA)(mG)(mA)(mU)(fU)# |
| | (mA)#(mA)(rU)(rU)(rU)(rU)(rU)(rU)(rU)(rU)(rU)(rU)(1C)#(1T)#(1A)#(dG) |
| | #(dT)#(dT)#(d5C)#(dA)#(d5C)#(dT)#(dG)#(dA)#(dA)#(1T)#(1G)#(1C) |
| siRNASO_A13_s | (mU)#(mC)#(mA)(fG)(mU)(fA)(mA)(fA)(mG)(fA)(mG)(mA)(mU)(fU)#(mA) |
| | #(mA)(dT)(dA)(1C)#(1T)#(1A)#(dG)#(dT)#(dT)#(d5C)#(dA)#(d5C)#(dT)# |
| | (dG)#(dA)#(dA)#(1T)#(1G)#(1C) |
| siRNASO_A14_s | (mU)#(mC)#(mA)(fG)(mU)(fA)(mA)(fA)(mG)(fA)(mG)(mA)(mU)(fU)#(mA) |
| | #(mA)(dG)(dG)(IC)#(IT)#(IA)#(dG)#(dT)#(dT)#(d5C)#(dA)#(d5C)#(dT)# |
| | (dG)#(dA)#(dA)#(IT)#(1G)#(IC) |
| siRNASO_A15_s | (mU)#(mC)#(mA)(fG)(mU)(fA)(mA)(fA)(mG)(fA)(mG)(mA)(mU)(fU)#(mA) |
| | #(mA)(d5C)(d5C)(IC)#(IT)#(1A)#(dG)#(dT)#(dT)#(d5C)#(dA)#(d5C)# |
| | (dT)#(dG)#(dA)#(dA)#(IT)#(1G)#(IC) |
| siRNASO_A16_s | (mU)#(mC)#(mA)(fG)(mU)(fA)(mA)(fA)(mG)(fA)(mG)(mA)(mU)(fU)#(mA) |
| | #(mA)(dT)(dT)(eG)#(eG)#(eG)#(eU)#(eC)#(dA)#(dG)#(d5C)#(dT)#(dG) |
| | #(d5C)#(d5C)#(dA)#(dA)#(dT)#(eG)#(eC)#(eU)#(eA)#(eG) |
| siRNASO_A17_s | (eG)#(eG)#(eG)#(eU)#(eC)#(dA)#(dG)#(d5C)#(dT)#(dG)#(d5C)#(d5C)# |
| | (dA)#(dA)#(dT)#(eG)#(eC)#(eU)#(eA)#(eG)(dT)(dT)(mU)#(mC)#(mA)(fG) |
| | (mU)(fA)(mA)(fA)(mG)(fA)(mG)(mA)(mU)(fU)#(mA)#(mA) |

Table 3 below shows the legend for the sequences presented in Tables 2 and 4-8.

TABLE 3
Legend.

| | Nomenclature |
|---|---|
| Standard 2′- | |
| deoxyribonucleosides | |
| dA | (dA) |
| dC | (dC) |
| 5-Me-dC | (d5C) |
| dG | (dG) |
| dT | (dT) |
| Standard ribonucleosides | |
| rA | (rA) |
| rC | (rC) |
| rG | (rG) |
| rU | (rU) |
| 2′-modified nucleosides | |
| 2′-OMe A | (mA) |
| 2′-OMe C | (mC) |
| 2′-OMe G | (mG) |
| 2′-OMe U | (mU) |
| 2′-Fluoro A | (fA) |
| 2′-Fluoro C | (fC) |
| 2′-Fluoro G | (fG) |
| 2′-Fluoro U | (fU) |
| 2-4′-LNA A | (1A) |
| 2-4′-LNA C | (IC) |
| 2-4′-LNA G | (1G) |
| 2-4′-LNA T | (IT) |
| 2′-MOE A | (eA) |
| 2′-MOE C | (eC) |
| 2′-MOE G | (eG) |
| 2′-MOE U | (eU) |
| linkages | |
| Phosphorothioate bond | )#( |
| Phosphodiester bond | )( |
| 5′ and 3′ end modification | |
| 5′-phosphate | P |
| 5′-Teg-Cholesterol | TegChol |
| 5′-vinyl phosphate | V |
| 3′-Teg-cholesterol | TegChol |
| New GalNAc | GalNac |

The siRNASOs described above employed a single-stranded ASO component. The double stranded heteroduplex oligonucleotide (HDO), a variation of an ASO, was next tested in the siRNASO format. As shown in FIG. 3, five different HDO-containing siRNASOs were tested. Each format employed the same Htt-targeting siRNA with the same chemical modification pattern as of that shown in FIGS. 1A-B. In each format, the HDO component comprised a first strand linked to either the 5′ or 3′ end of the siRNA sense strand. This first strand comprised, from 5′ to 3′, three 2′-OMe modified nucleotides, ten unmodified RNA nucleotides, and three 2′-OMe modified nucleotides. Annealed to the first strand was a complementary second strand comprising a gapmer ASO. The second strand comprised, from 5′ to 3′, three LNA modified nucleotides, ten unmodified DNA nucleotides, and three LNA modified nucleotides.

The various HDO-based siRNASOs were incubated with Hepa1-6 cells at 2 μM. Table 4 below describes the various HDO-based siRNASOs employed, including the linker attaching the HDO to the siRNA.

TABLE 4
siRNASO IDs with linkers used.

| SiRNASO ID | Structure | Linker |
|---|---|---|
| siRNA-HDO1 | Cholesterol_5′-HDO-siRNA-3′ | (dT)₆ |
| siRNA-HDO2 | 5′-HDO-siRNA-3′_Cholesterol | (dT)₆ |
| siRNA-HDO3 | Cholesterol_5′-HDO-siRNA-3′ | PEG4 |
| siRNA-HDO4 | 5′-HDO-siRNA-3′_Cholesterol | PEG4 |
| siRNA-HDO5 | 5′-siRNA-HDO-3′_Cholesterol | (dT)₂ |

In the above recited table, “PEG4” represents tetraethylene glycol; “d” represents a DNA nucleotide; “T” represents thymine; the subscript integer (e.g., 2 or 6) represents the number of repeated nucleotides (i.e., (dT)₂ represents (dT)(dT) and (dT)₆ represents (dT)(dT)(dH)(dT)(dT)(dT)).

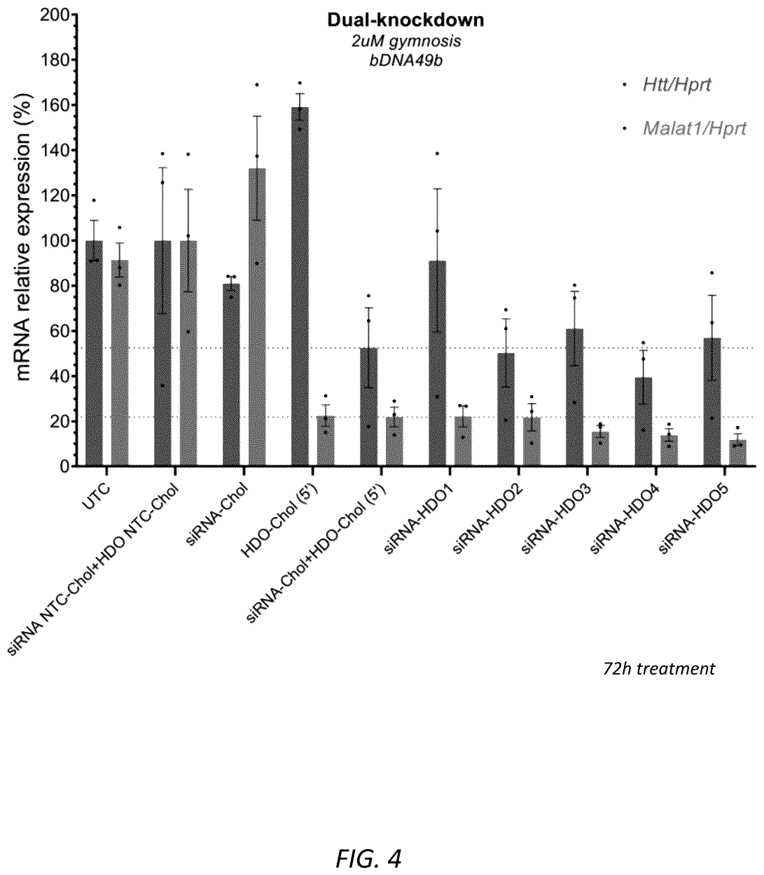

As shown in FIG. 4, each of the tested HDO-based siRNASO formats tested were capable of robustly silencing both Htt (through the action of the siRNA component) and Malat1 (through the action of the HDO component). The level of silencing achieved was as good or better than an Hit-targeting siRNA alone or a Mal at I-targeting HDO alone. Table 5 below describes the various siRNAs and siRNASOs employed.

TABLE 5
siRNAs and siRNASOs.

| Oligo ID | Sequence |
|---|---|
| siRNA NTC_Chol s | (mU)#(mU)#(mG)(fA)(mC)(fA)(mA)(fA)(mU)(fA)(mC)(mG)(mA)(fU) |
| | #(mU)#(mA)-TegChol |
| siRNA NTC_Chol_as | (mU)#(mU)#(mG)(fA)(mC)(fA)(mA)(fA)(mU)(fA)(mC)(mG)(mA)(fU) |
| | #(mU)#(mA) |
| Chol_NTC_HDO_s | TegChol-(mU)#(mG)#(mU)#(rA)(rA)(rG)(rG)(rU)(rA)(rA)(rC) |
| | (rG)(rA)#(mC)#(mU)#(mC) |
| NTC_LNA_gapmer | (1G)#(1A)#(1G)#(dT)#(d5C)#(dG)#(dT)#(dT)#(dA)#(d5C)#(d5C)# |
| | (dT)#(dT)#(1A)#(1C)#(1A) |
| Htt_siRNA_s_Chol | (mU)#(mC)#(mA)(fG)(mU)(fA)(mA)(fA)(mG)(fA)(mG)(mA)(mU)(fU) |
| | #(mA)#(mA)-TegChol |
| siRNA_Htt_as | P(mU)#(fU)#(mA)(fA)(fU)(fC)(mU)(fC)(mU)(fU)(mU)(fA)(mC) |
| | (fU)#(mG)#(fA)#(mU)#(mA)#(mU)#(mA)#(fU) |
| siRNASO_HDO1_s | TegChol-(mG)#(mC)#(mA)#(rU)(rU)(rC)(rA)(rG)(rU)(rG)(rA) |
| | (rA)(rC)#(mU)#(mA)#(mG)(dT)(dT)(dT)(dT)(dT)(dT)(mU)#(mC) |
| | #(mA)(fG)(mU)(fA)(mA)(fA)(mG)(fA)(mG)(mA)(mU)(fU)#(mA)#(mA) |
| siRNASO_HDO2_s | (mG)#(mC)#(mA)#(rU)(rU)(rC)(rA)(rG)(rU)(rG)(rA)(rA)(rC)#(mU) |
| | #(mA)#(mG)(dT)(dT)(dT)(dT)(dT)(dT)(mU)#(mC)#(mA)(fG)(mU) |
| | (fA)(mA)(fA)(mG)(fA)(mG)(mA)(mU)(fU)#(mA)#(mA)-TegChol |
| siRNASO_HDO3_s | TegChol-(mG)#(mC)#(mA)#(rU)(rU)(rC)(rA)(rG)(rU)(rG)(rA)(rA) |
| | (rC)#(mU)#(mA)#(mG)(PEG4)(mU)#(mC)#(mA)(fG)(mU)(fA)(mA)(fA) |
| | (mG)(fA)(mG)(mA)(mU)(fU)#(mA)#(mA) |
| siRNASO_HDO4_s | (mG)#(mC)#(mA)#(rU)(rU)(rC)(rA)(rG)(rU)(rG)(rA)(rA)(rC)# |
| | (mU)#(mA)#(mG)(PEG4)(mU)#(mC)#(mA)(fG)(mU)(fA)(mA)(fA)(mG) |
| | (fA)(mG)(mA)(mU)(fU)#(mA)#(mA)-TegChol |
| siRNASO_HDO5_s | (mU)#(mC)#(mA)(fG)(mU)(fA)(mA)(fA)(mG)(fA)(mG)(mA)(mU)(fU) |
| | #(mA)#(mA)(dT)(dT)(mG)#(mC)#(mA)#(rU)(rU)(rC)(rA)(rG)(rU) |
| | (rG)(rA)(rA)(rC)#(mU)#(mA)#(mG)-TegChol |
| Malat1_HDO_s_Chol | (mG)#(mC)#(mA)#(rU)(rU)(rC)(rA)(rG)(rU)(rG)(rA)(rA)(rC)#(mU) |
| | #(mA)#(mG)-TegChol |
| Malat1_HDO_as | (IC)#(IT)#(1A)#(dG)#(dT)#(dT)#(d5C)#(dA)#(d5C)#(dT)#(dG)# |
| | (dA)#(dA)#(IT)#(1G)#(IC) |

### Example 2. In Vivo Use of siRNASO Compounds

SiRNASO was next tested in vivo in a mouse model for different gene targets:

### Adam 33 Gene

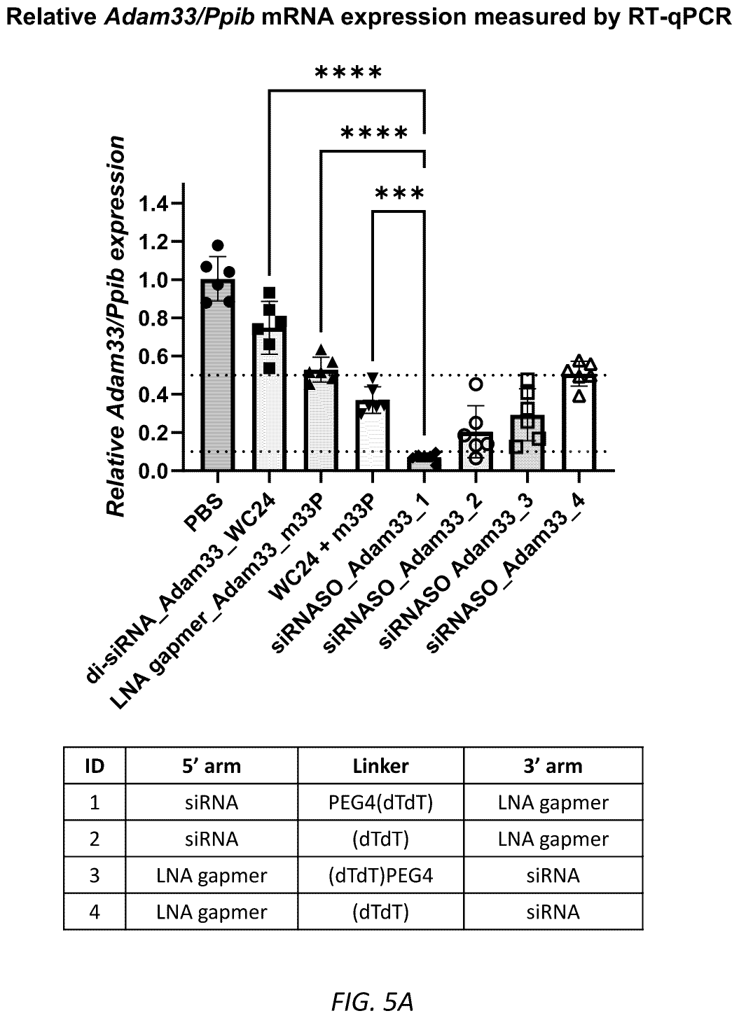

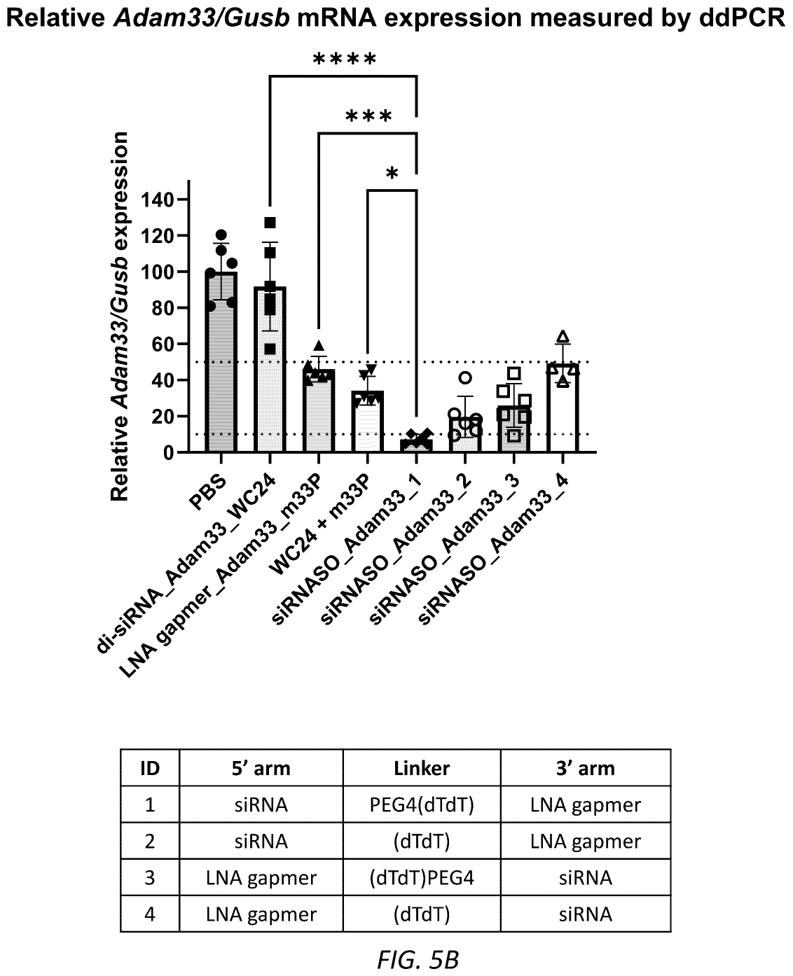

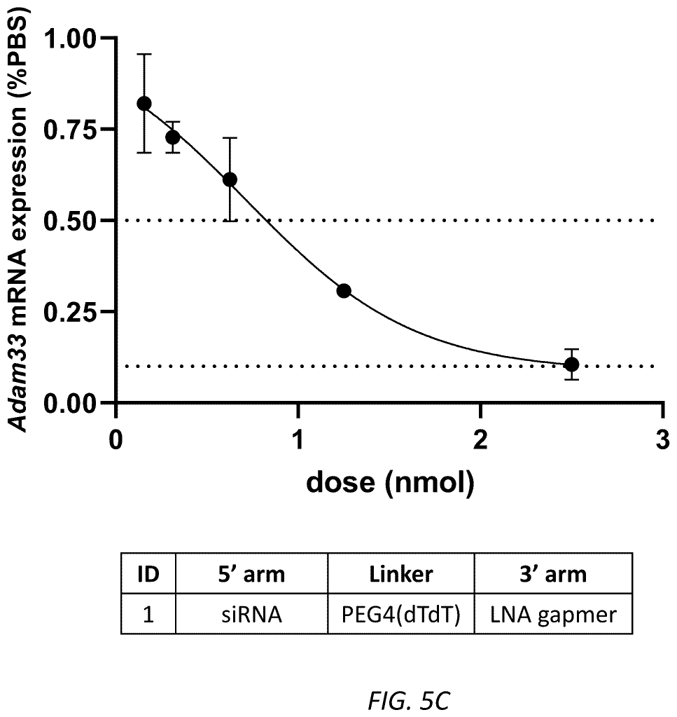

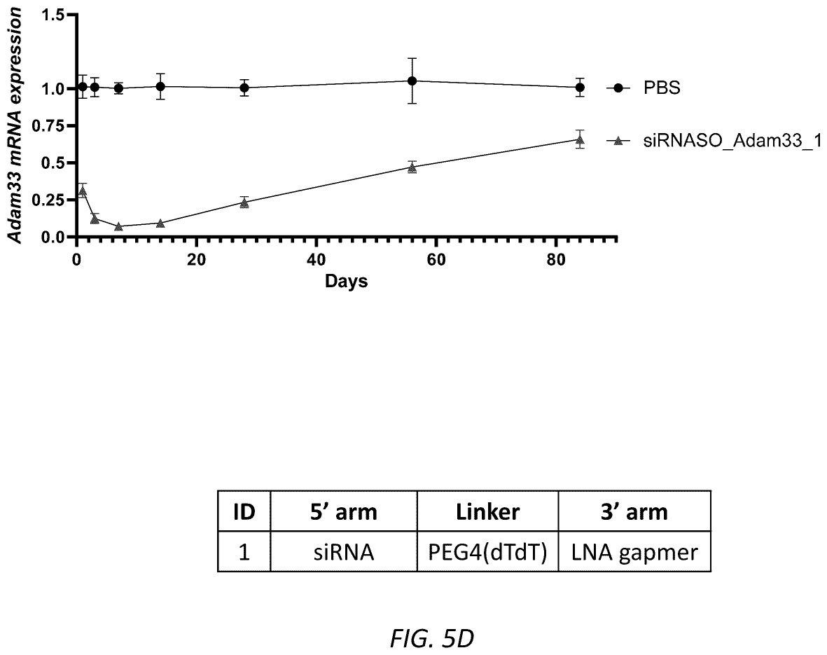

One of several siRNASO formats were administered intratracheally to mice at 2.5 nmol, where both the siRNA component and the ASO component targeted the gene Adam33. The siRNASOs were compared against a di-branched siRNA (i.e., two siRNA targeting Adam33 linked together), an LNA gapmer alone, and a mixture of the di-branched siRNA and the LNA gapmer (unlinked to each other). In each instance, the siRNASO achieved substantially higher silencing of Adam33 in the lung of the mouse (FIG. 4). When both siRNA and ASO arms target the same gene (Adam33), the siRNASO (the 5^{th} bar from the left) can achieve more robust silencing than the co-injection of di-siRNA and ASO (the 4^{th} bar from the left)(FIGS. 5A-D).

Table 6 below describes the various siRNAs, gapmers, and siRNASOs employed.

TABLE 6
siRNAs, gapmers, and siRNASOs.

| Oligo ID | Sequence |
|---|---|
| di-siRNA Adam33 WC24 s | (mU)#(mC)#(mA)(fG)(mC)(fA)(mG)(fC)(mA)(fA)(mA)(mG)(mU)(fC)#(mA) |
| | #(mA)-DIO |
| di-siRNA_Adam33_WC24 as | V(mU)#(fU)#(mG)(fA)(fC)(fU)(mU)(fU)(mG)(fC)(mU)(fG)(mC)(fU)#(mG) |
| | #(fA)#(mG)#(mC)#(mA)#(fC)#(mU) |
| LNA gapmer_Adam33 m33P | (1T)#(1A)#(1A)#(dG)#(d5C)#(dT)#(d5C)#(dA)#(dG)#(dA)#(dG)#(dT)#(1T) |
| | #(1C)#(1G) |
| siRNASO_Adam33_1_s | (mU)#(mC)#(mA)(fG)(mC)(fA)(mG)(fC)(mA)(fA)(mA)(mG)(mU)(fC)#(mA) |
| | #(mA)(PEG4)(dT)(dT)(1T)#(1A)#(1A)#(dG)#(d5C)#(dT)#(d5C)#(dA)#(dG) |
| | #(dA)#(dG)#(dT)#(1T)#(1C)#(1G) |
| siRNASO_Adam33_2_s | (mU)#(mC)#(mA)(fG)(mC)(fA)(mG)(fC)(mA)(fA)(mA)(mG)(mU)(fC)#(mA) |
| | #(mA)(dT)(dT)(1T)#(1A)#(1A)#(dG)#(d5C)#(dT)#(d5C)#(dA)#(dG)#(dA) |
| | #(dG)#(dT)#(1T)#(1C)#(1G) |
| siRNASO_Adam33_3_s | (1T)#(1A)#(1A)#(dG)#(d5C)#(dT)#(d5C)#(dA)#(dG)#(dA)#(dG)#(dT)#(1T) |
| | #(1C)#(1G)(dT)(dT)(PEG4)(mU)#(mC)#(mA)(fG)(mC)(fA)(mG)(fC)(mA)(fA) |
| | (mA)(mG)(mU)(fC)#(mA)#(mA) |
| siRNASO_Adam33_4_s | (1T)#(1A)#(1A)#(dG)#(d5C)#(dT)#(d5C)#(dA)#(dG)#(dA)#(dG)#(dT)#(1T) |
| | #(1C)#(1G)(dT)(dT)(mU)#(mC)#(mA)(fG)(mC)(fA)(mG)(fC)(mA)(fA)(mA) |
| | (mG)(mU)(fC)#(mA)#(mA) |
| MOE357a | (eG)#(eG)#(eC)#(eG)#(eU)#(dG)#(d5C)#(dT)#(dT)#(dG)#(dG)#(d5C)#(d5C) |
| | #(dG)#(dG)#(eG)#(eU)#(eU)#(eG)#(eU) |
| si244_s | (mA)#(mA)#(mU)(fG)(mA)(fU)(mU)(fU)(mC)(fG)(mC)(mC)(mU)(fU)#(mC)#(mA) |
| si244_as | V(mU)#(fG)#(mA)(fA)(fG)(fG)(mC)(fG)(mA)(fA)(mA)(fU)(mC)(fA)#(mU) |
| | #(fU)#(mG)#(mC)#(mC)#(fC)#(mU) |
| si244-MOE357a_s | (mA)#(mA)#(mU)(fG)(mA)(fU)(mU)(fU)(mC)(fG)(mC)(mC)(mU)(fU)#(mC) |
| | #(mA)(PEG4)(dT)(dT)(eG)#(eG)#(eC)#(eG)#(eU)#(dG)#(d5C)#(dT)#(dT) |
| | #(dG)#(dG)#(d5C)#(d5C)#(dG)#(dG)#(eG)#(eU)#(eU)#(eG)#(eU) |
| MOE357a-si244_s | (eG)#(eG)#(eC)#(eG)#(eU)#(dG)#(d5C)#(dT)#(dT)#(dG)#(dG)#(d5C)#(d5C) |
| | #(dG)#(dG)#(eG)#(eU)#(eU)#(eG)#(eU)(dT)(dT)(PEG4)(mA)#(mA)#(mU)(fG) |
| | (mA)(fU)(mU)(fU)(mC)(fG)(mC)(mC)(mU)(fU)#(mC)#(mA) |

### Cd47 and Cd98 Gene Targets

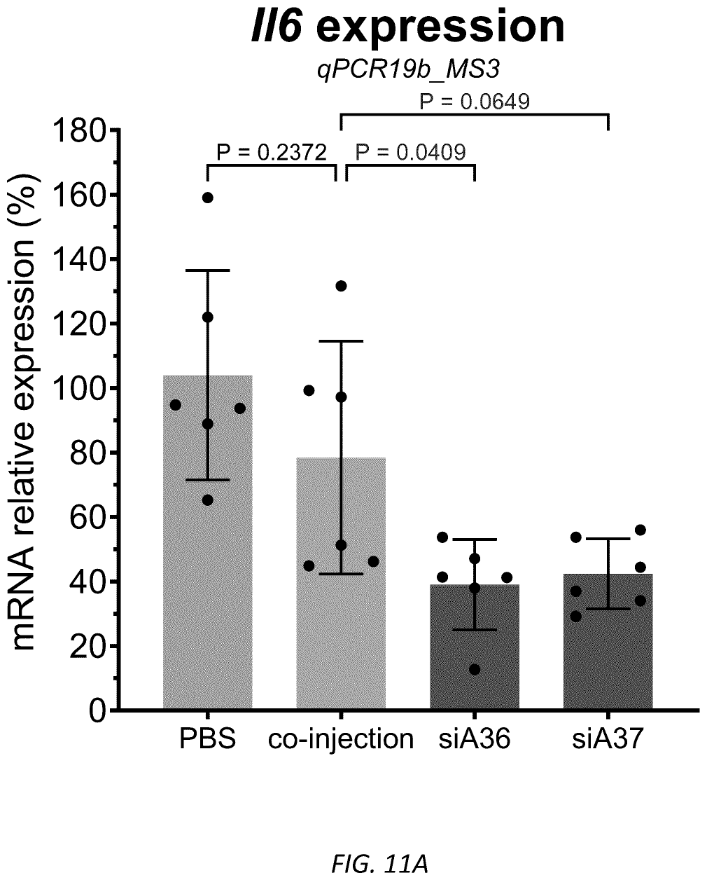

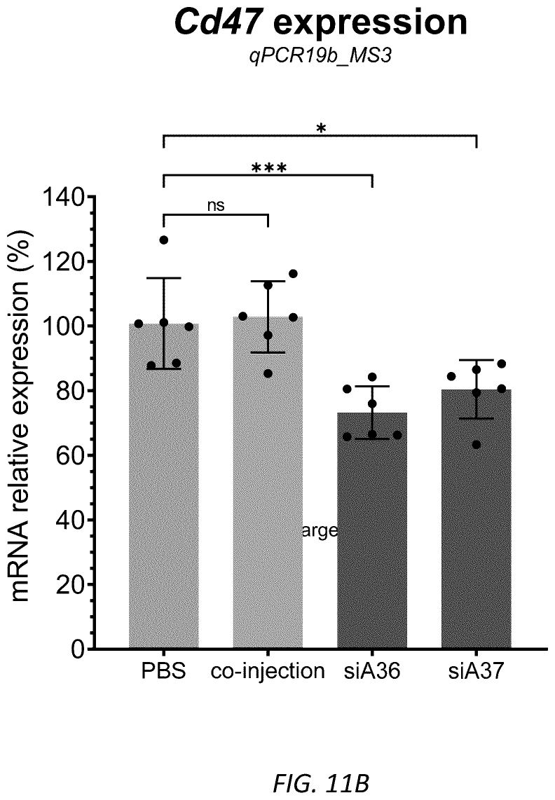

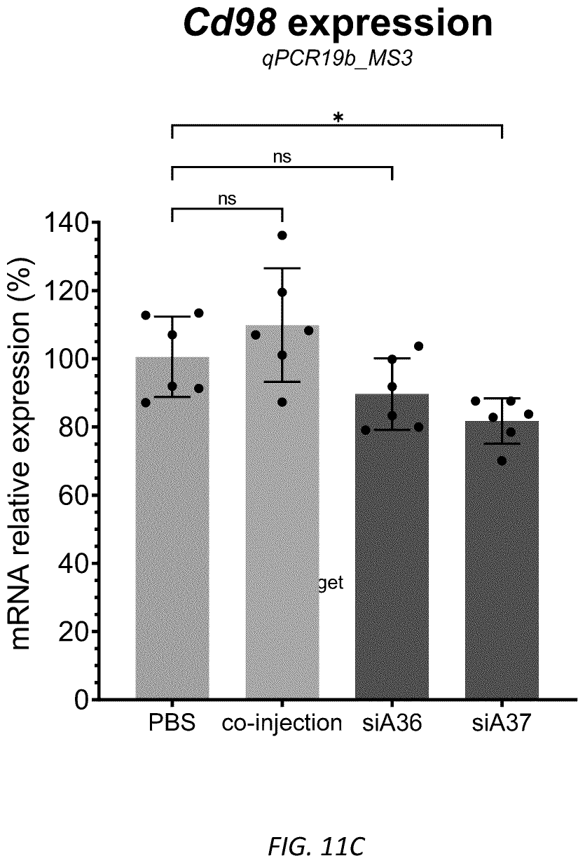

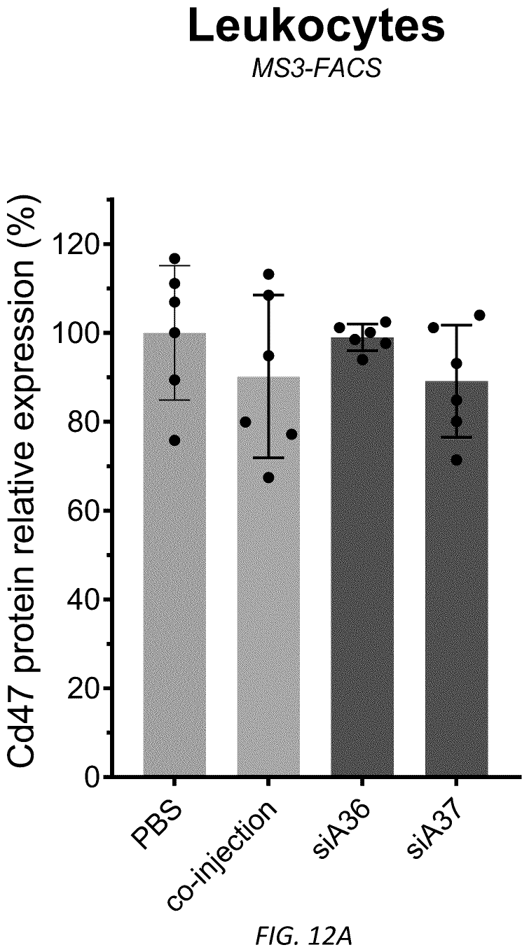

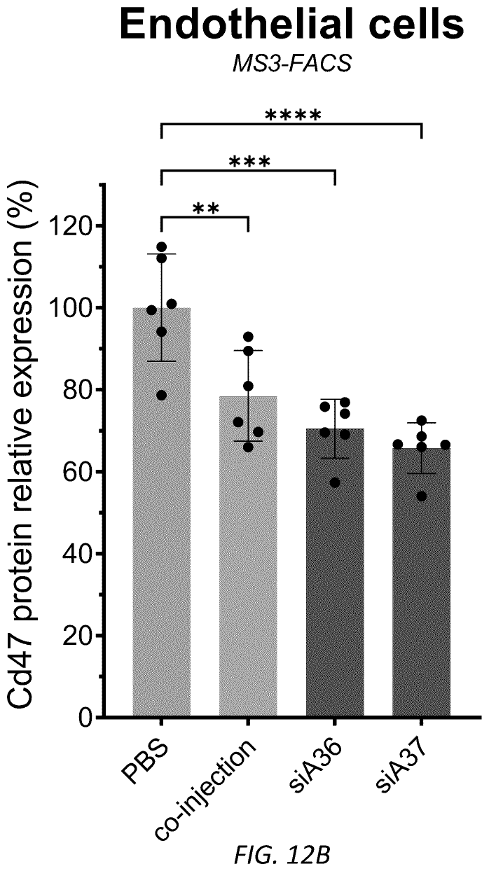

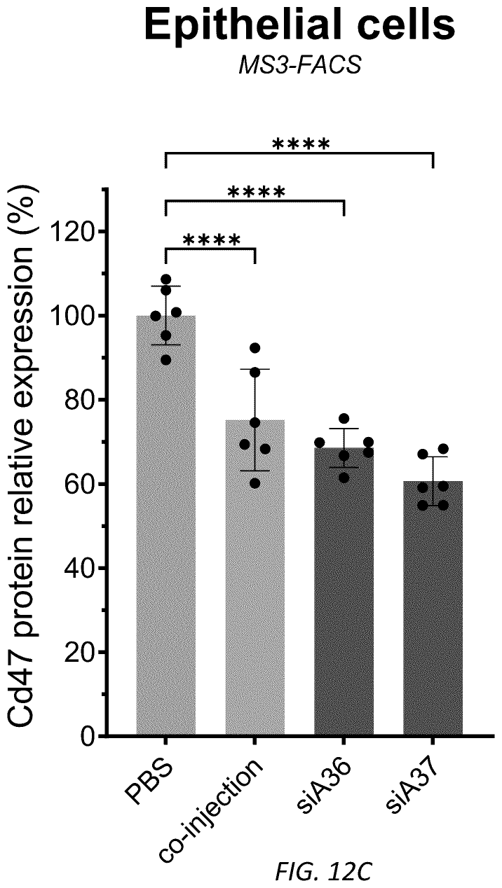

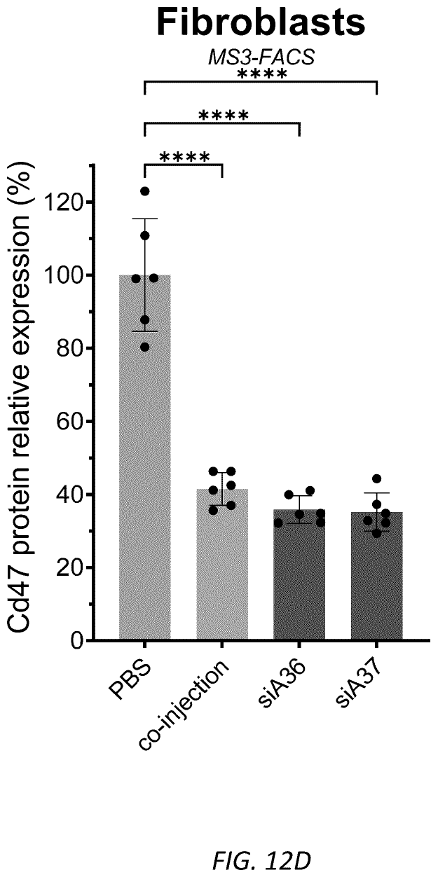

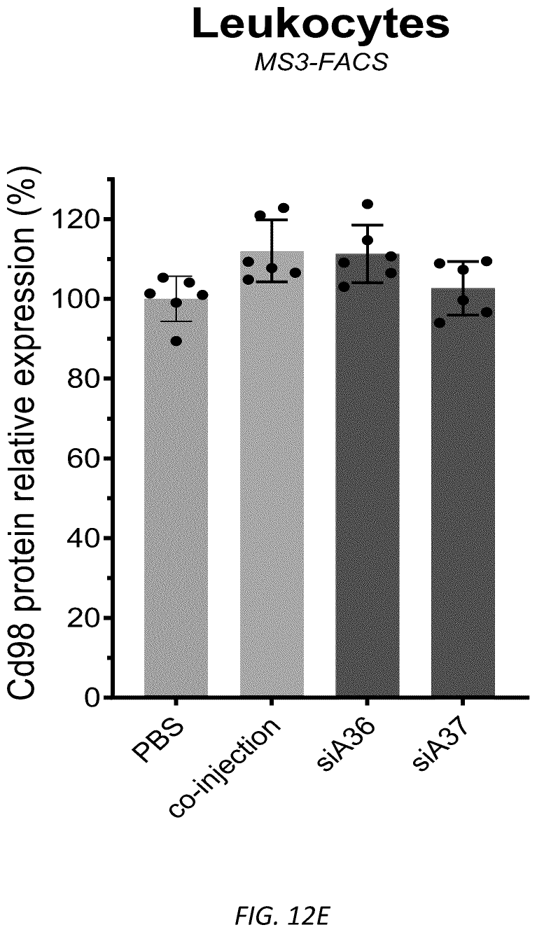

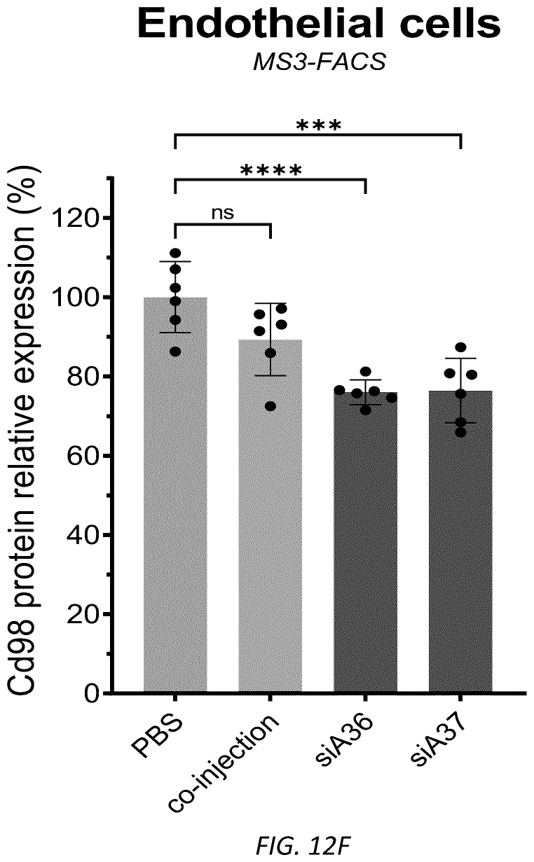

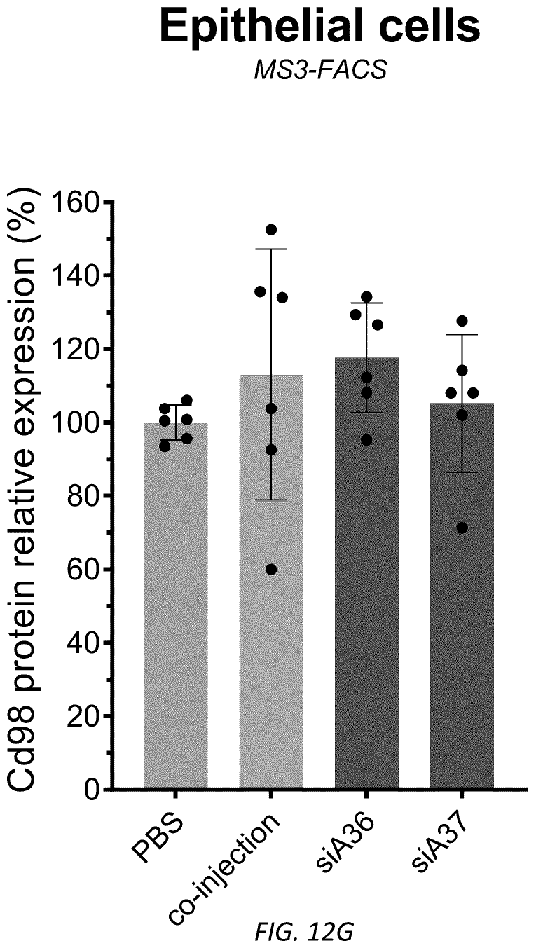

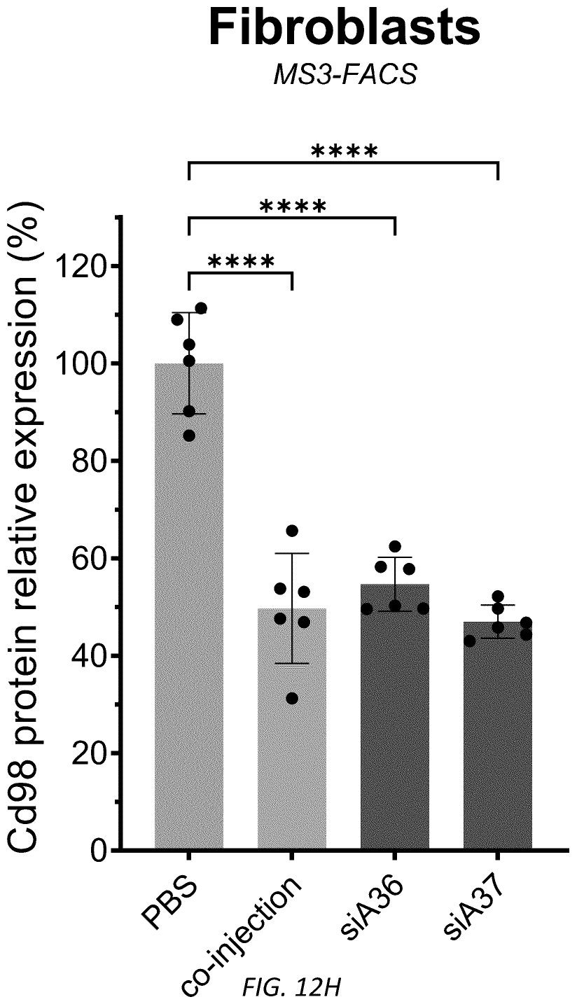

One of several siRNASO formats were administered intratracheally to mice, where the siRNASO targets the silencing of both Cd47 and Cd98 genes (FIGS. 11A-C and FIGS. 12A-H). Intratracheal injection of oligonucleotides was performed on day 1: co-injection (10 nmol siRNA Cd47, 10 nmol ASO Cd98), 10 nmol siA36, and 10 nmol siA37. Mice were sacrificed on day 10 for analysis. Table 7 below describes the various siRNAs and ASOs employed.

TABLE 7
siRNAs and ASO

| Oligo ID | Sequence |
|---|---|
| siA36_s | (mU)#(mC)#(mA)(mC)(mA)(fU)(fA)(fA)(mA)(fU)(mG)(mA)(mU)(mU)#(mA)#(mA |
| | (PEG4)(dT)(dT)(1A)#(1C)#(1C)#(dG)#(dG)#(d5C)#(d5C)#(d5C)#(dG)#(dA)#(dA) |
| | #(dT)#(d5C)#(1T)#(1C)#(1G) |
| siA37_s | (1A)#(1C)#(1C)#(dG)#(dG)#(d5C)#(d5C)#(d5C)#(dG)#(dA)#(dA)#(dT)#(d5C)#(1T) |
| | #(1C)#(1G)(dT)(dT)(PEG4)(mU)#(mC)#(mA)(mC)(mA)(fU)(fA)(fA)(mA)(fU)(mG) |
| | (mA)(mU)(mU)#(mA)#(mA) |
| CD98_6 | (1A)#(1C)#(1C)#(dG)#(dG)#(d5C)#(d5C)#(d5C)#(dG)#(dA)#(dA)#(dT)#(d5C)#(IT) |
| (Slc3a2_1537)_ASO | #(1C)#(1G) |
| Cd47_siRNA_1232_as | (mU)#(fU)#(mA)(mA)(mU)(fC)(mA)(mU)(mU)(mU)(mA)(mU)(mG)(fU)#(mG)#(fA)#(mC) |
| | #(mU)#(mU)#(mU)#(mU) |
| Cd47_siRNA_1232_s | (mU)#(mC)#(mA)(mC)(mA)(fU)(fA)(fA)(mA)(fU)(mG)(mA)(mU)(mU)#(mA)#(mA) |

It was found that siRNASO shows more robust silencing in both targets (Cd47 and Cd98) than the co-injection group in both bulk analysis (FIGS. 11A-C) and specific cell population (Cd47 in Epithelial cells and C₉₈ in endothelial cells). FIG. 11A shows that siA36 and siA37 induced a lower IL6 expression level compared with co-injection. FIG. 11B-C show that siA36 and siA37 induced silencing of both Cd47 (B) and Cd98 (C), while the co-injection group did not. Cd47 silencing analysis in leukocytes (FIG. 12A), endothelial cells (FIG. 12B), epithelial cells (FIG. 12C), and fibroblasts (FIG. 12D), and Cd98 silencing analysis in leukocytes (FIG. 12E), endothelial cells (FIG. 12F), epithelial cells (FIG. 12G), and fibroblasts (FIG. 12H) were performed.

### Lung Tissue

The siRNASO format was tested in the lungs of mice to determine gene silencing efficacy.

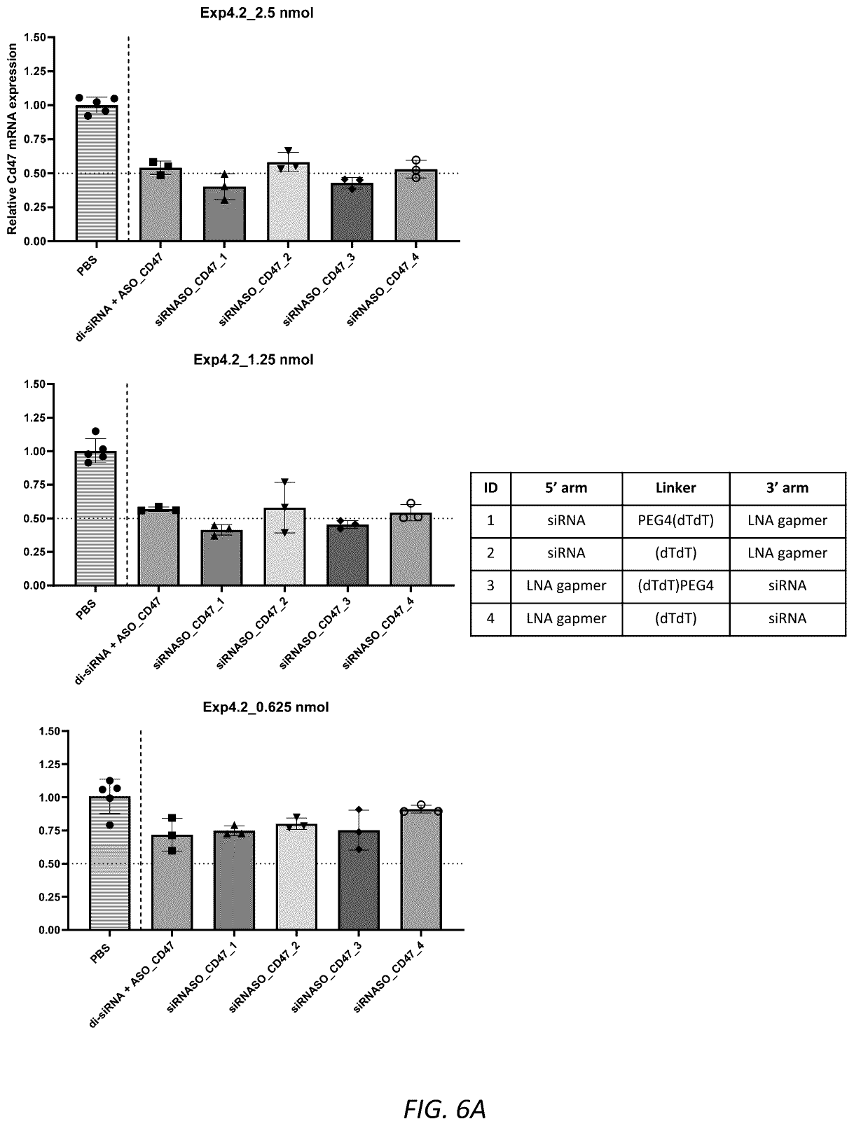

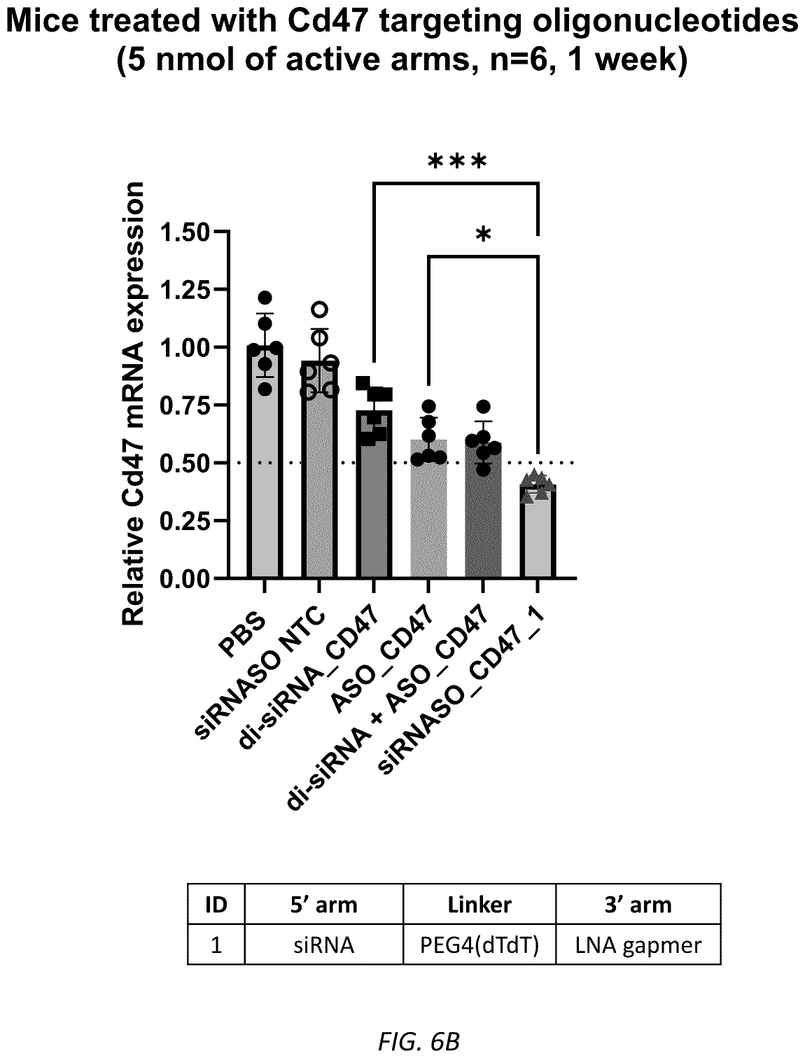

FIG. 6A-B depicts relative RNA expression of Cd47 in mouse lung tissue after intratracheal administration of Cd47-targeting siRNASOs with four different structures at three doses (FIG. 6A, Cd47 expression analyzed at 7 days post administration (n=3); FIG. 6B depicts relative RNA expression of Cd47 in mouse lung tissue after intratracheal administration of Cd47-targeting siRNASO at 5 nmol of active arms (FIG. 6B, Cd47 expression analyzed at 7 days post administration (n=6)). Robust decreases in Cd47 expressions were observed at multiple dose levels with greater silencing achieved by the siRNASO format.

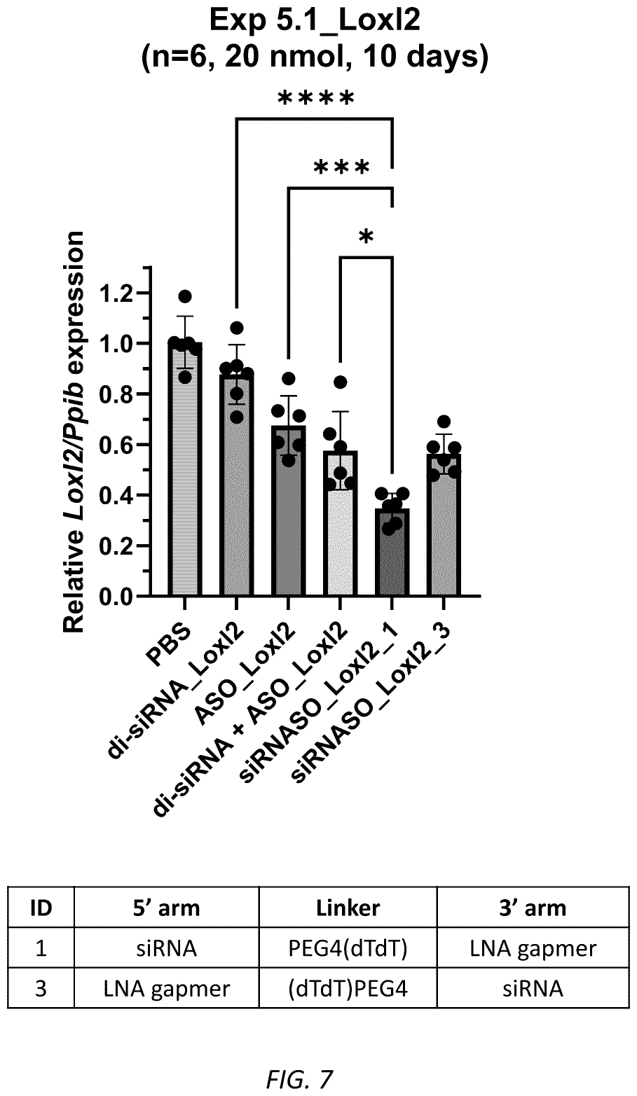

FIG. 7 depicts relative RNA expression of Loxl2 in mouse lung tissue after intratracheal administration of Loxl2-targeting siRNASO at 20 nmol of active arms (FIG. 7, Loxl2 expression analyzed at 10 days post administration (n=6)). As with Cd47 above, Loxl2 expression was reduced more effectively in the siRNASO format.

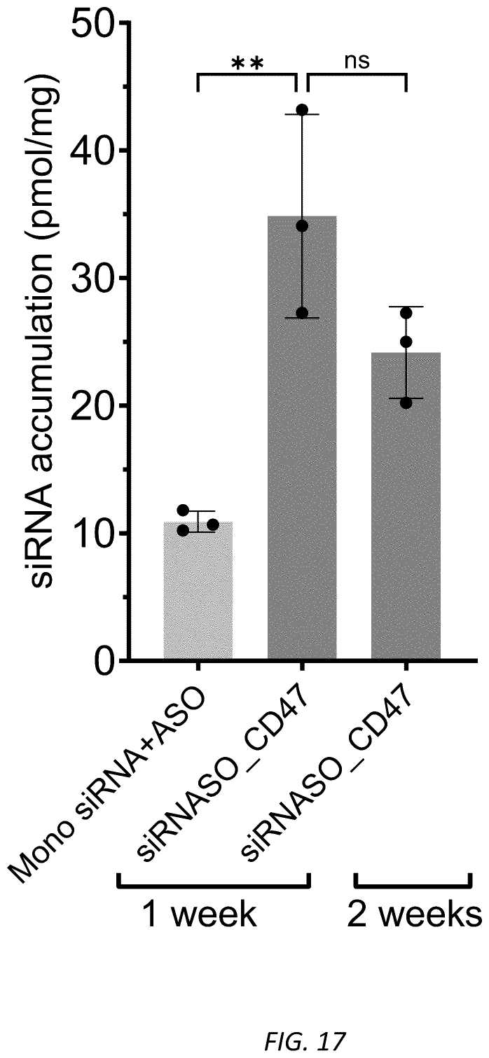

FIG. 17 depicts siRNA accumulation from the siRNASO in the mouse lung after 1 and 2 weeks. Compared against mixed but non-conjugated siRNA comparator group (mono asymmetric siRNA). Administered intratracheal, 15 or 7.5 nmol, n=3, 1 week, PNA hybridization assay, 21/16 antisense strand/sense strand length with cleavable linker. Intratracheal injection of siRNASO significantly improves accumulation in lung relative to a mixed but non-conjugated siRNA comparator group (mono asymmetric siRNA).

### Central Nervous System

The siRNASO format was tested in the CNS of mice to determine gene silencing efficacy.

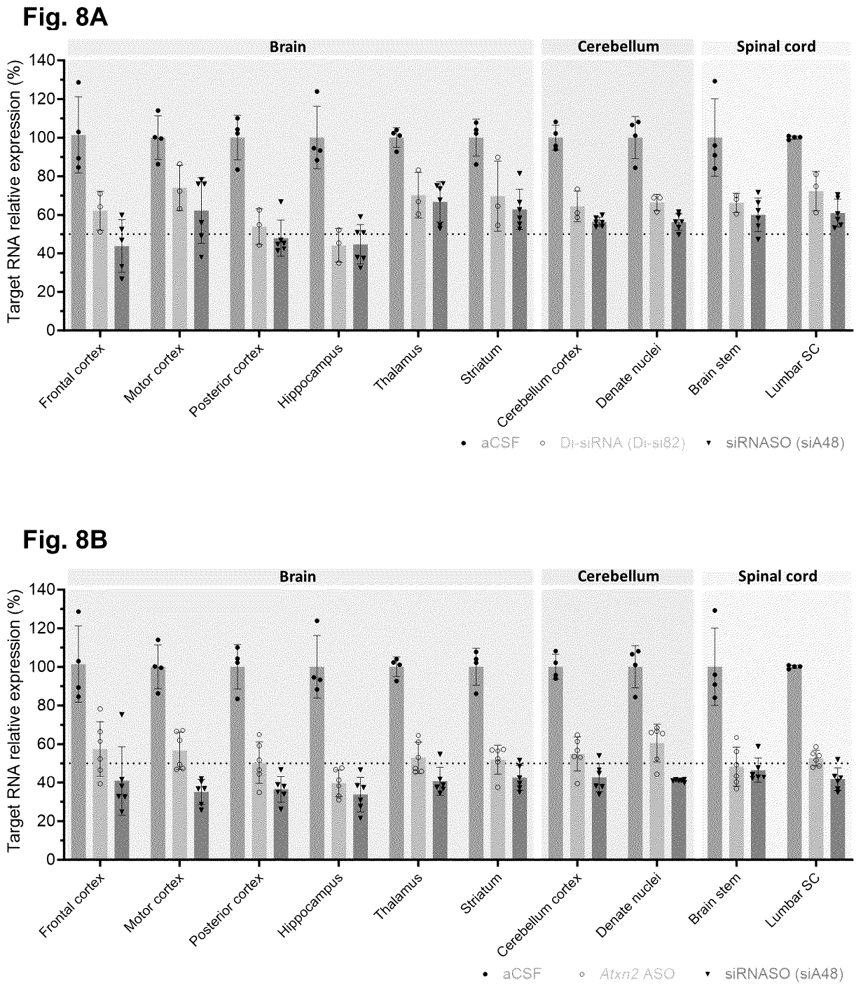

FIG. 8A-B depict relative RNA expression of Atxn2 in the mouse brain following Bi-ICV injection followed by two-week incubation. FIG. 8A compares di-siRNA targeting Atxn2 to an siRNASO with the same siRNA targeting Atxn2. At 200 μg, siRNASO shows either similar or more robust Atxn2 silencing compared with Di-siRNA. FIG. 8B compares an ASO targeting Atxn2 to an siRNASO with an siRNA targeting Atxn2. At 28 nmols of intact molecules, siRNASO shows more robust Atxn2 silencing compared with ASO.

FIG. 10A-B depict relative RNA expression of Atxn2 RNA in the mouse brain following Bi-ICV injection followed by two-week incubation. FIG. 10A depicts the structures of siRNASO tested. FIG. 10B depicts silencing of the Atxn2 RNA in various structures of the mouse brain. The results show that multiple versions of the siRNASO effectively silencing a target gene in the CNS.

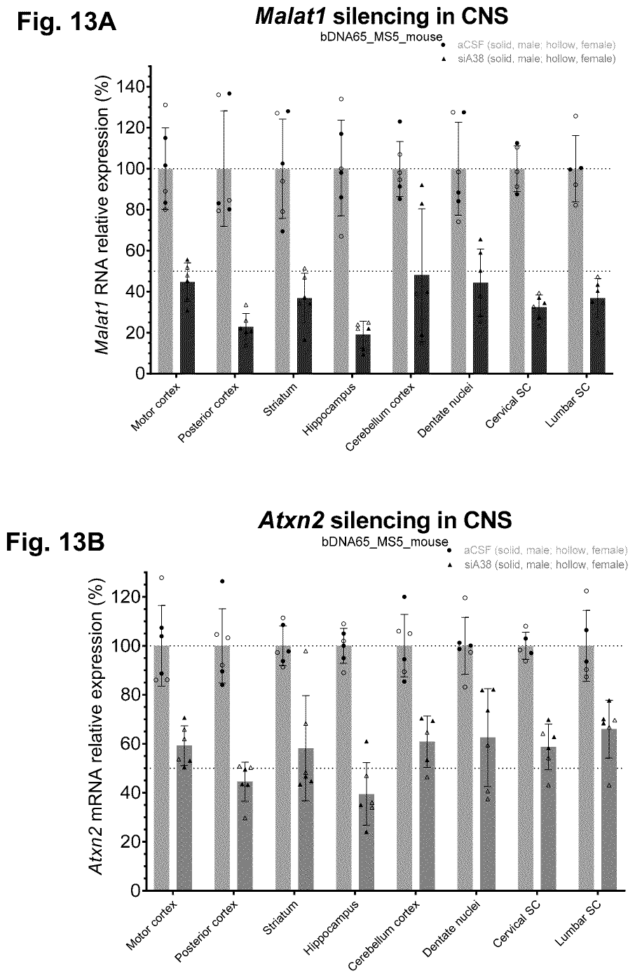

FIG. 13A-B depict relative RNA expression of Malat1 (FIG. 13A) and Atxn2 (FIG. 13B) RNA in the mouse brain following Bi-ICV injection at 20 nmol followed by two-week incubation. Incubation was with an siRNASO with an siRNA targeting Atxn2 and an ASO targeting Malat1. The results show that the siRNASO format potently silences targets through both the ASO arm and the siRNA arm.

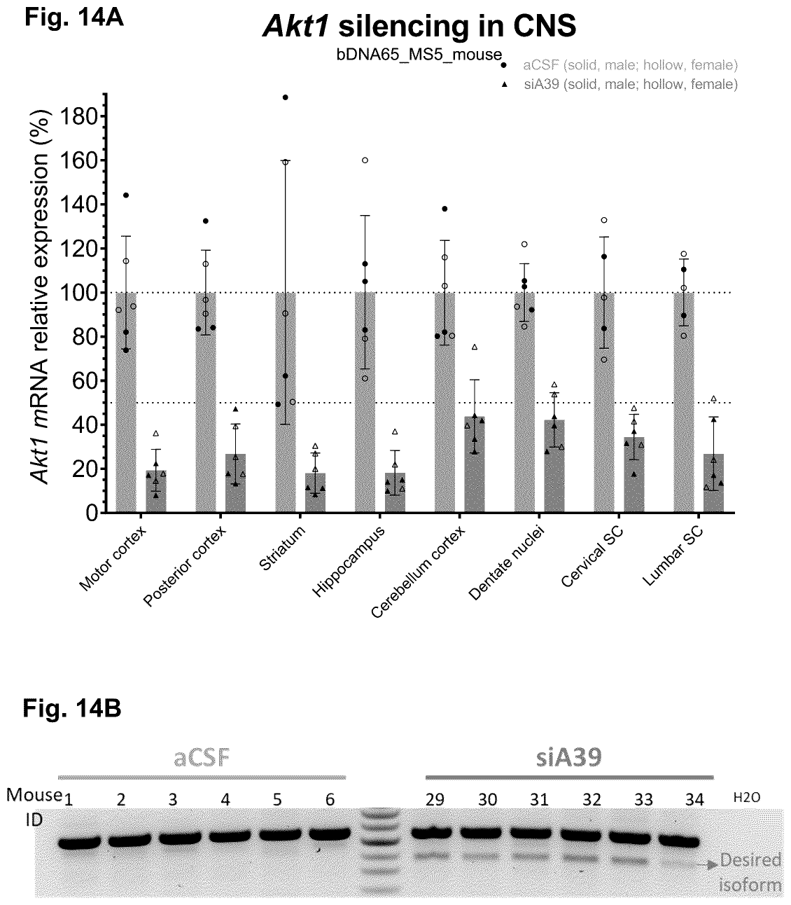

FIG. 14A-B depict silencing & exon skipping by siRNASO (siA39, 33 nmol of intact molecule) in mouse brain. SiRNA arm targeted Akt1 and ASO arm induced exon skipping of Cln3. FIG. 14A depicts the RNA expression of Akt1 that is targeted by the siRNA arm. FIG. 14B depicts PCR result of Cln3 that is targeted by the ASO arm from cDNA samples from the motor cortex of indicated mice. Data was measured in the mouse brain following Bi-ICV injection at 33 nmol followed by two-week incubation. The results show that the siRNASO format can effectively deliver a functional exon-skipping ASO, and not merely a gene silencing ASO with RNAse H activity.

### Safety Profile

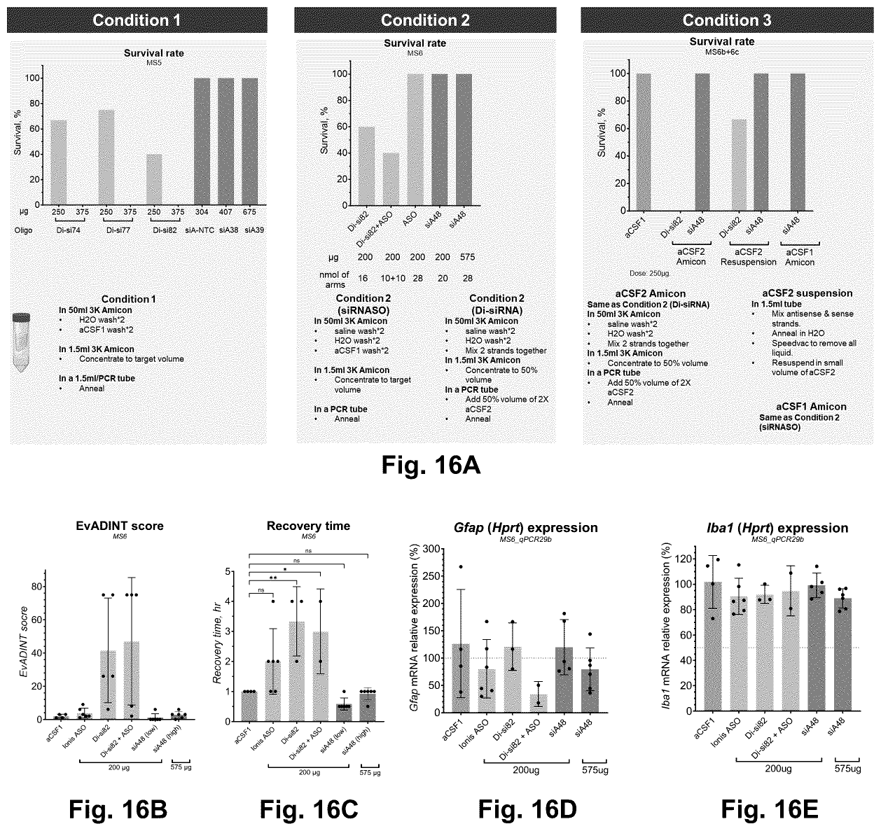

FIG. 16 depicts the short- and long-term safety profile of siRNASO in mouse CNS. FIG. 16A, with doses ranging from 200-675 μg and various preparation methods, siRNASO shows 100% survival in all conditions. FIG. 16B-C, siRNASO shows safe recovery similar to the aCSF1 group. FIG. 16D-E, siRNASO shows no elevated neuroinflammation makers (Gfap, Iba1) at 2 weeks post ICV injection.

### Example 3. SiRNASO Linker Optimization

Most studies described above utilized the PEG4-dTdT linker to attach the siRNA to the ASO. Alternative linker designs were tested for their impact on target gene silencing.

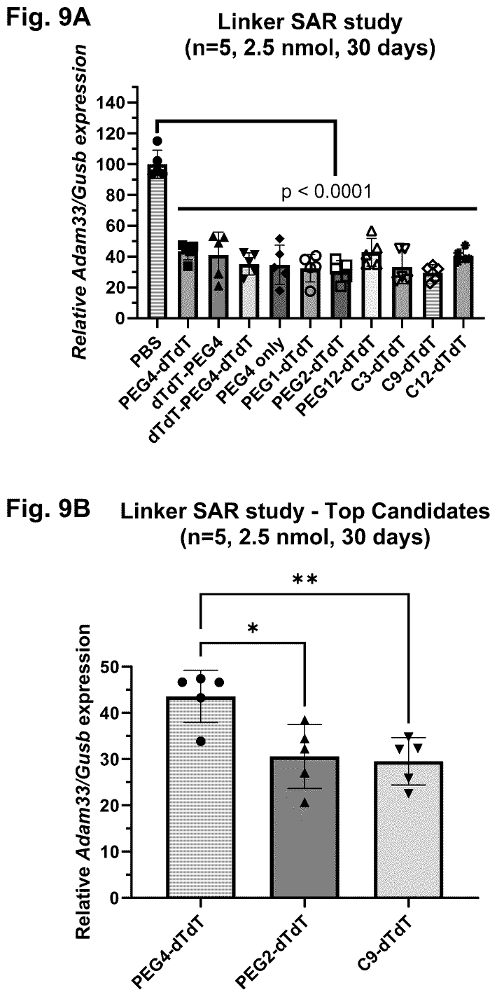

FIG. 9 depicts extensive analysis of linker structures and top candidates that perform significantly better than the parent structure PEG4-dTdT. FIG. 9A depicts relative RNA expression of Adam33 in mouse lung tissue after intratracheal administration of Adam33-targeting siRNASO at 2.5 nmol (Adam33 expression analyzed at 30 days post administration (n=5)). All groups showed significant silencing compared to PBS control group (p<0.0001). Linker structures tested contained various lengths of polyethylene glycol (PEG) and alkyl (C₃=propyl, C₉=nonyl, C₁₂=dodecyl). All linkers contained dTdT di-nucleotide except for PEG4 only. FIG. 9B depicts post hoc analysis of the top two linkers relative to first generation linker PEG4-dTdT.

### Example 4. SiRNASO Gene Silencing and RNA Editing

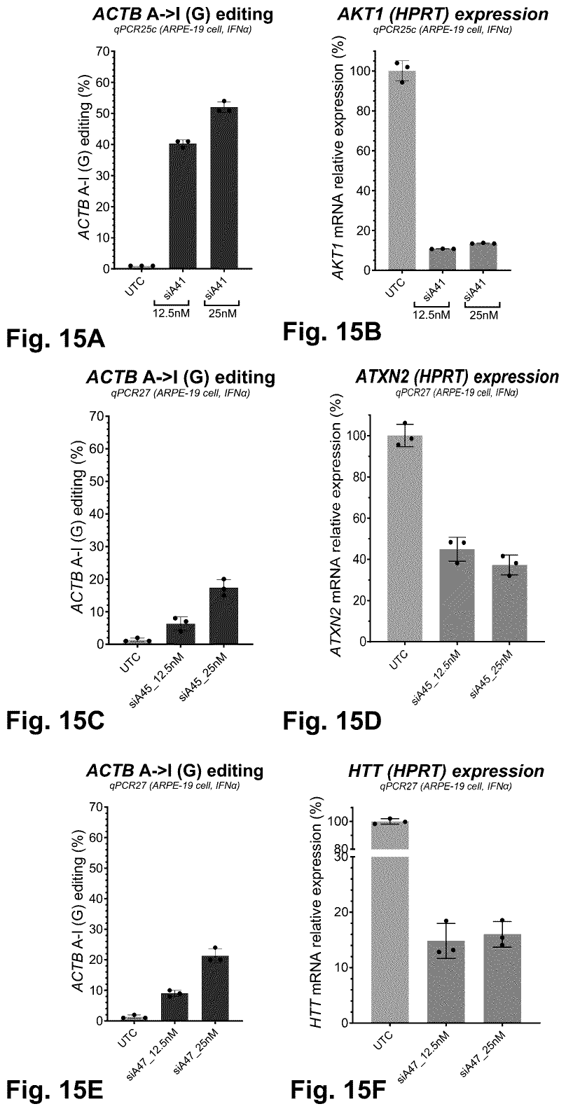

FIG. 15 A-F depicts the silencing & RNA editing by siRNASO transfected in ARPE-19 cells. FIG. 15 A-B, silencing and RNA editing by siRNASO (siA41). ASO arm targets ACTB and siRNA arm targets AKT1. FIG. 15 C-D, silencing and RNA editing by siRNASO (siA45). ASO arm targets ACTB and siRNA arm targets ATXN2. FIG. 15 E-F, silencing and RNA editing by siRNASO (siA47). ASO arm targets ACTB and siRNA arm targets HTT.

TABLE 8
Sequences

| Oligo ID | sequence |
|---|---|
| siA-NTC_AS | V(mU)#(fA)#(mA)(fU)(fC)(fG)(mU)(fA)(mU)(fU)(mU)(fG)(mU)(fC)#(mA) |
| | #(fA)#(mU)#(mC)#(mA)#(mU)#(fU) |
| siA-NTC_S | (mU)#(mU)#(mG)(fA)(mC)(fA)(mA)(fA)(mU)(fA)(mC)(mG)(mA)(fU)#(mU) |
| | #(mA)(Tetra-EG)(dT)(dT)(eC)#(eC)(eU)(eU)(eC)(d5C)#(d5C)#(dT) |
| | #(dG)#(dA)#(dA)#(dG)#(dG)#(dT)#(dT)#(eC)(eC)(EU)#(eC)#(eC) |
| siA38_AS | V(mU)#(fA)#(mU)(fA)(fC)(fA)(mA)(fA)(mU)(fU)(mC)(fU)(mA)(fG)#(mG) |
| | #(fC)#(mC)#(mA)#(mC)#(fU)#(mU) |
| siA38_S | (mG)#(mC)#(mC)(fU)(mA)(fG)(mA)(fA)(mU)(fU)(mU)(mG)(mU)(fA)#(mU) |
| | #(mA)(Tetra-EG)(dT)(dT)(eG)#(eC)(eC)(eA)(eG)(dG)#(d5C)#(dT)#(dG) |
| | #(dG)#(dT)#(dT)#(dA)#(dT)#(dG)#(eA)(eC)(eU)#(eC)#(eA) |
| siA39_AS | V(mU)#(fC)#(mA)(fA)(fA)(fC)(mU)(fC)(mG)(fU)(mU)(fC)(mA)(fU)#(mG) |
| | #(fG)#(mU)#(mC)#(mA)#(mC)#(fU) |
| siA39_S | (mC)#(mC)#(mA)(fU)(mG)(fA)(mA)(fC)(mG)(fA)(mG)(mU)(mU)(fU)#(mG) |
| | #(mA)(Tetra-EG)(dT)(dT)(eG)#(eA)#(eC)#(eC)#(eU)#(eG)#(eU)#(eA) |
| | #(eA)#(eG)#(eG)#(eC)#(eA)#(eG)#(eC)#(eA)#(eA)#(eG) |
| siA41_AS | (5′P)(mU)#(fC)#(mA)(fA)(fA)(fC)(mU)(fC)(mG)(fU)(mU)(fC)(mA)(fU) |
| | #(mG)#(fG)#(mU)#(mC)#(mA)#(mC)#(fU) |
| siA41_S | (mC)#(mC)#(mA)(fU)(mG)(fA)(mA)(fC)(mG)(fA)(mG)(mU)(mU)(fU)#(mG) |
| | #(mA)(Tetra-EG)(dT)(dT)(fA)#(fC)#(fA)#(fU)#(fA)#(fA)#(fU)#(fU) |
| | #(fU)#(fA)#(fG)#(fA)#(fC)#(fG)#(fU)#(mA)#(mA)#(mG)#(mC)#(mA) |
| | #(mA)#(mU)#(mG)#(fC)#(d5C)#(dA)#(mU)#(mC)#(mA)#(mC) |
| siA45_AS | (5′P)(mU)#(fA)#(mU)(fA)(fC)(fA)(mA)(fA)(mU)(fU)(mC)(fU)(mA)(fG) |
| | #(mG)#(fC)#(mC)#(mA)#(mC)#(fU)#(mU) |
| siA45_S | (mG)#(mC)#(mC)(fU)(mA)(fG)(mA)(fA)(mU)(fU)(mU)(mG)(mU)(fA)#(mU) |
| | #(mA)(Tetra-EG)(dT)(dT)(fA)#(fC)#(fA)#(fU)#(fA)#(fA)#(fU)#(fU) |
| | #(fU)#(fA)#(fG)#(fA)#(fC)#(fG)#(fU)#(mA)#(mA)#(mG)#(mC)#(mA)#(mA) |
| | #(mU)#(mG)#(fC)#(d5C)#(dA)#(mU)#(mC)#(mA)#(mC) |
| siA47_AS | P(mU)#(fU)#(mA)(fA)(fU)(fC)(mU)(fC)(mU)(fU)(mU)(fA)(mC)(fU)#(mG) |
| | #(fA)#(mU)#(mA)#(mU)#(mA)#(fU) |
| siA47_S | (mU)#(mC)#(mA)(fG)(mU)(fA)(mA)(fA)(mG)(fA)(mG)(mA)(mU)(fU)#(mA) |
| | #(mA)(Tetra-EG)(dT)(dT)(fA)#(fC)#(fA)#(fU)#(fA)#(fA)#(fU)#(fU) |
| | #(fU)#(fA)#(fG)#(fA)#(fC)#(fG)#(fU)#(mA)#(mA)#(mG)#(mC)#(mA) |
| | #(mA)#(mU)#(mG)#(fC)#(d5C)#(dA)#(mU)#(mC)#(mA)#(mC) |
| Di-si74_AS | V(mU)#(fU)#(mG)(fA)(fA)(fA)(mU)(fC)(mU)(fG)(mA)(fA)(mG)(fU)#(mG) |
| | #(fU)#(mG)#(mA)#(mG)#(fA)#(mU) |
| Di-si74_S | (mA)#(mC)#(mA)(fC)(mU)(fU)(mC)(fA)(mG)(fA)(mU)(mU)(mU)(fC)#(mA) |
| | #(mA)-DIO |
| Di-si77_AS | V(mU)#(fU)#(mA)(fA)(fU)(fU)(mU)(fG)(mG)(fG)(mA)(fC)(mA)(fU)#(mG) |
| | #(fC)#(mA)#(mU)#(mA)#(fC)#(mU) |
| Di-si77_S | (mG)#(mC)#(mA)(fU)(mG)(fU)(mC)(fC)(mC)(fA)(mA)(mA)(mU)(fU)#(mA) |
| | #(mA)-DIO |
| Di-si82_AS | V(mU)#(fA)#(mU)(fA)(fC)(fA)(mA)(fA)(mU)(fU)(mC)(fU)(mA)(fG)#(mG) |
| | #(fC)#(mC)#(mA)#(mC)#(fU)#(mU) |
| Di-si82_S | (mG)#(mC)#(mC)(fU)(mA)(fG)(mA)(fA)(mU)(fU)(mU)(mG)(mU)(fA)#(mU) |
| | #(mA)-DIO |
| siA48_AS | V(mU)#(fA)#(mU)(fA)(fC)(fA)(mA)(fA)(mU)(fU)(mC)(fU)(mA)(fG)#(mG) |
| | #(fC)#(mC)#(mA)#(mC)#(fU)#(mU) |
| siA48_S | (mG)#(mC)#(mC)(fU)(mA)(fG)(mA)(fA)(mU)(fU)(mU)(mG)(mU)(fA)#(mU) |
| | #(mA)(Tetra-EG)(dT)(dT)(eC)#(eU)(eU)(eC)(eA)(d5C)#(dA)#(dT)#(dT) |
| | #(dT)#(d5C)#(dG)#(dA)#(dT)#(d5C)#(eC)(eA)(eA)#(eC)#(eA) |
| Atxn2 ASO | (eC)#(eU)(eU)(eC)(eA)(d5C)#(dA)#(dT)#(dT)#(dT)#(d5C)#(dG)#(dA) |
| | #(dT)#(d5C)#(eC)(eA)(eA)#(eC)#(eA) |
| siA50_AS | same as siA48_AS |
| siA50_S | (mG)#(mU)#(mG)(mG)(mC)(mC)(fU)(mA)(fG)(mA)(fA)(mU)(fU)(mU)(mG) |
| | (mU)(fA)#(mU)#(mA)(Tetra-EG)(dT)(dT)(eC)#(eU)(eU)(eC)(eA)(d5C) |
| | #(dA)#(dT)#(dT)#(dT)#(d5C)#(dG)#(dA)#(dT)#(d5C)#(eC)(eA)(eA) |
| | #(eC)#(eA) |
| siA51_AS | same as siA48_AS |
| siA51_S | (mG)#(mC)#(mC)(fU)(mA)(fG)(mA)(fA)(mU)(fU)(mU)(mG)(mU)(fA)#(mU) |
| | #(mA)(Tetra-EG)(dT)(dT)(mU)#(mG)#(mU)(rU)(rG)(rG)(rA)(rU)(rC) |
| | (rG)(rA)(rA)(rA)(rU)(rG)(rU)(rG)(mA)#(mA)#(mG) |
| Di-siA48_AS | same as siA48_AS |
| Di-siA48_S | (mG)#(mC)#(mC)(fU)(mA)(fG)(mA)(fA)(mU)(fU)(mU)(mG)(mU)(fA)#(mU) |
| | #(mA)(Tetra-EG)(dT)(dT)(eC)#(eU)(eU)(eC)(eA)(d5C)#(dA)#(dT) |
| | #(dT)#(dT)#(d5C)#(dG)#(dA)#(dT)#(d5C)#(eC)(eA)(eA)#(eC)#(eA)(dT) |
| | (dT)-DIO |
| di-siRNA_Loxl2_as | V(mU)#(mG)#(fU)(fU)(fA)(mG)(fA)(mG)(fA)(mA)(fA)(mU)(fC)(mU)#(fG) |
| | #(mA)#(mU)#(mU)#(mC)#(mU)#(mU) |
| di-siRNA_Loxl2_s | (mC)#(mA)#(mG)(fA)(mU)(fU)(mU)(fC)(mU)(fC)(mU)(mA)(mA)(fC)#(mA) |
| | #(mA)-DIO |
| ASO_Loxl2 | (1T)#(1C)#(1A)#(d5C)#(dA)#(dG)#(dG)#(dG)#(dT)#(d5C)#(d5C)#(d5C) |
| | #(dG)#(1G)#(1G)#(IT) |
| siRNASO_Loxl2_1_s | (mC)#(mA)#(mG)(fA)(mU)(fU)(mU)(fC)(mU)(fC)(mU)(mA)(mA)(fC)#(mA) |
| | #(mA)(Tetra-EG)(dT)(dT)(IT)#(IC)#(IA)#(d5C)#(dA)#(dG)#(dG)#(dG) |
| | #(dT)#(d5C)#(d5C)#(d5C)#(dG)#(1G)#(1G)#(1T) |
| siRNASO_Loxl2_3_s | (1T)#(1C)#(1A)#(d5C)#(dA)#(dG)#(dG)#(dG)#(dT)#(d5C)#(d5C)#(d5C) |
| | #(dG)#(1G)#(1G)#(1T)(dT)(dT)(Tetra-EG)(mC)#(mA)#(mG)(fA)(mU)(fU) |
| | (mU)(fC)(mU)(fC)(mU)(mA)(mA)(fC)#(mA)#(mA) |
| siRNASO NTC_as | V(mU)#(fA)#(mA)(fU)(fC)(fG)(mU)(fA)(mU)(fU)(mU)(fG)(mU)(fC)#(mA) |
| | #(fA)#(mU)#(mC)#(mA)#(fU)#(mU) |
| siRNASO NTC_s | (mU)#(mU)#(mG)(fA)(mC)(fA)(mA)(fA)(mU)(fA)(mC)(mG)(mA)(fU)#(mU) |
| | #(mA)(Tetra-EG)(dT)(dT)(1T)#(1C)#(1G)#(dT)#(dA)#(dT)#(dT)#(dT) |
| | #(dG)#(dT)#(d5C)#(dA)#(1A)#(1T)#(1C) |
| di-siRNA_CD47_as | V(mU)#(fU)#(mA)(mA)(mU)(fC)(mA)(mU)(mU)(mU)(mA)(mU)(mG)(fU)#(mG) |
| | #(fA)#(mC)#(mU)#(mU)#(mU)#(mU) |
| di-siRNA_CD47_s | (mU)#(mC)#(mA)(mC)(mA)(fU)(fA)(fA)(mA)(fU)(mG)(mA)(mU)(mU)#(mA) |
| | #(mA)-DIO |
| ASO_CD47 | (1G)#(1T)#(1G)#(d5C)#(dT)#(dT)#(dG)#(dG)#(d5C)#(dG)#(dA)#(dG) |
| | #(dT)#(1C)#(1T)#(1C) |
| siRNASO_CD47_1_s | (mU)#(mC)#(mA)(mC)(mA)(fU)(fA)(fA)(mA)(fU)(mG)(mA)(mU)(mU)#(mA) |
| | #(mA)(Tetra-EG)(dT)(dT)(1G)#(1T)#(1G)#(d5C)#(dT)#(dT)#(dG)#(dG) |
| | #(d5C)#(dG)#(dA)#(dG)#(dT)#(1C)#(1T)#(1C) |
| siRNASO_CD47_2_s | (mU)#(mC)#(mA)(mC)(mA)(fU)(fA)(fA)(mA)(fU)(mG)(mA)(mU)(mU)#(mA) |
| | #(mA)(dT)(dT)(1G)#(1T)#(1G)#(d5C)#(dT)#(dT)#(dG)#(dG)#(d5C)#(dG) |
| | #(dA)#(dG)#(dT)#(1C)#(1T)#(1C) |
| siRNASO_CD47_3_s | (IG)#(IT)#(1G)#(d5C)#(dT)#(dT)#(dG)#(dG)#(d5C)#(dG)#(dA)#(dG) |
| | #(dT)#(IC)#(IT)#(IC)(dT)(dT)(Tetra-EG)(mU)#(mC)#(mA)(mC)(mA)(fU) |
| | (fA)(fA)(mA)(fU)(mG)(mA)(mU)(mU)#(mA)#(mA) |
| siRNASO_CD47_4_s | (1G)#(1T)#(1G)#(d5C)#(dT)#(dT)#(dG)#(dG)#(d5C)#(dG)#(dA)#(dG) |
| | #(dT)#(1C)#(1T)#(1C)(dT)(dT)(mU)#(mC)#(mA)(mC)(mA)(fU)(fA)(fA) |
| | (mA)(fU)(mG)(mA)(mU)(mU)#(mA)#(mA) |
| PEG4-dTdT_as | V(mU)#(fU)#(mG)(fA)(fC)(fU)(mU)(fU)(mG)(fC)(mU)(fG)(mC)(fU) |
| | #(mG)#(fA)#(mG)#(mC)#(mA)#(fC)#(mU) |
| PEG4-dTdT_s | (mU)#(mC)#(mA)(fG)(mC)(fA)(mG)(fC)(mA)(fA)(mA)(mG)(mU)(fC)#(mA) |
| | #(mA)(PEG4)(dT)(dT)(IT)#(1A)#(1A)#(dG)#(d5C)#(dT)#(d5C)#(dA)#(dG) |
| | #(dA)#(dG)#(dT)#(IT)#(IC)#(1G) |
| dTdT-PEG4_s | (mU)#(mC)#(mA)(fG)(mC)(fA)(mG)(fC)(mA)(fA)(mA)(mG)(mU)(fC)#(mA) |
| | #(mA)(dT)(dT)(Tetra-EG)(1T)#(1A)#(1A)#(dG)#(d5C)#(dT)#(d5C)#(dA) |
| | #(dG)#(dA)#(dG)#(dT)#(1T)#(1C)#(1G) |
| dTdT-PEG4-dTdT_s | (mU)#(mC)#(mA)(fG)(mC)(fA)(mG)(fC)(mA)(fA)(mA)(mG)(mU)(fC)#(mA) |
| | #(mA)(dT)(dT)(Tetra-EG)(dT)(dT)(1T)#(1A)#(1A)#(dG)#(d5C)#(dT) |
| | #(d5C)#(dA)#(dG)#(dA)#(dG)#(dT)#(1T)#(1C)#(1G) |
| PEG4 only_s | (mU)#(mC)#(mA)(fG)(mC)(fA)(mG)(fC)(mA)(fA)(mA)(mG)(mU)(fC)#(mA) |
| | #(mA)(Tetra-EG)(IT)#(1A)#(1A)#(dG)#(d5C)#(dT)#(d5C)#(dA)#(dG) |
| | #(dA)#(dG)#(dT)#(1T)#(1C)#(1G) |
| PEG1-dTdT_s | (mU)#(mC)#(mA)(fG)(mC)(fA)(mG)(fC)(mA)(fA)(mA)(mG)(mU)(fC) |
| | #(mA)#(mA)(PEG1)(dT)(dT)(1T)#(1A)#(1A)#(dG)#(d5C)#(dT)#(d5C) |
| | #(dA)#(dG)#(dA)#(dG)#(dT)#(1T)#(1C)#(1G) |
| PEG2-dTdT_s | (mU)#(mC)#(mA)(fG)(mC)(fA)(mG)(fC)(mA)(fA)(mA)(mG)(mU)(fC)#(mA) |
| | #(mA)(PEG2)(dT)(dT)(IT)#(1A)#(1A)#(dG)#(d5C)#(dT)#(d5C)#(dA)#(dG) |
| | #(dA)#(dG)#(dT)#(1T)#(1C)#(1G) |
| PEG12-dTdT_s | (mU)#(mC)#(mA)(fG)(mC)(fA)(mG)(fC)(mA)(fA)(mA)(mG)(mU)(fC)#(mA) |
| | #(mA)(HEG)(dT)(dT)(1T)#(1A)#(1A)#(dG)#(d5C)#(dT)#(d5C)#(dA)#(dG) |
| | #(dA)#(dG)#(dT)#(1T)#(1C)#(1G) |
| C3-dTdT_s | (mU)#(mC)#(mA)(fG)(mC)(fA)(mG)(fC)(mA)(fA)(mA)(mG)(mU)(fC)#(mA) |
| | #(mA)(C3)(dT)(dT)(1T)#(1A)#(1A)#(dG)#(d5C)#(dT)#(d5C)#(dA)#(dG) |
| | #(dA)#(dG)#(dT)#(1T)#(1C)#(1G) |
| C9-dTdT_s | (mU)#(mC)#(mA)(fG)(mC)(fA)(mG)(fC)(mA)(fA)(mA)(mG)(mU)(fC)#(mA) |
| | #(mA)(C9)(dT)(dT)(1T)#(1A)#(1A)#(dG)#(d5C)#(dT)#(d5C)#(dA)#(dG) |
| | #(dA)#(dG)#(dT)#(1T)#(1C)#(1G) |
| C12-dTdT_s | (mU)#(mC)#(mA)(fG)(mC)(fA)(mG)(fC)(mA)(fA)(mA)(mG)(mU)(fC)#(mA) |
| | #(mA)(C12)(dT)(dT)(1T)#(1A)#(1A)#(dG)#(d5C)#(dT)#(d5C)#(dA)#(dG) |
| | #(dA)#(dG)#(dT)#(1T)#(1C)#(1G) |

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Atwell et al. J. Mol. Biol. 1997, 270: 26-35;
Ausubel et al. (eds.), CURRENT PROTOCOLS IN MOLECULAR BIOLOGY, John Wiley &Sons, N Y (1993);
Ausubel, F. M. et al. eds., SHORT PROTOCOLS IN MOLECULAR BIOLOGY (4th Ed. 1999) John Wiley & Sons, NY. (ISBN 0-471-32938-X);
CONTROLLED DRUG BIOAVAILABILITY, DRUG PRODUCT DESIGN AND PERFORMANCE, Smolen and Ball (eds.), Wiley, New York (1984);
Giege, R. and Ducruix, A. Barrett, CRYSTALLIZATION OF NUCLEIC ACIDS AND PROTEINS, a Practical Approach, 2nd ea., pp. 20 1-16, Oxford University Press, New York, New York, (1999);
Goodson, in MEDICAL APPLICATIONS OF CONTROLLED RELEASE, vol. 2, pp. 115-138 (1984);
Hammerling, et al., in: MONOCLONAL ANTIBODIES AND T-CELL HYBRIDOMAS 563-681 (Elsevier, N.Y., 1981;
Harlow et al., ANTIBODIES: A LABORATORY MANUAL, (Cold Spring Harbor Laboratory Press, 2nd ed. 1988);
Kabat et al., SEQUENCES OF PROTEINS OF IMMUNOLOGICAL INTEREST (National Institutes of Health, Bethesda, Md. (1987) and (1991);
Kabat, E. A., et al. (1991) SEQUENCES OF PROTEINS OF IMMUNOLOGICAL INTEREST, Fifth Edition, U.S. Department of Health and Human Services, NIH Publication No. 91-3242;
Kontermann and Dubel eds., ANTIBODY ENGINEERING (2001) Springer-Verlag. New York. 790 pp. (ISBN 3-540-41354-5).
Kriegler, Gene Transfer and Expression, A Laboratory Manual, Stockton Press, N Y (1990);
Lu and Weiner eds., CLONING AND EXPRESSION VECTORS FOR GENE FUNCTION ANALYSIS (2001) BioTechniques Press. Westborough, MA. 298 pp. (ISBN 1-881299-21-X).
MEDICAL APPLICATIONS OF CONTROLLED RELEASE, Langer and Wise (eds.), CRC Pres., Boca Raton, Fla. (1974);
Old, R. W. & S. B. Primrose, PRINCIPLES OF GENE MANIPULATION: AN INTRODUCTION TO GENETIC ENGINEERING (3d Ed. 1985) Blackwell Scientific Publications, Boston. Studies in Microbiology; V.2:409 pp. (ISBN 0-632-01318-4).
Sambrook, J. et al. eds., MOLECULAR CLONING: A LABORATORY MANUAL (2d Ed. 1989) Cold Spring Harbor Laboratory Press, NY. Vols. 1-3. (ISBN 0-87969-309-6).
SUSTAINED AND CONTROLLED RELEASE DRUG DELIVERY SYSTEMS, J. R. Robinson, ed., Marcel Dekker, Inc., New York, 1978
Winnacker, E. L. FROM GENES TO CLONES: INTRODUCTION TO GENE TECHNOLOGY (1987) VCH Publishers, NY (translated by Horst Ibelgaufts). 634 pp. (ISBN 0-89573-614-4).

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A compound comprising:
a) an siRNA comprising a sense strand and an antisense strand, and
b) an antisense oligonucleotide (ASO),
wherein the siRNA is attached to the ASO with a linker.

2. The compound of claim 1, wherein the linker is a non-nucleotide linker.

3. The compound of claim 2, wherein the non-nucleotide linker comprises an ethylene glycol chain, an alkyl chain, a peptide, an amide, a carbamate, or a combination thereof.

4. The compound of claim 3, wherein the non-nucleotide linker comprises the alkyl chain and the alkyl chain is a C2 to C15 alkyl chain or a propyl (C3), nonyl (C9), or dodecyl (C12) chain.

5. (canceled)

6. The compound of claim 3, wherein the non-nucleotide linker comprises the ethylene glycol chain and the ethylene glycol chain comprises 1-15 ethylene glycol units or a 1 (PEG1), 2 (PEG2), 4 (PEG4), or 12 (PEG12) ethylene glycol units.

7-8. (canceled)

9. The compound of claim 1, wherein the linker comprises a nucleotide linker.

10-11. (canceled)

12. The compound of claim 9, further comprising a non-nucleotide linker.

13-20. (canceled)

21. The compound of claim 1, wherein the antisense strand and/or sense strand of the siRNA comprise about 15 nucleotides to 25 nucleotides in length.

22-29. (canceled)

30. The compound of claim 1, wherein the siRNA comprises a double-stranded region of 15 base pairs to 20 base pairs.

31-34. (canceled)

35. The compound of claim 1, wherein the siRNA comprises a blunt-end.

36. The compound of claim 1, wherein the siRNA comprises at least one single stranded nucleotide overhang.

37-42. (canceled)

43. The compound of claim 1, wherein the siRNA comprises at least one modified internucleotide linkage.

44-51. (canceled)

52. The compound of claim 1, wherein a functional moiety is linked to the 5′ end and/or 3′ end of the siRNA antisense strand.

53-59. (canceled)

60. The compound of claim 52, wherein the functional moiety is linked to the antisense strand and/or sense strand of the siRNA by a second linker.

61-68. (canceled)

69. The compound of claim 1, wherein the antisense oligonucleotide comprises one or more modified nucleotides.

70-82. (canceled)

83. The compound of claim 1, wherein the antisense oligonucleotide comprises the formula:
A-B-C, wherein:
A comprises from about 0 to about 8 modified nucleotides, from about 2 to about 6 modified nucleotides, or 3 modified nucleotides;
B comprises from about 6 to about 18 deoxyribonucleic acid (DNA) nucleotides and/or DNA-like nucleotides, from about 6 to about 12 DNA nucleotides and/or DNA-like nucleotides, or 10 DNA nucleotides and/or DNA-like nucleotides; and
C comprises from about 0 to about 8 modified nucleotides, from about 2 to about 6 modified nucleotides, or 3 modified nucleotides;
and wherein the overall length of the antisense oligonucleotide is about 12 to about 30 nucleotides.

84-100. (canceled)

101. The compound of claim 1, comprising or consisting of:
A:
a first ASO, a first linker, a second ASO, a second linker, and a first siRNA comprising a first antisense strand and a first sense strand, wherein the 5′ end of the first ASO is linked to the 3′ end of the second ASO via the first linker and the 5′ end of the second ASO is linked to the 3′ end of the first sense strand via the second linker;
B:
a first siRNA comprising a first antisense strand and a first sense strand, a first linker, a first ASO, a second linker, and a second siRNA comprising a second antisense strand and a second sense strand, wherein the 5′ end of the first sense strand is linked to the 3′ end of the first ASO via the first linker and the 5′ end of the first ASO is linked to the 3′ end of the second sense strand via the second linker;
C:
a first ASO, a first linker, a first siRNA comprising a first antisense strand and a first sense strand, a second linker, a second ASO, a third linker, and a second siRNA comprising a second antisense strand and a second sense strand, wherein the 5′ end of the first ASO is linked to the 3′ end of the first sense strand via the first linker, the 5′ end of the first sense strand is linked to the 3′ end of the second ASO via the second linker, and the 5′ end of the second ASO is linked to the 3′ end of the second sense strand via the third linker; or
D:
a first siRNA comprising a first antisense strand and a first sense strand, a first linker, a first ASO, a second linker, a second ASO, a third linker, and a second siRNA comprising a second antisense strand and a second sense strand, wherein the 3′ end of the first sense strand is linked to the 5′ end of the first ASO via the first linker, the 3′ end of the first ASO is linked to the 3′ end of the second ASO via the second linker, and the 5′ end of the second ASO is linked to the 3′ end of the second sense strand via the third linker.

102-114. (canceled)

115. A compound comprising:
a) an siRNA comprising a sense strand and an antisense strand, and
b) a heteroduplex oligonucleotide (HDO) comprising an antisense oligonucleotide (ASO) and a complementary oligonucleotide,
wherein the siRNA is attached to the HDO with a linker.

116-149. (canceled)

150. A method for inhibiting expression of a target gene in a cell, the method comprising:
(a) introducing into the cell the compound of claim 1; and
(b) maintaining the cell produced in step (a) for a time sufficient to obtain degradation of the mRNA transcript of the target gene, thereby inhibiting expression of the target gene in the cell.

151. A method for inhibiting expression of a target gene in a subject, the method comprising administering to the subject the compound of claim 1, thereby inhibiting expression of the target gene in the subject.

152. A method for inhibiting expression of a target gene in a tissue of a subject, the method comprising administering to the subject the compound of claim 1, thereby inhibiting expression of the target gene in the tissue.

153. The method of claim 152, wherein the tissue is selected from the group consisting of lung tissue, liver tissue, placenta tissue, kidney tissue, spleen tissue, and brain tissue.

154-157. (canceled)
